(19) United States
(12) Patent Application Publication
(10) Pub. No.: US 2014/0137294 A1
(43) Pub. Date: May 15, 2014

(54) GLUCOSINOLATE TRANSPORTER PROTEIN AND USES THEREOF

(71) Applicants: Peter Denolf, Velzeke (BE); Christiane Opsomer, Nieuwpoort (BE); Barbara Ann Halkier, Copenhagen K (DK); Hussam Hassan Nour-Eldin Auis, Copenhagen K (DK); Tonni Grube Andersen, Frederiksberg (DK); Svend Roesen Madsen, Copenhagen O (DK); Morten Egevang Jørgensen, Copenhagen S (DK)

(72) Inventors: Peter Denolf, Velzeke (BE); Christiane Opsomer, Nieuwpoort (BE); Barbara Ann Halkier, Copenhagen K (DK); Hussam Hassan Nour-Eldin Auis, Copenhagen K (DK); Tonni Grube Andersen, Frederiksberg (DK); Svend Roesen Madsen, Copenhagen O (DK); Morten Egevang Jørgensen, Copenhagen S (DK)

(73) Assignees: UNIVERSITY OF COPENHAGEN, Copenhagen K. (DK); BAYER CROPSCIENCE NV, Diegem (BE)

(21) Appl. No.: 13/808,024

(22) PCT Filed: Jul. 6, 2011
(86) PCT No.: PCT/EP2011/004565

§ 371 Date: Jan. 2, 2013

Related U.S. Application Data
(60) Provisional application No. 61/362,390, filed on Jul. 8, 2010.

(30) Foreign Application Priority Data
Jul. 12, 2010 (EP) 10075299.7

(51) Int. Cl. C12N 15/82; A01H 5/10; C07K 14/415

(52) U.S. Cl. CPC C12N 15/8218; C07K 14/415; C12N 15/8251; A01H 5/10
USPC 800/285; 536/23.6; 530/379; 536/23.1; 504/241; 800/306

(57) ABSTRACT

Methods and means to alter the glucosinolate (GSL) content in plants, in particular in specific plant parts, by modifying glucosinolate transporter protein (GTR) activity in plants or parts thereof are herein described. In particular, methods are provided to decrease GSL content of plant seed and meal thereof, as well as methods to increase GSL content in green plant tissue, of Brassicales plants.

### FIELD OF THE INVENTION

The present invention relates to the field of agricultural products, especially crop plants and parts thereof having a modified glucosinolate content. Provided are methods to alter the glucosinolate (GSL) content in plants, in particular in specific plant parts, by modifying glucosinolate transporter protein (GTR) activity in plants or parts thereof. Such modification of GTR activity may be achieved through down-regulation or up-regulation of GTR gene expression or GTR protein activity. In particular, methods are provided to decrease GSL content of plant seed and meal thereof, as well as methods to increase GSL content in green plant tissue, of Brassicales plants, particularly of Brassicaceae plants such as oilseed forms of Brassica spp. (including e.g. B. napus, B. juncea and B. carinata), B. oleracea, B. rapa, cruciferous salads (including e.g. Eruca sativa and Diplotaxis tenuifolia) and Raphanus (including e.g. Raphanus sativa).

### BACKGROUND OF THE INVENTION

The Brassicales or Capparales order of plants, including the Brassicaceae or Cruciferae family, includes many cultivars that have provided mankind with a source of condiments, vegetables, forage crops, and the economically important crops rapeseed (Brassica napus and Brassica campestris or rapa) and mustard (Brassica juncea).

A striking and characteristic chemical property of these plants is their high content of glucosinolates, amino acid-derived natural plant products containing a thioglucose and a sulfonated oxime. These sulphur-containing secondary metabolites, although considered non-toxic per se, are important because of the multiplicity of physiologically active products, such as nitriles, epithionitriles, oxazolidine-2-thiones, thiocyanates and isothiocyanates, derived from them upon cleavage by the hydrolytic enzyme myrosinase (thioglucoside glucohydrolase; EC 3.2.3.1) upon plant damage (Halkier and Gershenzon, 2006, Annual Review of Plant Biology 57: 303-333).

For plants, the glucosinolate/myrosinase system protects against herbivore attacks, and is implicated in host-plant recognition by specialized predators. For humans, glucosinolates (or rather their hydrolysis products) have received increased attention as cancer-preventive agents, flavor compounds, and potential biopesticides.

Glucosinolates are present in all parts of the plant. The level of glucosinolates varies in different tissues at different developmental stages (Fieldsend and Milford, 1994, Ann. Appl. Biol. 124: 531-542; Porter et al., 1991, Ann. Appl. Biol. 118: 461-467) and is affected by external factors such as growth conditions (Zhao et al., 1993, J. Sci. Food Agric. 63: 29-37; Zhao et al., 1994, J. Sci. Food Agric. 64, 295-304), wounding (Bodnaryk, 1992, Phytochemistry 31: 2671-2677), fungal infection (Doughty et al., 1991, Ann. Appl. Biol. 118: 469-477), actual and simulated insect damage (Bodnaryk, 1992, Phytochemistry 31: 2671-2677; Koritsas et al., 1991, Ann. Appl. Biol. 118: 209-221), and other forms of stress (Mithen, 1992, Euphytica 63: 71-83). Consistent with a prominent function in plant defense, the highest glucosinolate concentrations are found in reproductive organs, including seeds, siliques, flowers and developing inflorescences, followed by young leaves, the root system and fully expanded leaves.

Glucosinolates are known to be transported in the plant from maternal tissue across several apoplastic barriers from leaves and siliques via the phloem and into embryos, where they accumulate to high levels (reviewed in Nour-Eldin and Halkier, 2009, Phytochem Rev 8: 53-67). The molecular mechanism for this transport is unknown. The transport properties of glucosinolates within plants are of interest as identification of the mechanism of transport could, for example, lead to lower levels being obtained in seeds and seed meal thereof.

Methods of plant transporter discovery are described based on homology and yeast functional complementation. However, these methods represent a limitation when identifying transporters with no known homologues or when no auxotrophic yeast strains can be engineered. Nour-Eldin (Ph. D. thesis, University of Copenhagen, Faculty of Life Sciences, Department of Plant Biology, Plant Biochemistry Laboratory, 2007, 72 p.) describes a method of plant transporter discovery based on a functional genomics approach. A normalized library of Arabidopsis secondary metabolite transporters was constructed and screened in a high-throughput manner in Xenopus oocytes. The transporter library was screened for uptake towards the aliphatic glucosinolate 4-methylthiobytyl (4-MTB) glucosinolate. Three Arabidopsis glucosinolate transporter genes were identified. Two of them (At3g47960 and At1g18880; hereinafter referred to as AtGTR1 and 3) belong to the nitrate/peptide transporter family, while the third gene (At1g71880; known as AtSUC1) belongs to the sucrose transporter family. The Arabidopsis sucrose transporter family contains 9 genes (Williams et al., 2000, Trends Plant Sci 5: 283-290), which have been shown to be broad specific and transport a variety of β-glucosides (Chandran et al., 2003, J Biol Chem 278: 44320-44325). AtSUC1, 2, 5, 8 and 9 were shown to transport 4-MTB in Xenopus oocytes at varying efficiencies (Nour-Eldin, 2007, supra). Seeds of AtSUC9 T-DNA knockout lines showed a significant reduction in four types of glucosinolates, whereas AtSUC1 knockout lines showed no difference in seed glucosinolate content (Andersen et al., 2nd Conference on Glucosinolates, May 24-27, 2009). Two additional AtGTR genes (At5g62680 and At1g69870; hereinafter referred to as AtGTR2 and AtGTR5) were identified based on homology and subsequently also shown to transport 4-MTB into Xenopus oocytes (Nour-Eldin, 2007, supra).

Among the oilseed crops currently dominating the world market, rapeseed stands out for two important reasons; its high levels of oil with excellent nutritional properties for humans, and a protein-rich seedcake meal, ideal for animal feed. Interest in reducing the levels of glucosinolates in seed results from the presence of bitter-tasting, toxic and goitrogenic degradation products which limit the incorporation of rape meal into non-ruminant animal feed (Thomson and Hughes, 1986, In Oilseed rape. Edited by Scarisbrick and Daniels. Collins, London, U.K. pp. 32-82).

Approaches to reduce glucosinolate levels in seed have thus far been through blocking biosynthetic pathways. Often, however, this approach is accompanied by adverse effects on plant fitness due to e.g. increased susceptibility to biotic or abiotic stresses.

In the 1970s, traditional breeding generated a multiple-loci-dependent B. napus cultivar with reduced glucosinolate content in all parts of the plant, including the seeds. This “00” (“double low”) variety and its descendants have subsequently become the most widely grown rapeseed cultivars across the northern hemisphere. The prolonged selection bottleneck caused by this single source has, however, created a limited genetic diversity for future B. napus breeding programs and limits interspecific hybridization. This poses a serious problem for B. napus breeders striving to improve yield and disease resistance as well as to introduce novel traits such as drought tolerance through interspecific hybridizations.

A further problem with “00” varieties is that the seeds, although low in glucosinolates, are not free of them. Pressed seed cake obtained from “00” varieties after the oil has been extracted will typically contain less than 18-24 micromoles of total glucosinolates (GSL) per gram of dry weight (as compared to traditional rapeseed meal that contains 120-150 pmol of total GSL per gram). For use in compound feed, palatability to ruminants sets the level of total GSL permitted at no more than 10-15 micromoles per gram of dry weight, meaning an animal feed could in theory be compounded almost entirely of “00” pressed seed cake if the seed cake is at the lower level of GSL content. However, it has recently been found that poultry and pigs are both much more sensitive to levels of GSLs than ruminants, and more than 2-4 micromoles GSL per gram of dry weight in the feed can severely affect reproductive efficiencies in these animals. A truly “zero GSL” variety would be of significant commercial advantage to animal feed compounders, producers of pressed seed cake and the growers by increasing quantities of seed cake that can be included in compound feeds and removing the need for continual monitoring of GSL levels in their products.

These and other problems are solved as hereinafter described in the different embodiments, examples and claims.

### SUMMARY OF THE INVENTION

The present invention relates to plants and plant parts, such as seed, seed meal, green plant tissue and root tissues, with modified total glucosinolate (GSL) content. Provided are methods to produce plants and plant parts with modified total GSL content by modification of functional glucosinolate transport protein (GTR) activity. In particular, methods are provided to produce seed with decreased total GSL content and methods to produce green plant tissue, such as leaf tissue, with increased total GSL content by reduction of functional GTR activity. Further provided is the use of GTR-encoding nucleic acid molecules to obtain modified GSL content in plants and plant parts, and the use of plants and plant parts with modified functional GTR activity, for example, in cancer-prevention, in pest management or in animal feeding.

In a first aspect of the invention reduction of functional GTR activity may be achieved through down-regulation of GTR gene expression. In one embodiment of the invention, a method is provided to modify total GSL content of plants and plant parts by introduction of an RNA molecule capable of down-regulating GTR gene expression, e.g. through introduction of a chimeric nucleic acid construct comprising a nucleotide region which upon expression yields such RNA molecule.

In one embodiment, GTR gene expression is down-regulated by introducing an RNA molecule comprising part of a GTR-encoding nucleotide sequence or a homologous sequence or by introducing a chimeric DNA encoding such RNA molecule. In another embodiment, GTR gene expression is down-regulated by introducing an antisense RNA molecule comprising a nucleotide sequence complementary to at least part of a GTR-encoding nucleotide or homologous sequence, or by introducing a chimeric DNA encoding such RNA molecule. In yet another embodiment, GTR gene expression is down-regulated by introducing a double-stranded RNA molecule comprising a sense and an antisense RNA region corresponding to and respectively complementary to at least part of a GTR gene sequence, which sense and antisense RNA region are capable of forming a double stranded RNA region with each other. In another embodiment, GTR gene expression can be down-regulated by introduction of a microRNA molecule (which may be processed from a pre-microRNA molecule) capable of guiding the cleavage of GTR mRNA. Again, microRNA molecules may be conveniently introduced into plant cells through expression from a chimeric DNA molecule encoding such miRNA, pre-miRNA or primary miRNA transcript.

In another embodiment of the invention, a method is provided to modify total GSL content of plants or plant parts by down-regulation of GTR gene expression through alteration of the nucleotide sequence of the endogenous GTR gene, such as e.g. alterations in regulatory signals including promoter sequence, intron processing signals, untranslated leader and trailer sequence or polyadenylation signal sequences.

In a second aspect of the invention, reduction of functional GTR activity may occur at the level of the GTR activity. In one embodiment of the invention, a method is provided to modify total GSL content of plants and plant parts by introduction of a chimeric nucleic acid construct encoding a protein capable of down-regulating GTR protein activity. In one embodiment, GTR protein activity may be down-regulated by expression of a dominant negative GTR gene. In another embodiment of the invention, GTR protein activity may be down-regulated by expression of a GTR-inactivating antibody. Functional GTR activity may also be modulated by changing the phosphorylation/deposphorylation status of GTR proteins. This can be achieved by using variant alleles of GTR encoding genes, whereby the phosphorylation sites are modified, as hereinafter described.

In another embodiment of the invention, a method is provided to modify total GSL content of plants or plant parts by down-regulation of GTR protein activity through alteration of the nucleotide sequence of the endogenous GTR gene e.g. through alterations in the coding region introducing insertions, deletions or substitutions of amino acids, truncations of the encoded protein or splice site mutations.

### BRIEF DESCRIPTION OF THE FIGURES

FIG. 1a: Unrooted phylogenetic tree of protein sequences in the Arabidopsis NRT1/PTR family. Protein sequences were retrieved from the ARAMEMNON plant membrane protein database (Schwacke et al., Plant Physiol. 131: 16-26). The At3g47960 (AtGTR1) protein subclade is marked with a bracket. Grey shading of circles reflects relative in vitro GSL uptake activity in Xenopus oocytes with white indicating zero uptake and black maximum uptake observed. Unmarked genes have not been tested. The phylogenetic relationship was inferred using the Neighbor-Joining method (Saitou and Nei, 1987, Mol Biol Evol. 4(4):406-25). The phylogenetic relationship was computed using the Poisson correction method (Zuckerkandl and Pauling, 1965, In Bryson V, Vogel HJ (eds), Academic Press, New York, pp 97-166). All positions containing gaps and missing data were eliminated from the dataset (Complete deletion option). There were a total of 300 positions in the final dataset. Phylogenetic analyses were conducted in MEGA4 (Tamura et al., 2007, Mol Biol Evol 24: 1596-1599).

FIG. 1b: Alignment of Arabidopsis GTR amino acid sequences. POT1-6_AT..p: AtGTR1-6 (SEQ ID Nos 2, 4, 6, 8, 10 and 12)

FIG. 1c: Alignment of Arabidopsis GTR amino acid sequences 1 to 6 including the N-terminal extension of 30 amino acids in GTR1 (SEQ ID Nos 142, 4, 6, 8, 10 and 12).

FIG. 2: 4-MTB uptake in Xenopus oocytes expressing BrGTR2-A2 proteins with substitutions of amino acids corresponding to the amino acid at position 126 (Gly to Arg), 145 (Gly to Arg), 192 (Glu to Lys), 229 (Trp to STOP) and 359 (Ser to Phe) of SEQ ID NO: 66 and wildtype BrGTR2-A2 protein. Assay conditions: 0.5 mM 4-MTB for one hour. Each bar is the mean of 4 replicates, error is standard deviation.

FIG. 3: Biochemical analysis of AtGTR1 and 2 mediated GSL transport in Xenopus oocytes. A and B, pH dependence of AtGTR-mediated GSL transport. A, AtGTR2 mediated 4-MTB uptake measured by LC-MS analysis of oocyte extracts, n=3 oocytes. B, AtGTR1 currents induced by 1 mM 4-MTB were measured at different membrane potentials at pH 5 (), pH 6 (♦), and pH 7 (▴). Currents were normalized to the current induced at pH 5 at −60 mV, error bars are SD, n=4 oocytes. C and D, kinetic analysis of AtGTR-mediated glucosinolate transport. Normalized glucosinolate dependent currents at a membrane potential of −60 mV are plotted against 4-MTB concentrations, line indicates a fit of the Michaelis-Menten equation to the data; error bars are SD. For AtGTR1 (n=6 oocytes); For AtGTR2 (n=5 oocytes). Each oocyte-dataset were normalized to current elicited at 100 μM 4-MTB. E and F, substrate specificity of AtGTRs. E, AtGTR1 and AtGTR2 uptake of 0.04 mM C-14 labeled pOHBG at pH 5 in the presence of 2 mM 4-MTB, 2 mM NO₃⁻, 2 mM glucose+2 mM SO₄⁻⁻, or a solution of 2 mM Ala-His+2 mM Asp-Ala+2 mM Gly-Leu+2 mM Ala-Phe+2 mM Gly-Glu+2 mM Gly-Gly-Gly, error bars are SD, n=8 oocytes. F, AtGTR1 currents induced by endogenous and exogenous glucosinolates, representative positive, negative and neutral dipeptides and two tripeptides Currents were normalized to the current induced by 100 μM 4-MTB at pH 5 clamped at −60 mV, error bars are SD, n=4 oocytes.

FIG. 4: Glucosinolate content in seeds from Arabidopsis wild type and GTR knockout plants. A-D, HPLC trace showing GSL content in total seeds from a representative silique from wildtype, atgtr1, atgtr2 and atgtr1/atgtr2 plants, respectively. A, 54 seeds from wildtype plants. B, 51 seeds from atgtr2. C, 53 seeds from atgtr1. D, 39 seeds from atgtr1/atgtr2 plants. E, 20 seeds from atgtr1/atgtr2 plant transformed with native AtGTR2 complementation construct. Abbreviations denote the prefix of detected glucosinolates. 4ohb: 4-hydroxybutyl-GSL, 4 msb: 4-methylsulfinylbutyl-GSL, 5msp: 5-methyldsulfinylpentyl-GSL, 6msh: 6 methylsulfinylhexyl-GSL, 7msh: 7-methylsulfinylheptyl-GSL, 4mtb: 4-methylthiobutyl-GSL, 8mso: 8-methylsulfinyl-GSL, i3m: indol-3-yl-methyl-GSL, 5mtp: 5-methylthiobutyl-GSL, 3bzop: 3-benzoylpropyl-GSL, 4bzob: 4-benzoylbutyl-GSL, 7mth: 7-methylthioheptyl-GSL, 8-methylthiooctyl-GSL. 2-propenyl-GSL: internal standard GSL, normalizing for loss during purification.

FIG. 5: Total aliphatic glucosinolate (abbreviated as “gls” on the Y-axis) content in different plant parts from soil grown Arabidopsis plants through development. Harvesting time points: before bolting: 3 week old plants, after bolting (and onset of silique development): 5 week old plants, senescence (and seed maturity): 8 week. For time points “after bolting” and “senescence” tissues are harvested from the same plants. A, seeds. B, rosette. C, Stems (including flowers), total cauline leaves, total intact siliques (including developing and mature seeds). D, Single silique walls. Bars represent mean±SD. (Total aliphatic GSLs include all methionine-derived GSLs including benzoyloxy-GSLs).

FIG. 6: Total aliphatic glucosinolate (abbreviated as “gls” on the Y-axis) content in different plant parts from hydroponically grown Arabidopsis plants through development. Harvesting time points as stated for soil grown plants in FIG. 2. A, roots. B, rosette bolting. C, aliphatic glucosinolate concentration per mg plant.

FIG. 7: Detail of alignment of part of Arabidopsis GTR amino acid sequences: AtGTR2: SEQ ID NO: 4 from amino acid position 454 to amino acid position 493; BrGTR2: SEQ ID NO: 26 from amino acid position 450 to amino acid position 489; AtGTR1: SEQ ID NO: 2 from amino acid position 440 to amino acid position 479 (or SEQ ID No 142 from amino acid position 470 to 509); AtGTR3: SEQ ID NO: 6 from amino acid position 438 to amino acid position 467; AtGTR5: SEQ ID NO: 10 from amino acid position 463 to amino acid position 495; AtGTR6: SEQ ID NO: 12 from amino acid position 423 to amino acid position 449; and AtGTR4: SEQ ID NO: 8 from amino acid position 425 to amino acid position 451.

FIG. 8: Predicted protein structure for GTR2 protein with relative indication of the mutations in the mutant BrnTR2 protein. The dashed arrow indicates a loop characteristic for GTR1, GTR2 and GTR3 proteins (also indicated by the boxed sequences in FIG. 7.

FIG. 9: 3-butenyl content in B. rapa seeds. The X-axis shows the BrGTR2-genotype of seeds; darker columns indicate seeds from homozygous mutants, whereas lighter columns indicate seeds from wild type segregants (WTS) for the respective mutations. WT represent unmutated reference wild type seeds. The Y-axis shows 3-butenyl concentration in nmol/mg. Panel A) Glucosinolate concentration of seeds from each plant; standard deviations are calculated from n=3 seeds. Panel B) Average glucosinolate concentration of all “W229X-” (stop codon) mutants, with standard deviations calculated from n=27 seeds, n=12 seeds and n=3seeds, respectively.

FIG. 10: Total glucosinolate content in mutant and reference B. napus seeds. The Y axis shows the genotype of the seeds. Reference are unmutated isogenic wild-type seeds.

FIG. 11: 4-MTB uptake of phosphorylation/dephosphorylation mimicking constructs in comparison with wild type At GTR1 and GTR2; X-axis represents the genotypes of the different constructs.

FIG. 12: Concentration of glucosinolates in various tissues in Brassica rapa. The X-axis shows plant part analyzed and the Y-axis shows nmol glucosinolate/mg freshweight. The analysis shows that glucosinolate concentration in entire Brgtr2 plants is higher than in WTs and that this increased concentration are caused by the glucosinolate levels in leaves and siliques, not stem and root. Standard deviations are calculated from n=5.

FIG. 13: Freshweight of various tissues. The X-axis shows plant part weighed and the Y-axis shows mg freshweight. The analysis shows that Brgtr2 plants are larger than the WTs, and that this increase is primarily caused by the weights of leaves and stem. It should be noted that the low weight of BrGTR2WT siliques is partly caused by a low amount of seeds in these compared to true WT and Brgtr2 siliques (data not shown). Standard deviations are calculated from n=5.

### GENERAL DEFINITIONS

“Glucosinolates” (abbreviated herein as “GSLs” or “GLSs”), as used herein, refers to amino acid-derived thioglucosidic organic anions comprising a sulfonated aldoxime moiety. A variable side chain depending on the parent amino acid and further side chain modifications gives the distinct chemical and biological properties for GSLs. Approximately 120 different GSLs have been described in the literature and they are all derived from only 8 different amino acids. The parent amino acids are conveniently used as a classification criteria. GSLs derived from Ala, Leu, Ile, Val and Met are called “aliphatic GSLs”, those derived from Tyr and Phe are called “aromatic GSLs” and those derived from Trp are called “indole GSLs”. The great variety in GSL types is caused by a number of modifications on the side chain of the parent amino acid. Especially, methionine undergoes a wide range of transformations. The predominant aliphatic GSLs in the Brassicaceae possess side chains derived from chain elongated forms of Met, such as aliphatic thio-GSLs 3-methylthiopropyl (3-MTP)-, 4-methylthiobutyl (4-MTB)-, 5-methylthiopentyl (5-MTP)-, 6-methylthiohexyl (6-MTH)-, 7-methylthioheptyl (7-MTH)- and 8-methylthiooctyl (8-MTO)-GSL; aliphatic sulfinyl-GSLs 3-methylsulfinylpropyl (3-MSP)-, 4-methylsulfinylbutyl (4-MSB)-, 5-methylsulfinylpentyl (5-MSP)-, 6-methylsulfinylhexyl (6-MSH)-, 7-methylsulfinylheptyl (7-MSH)- and 8-methylsulfinyloctyl (8-MSO)-GSL; aliphatic hydroxy-GSLs 3-hydroxypropyl (3-OHP)- and 4-hydroxybutyl (4-OHB)-GSL; aliphatic benzoyloxy-GSLs 3-benzoyloxypropyl (3-BZOP)- and 4-benzoyloxybutyl (4-BZOB)-GSL, and aliphatic alkenyl-GSLs 2-propenyl (2-P)- and 3-butenyl (3-B)-GSL. The predominant aromatic GSLs in the Brassicaceae possess side chains derived from Phe, such as aromatic GSL 2-phenylethyl (2-PE)-GSL. Lower amounts of GSLs with indolylic side chains derived from Trp, such as indol-GSL indol-3-ylmethyl (i3M)-GSL, also occur. GSLs co-occur in plants with the GSL-specific thioglucosidase myrosinase. This enzyme is physically separated from GSLs in plants, but is brought into contact with its substrate upon tissue disruption. The resulting hydrolysis product consists of one free glucose and one aglycone molecule per GSL molecule. The agclycones are unstable and readily rearrange into isothiocyanates, nitriles, thiocyanates and other more or less toxic compounds. Depending on the side chain of the parent amino acid these hydrolysis products contribute the actual biological activity of GSLs, while intact GSLs are believed to be an inactive storage form.

As used herein, “glucosinolate content” of a plant or plant part refers to the total of GSLs, including aliphatic, aromatic and indole GSLs, without regard to the type of GSLs. Thus the “total GSL content” or “GSL content” of a plant or plant part means the content of total GSLs of that plant or plant part and is expressed on a molecular (nmol/g or pmol/g) basis (rather than on a weight (mg/kg) basis) as GSLs have significantly different molecular weights depending on the size of their side chain. GSL accumulation varies between tissues and developmental stages. Young leaves and reproductive tissues such as siliques and seeds contain the highest concentrations while senescing leaves contain the lowest concentrations of GSLs. Intermediate concentrations are found throughout the “large” organs such as the roots, leaves and stem. In addition, the composition of the GSL profile varies markedly between organs. In roots and vegetative tissues, the GSL content is composed of indole and aliphatic GSLs while the aromatic are absent. In siliques and seeds, small amounts of aromatic and indole GSLs are found while the rest of the GSL content is entirely composed of aliphatic GSLs.

“Canola”, “double-zero rapeseed” or “double-low rapeseed” is an offspring of rapeseed (Brassica napus and Brassica campestris or rapa) which was bred through standard plant breeding techniques to have low levels of erucic acid (below 2%) in the oil portion and low levels of GSLs (below 30 pmol/g) in the meal portion. “Seed” of (double-zero) rapeseed is small and round, 1-2 mm in diameter. It contains approximately 42-43% oil, which is extracted for use as edible vegetable oil. The remaining “seed meal” is a widely used protein source in animal feeds. The GSLs in rapeseed were reduced because they are toxic and unpalatable to most animals, and therefore limit the inclusion level of rapeseed meal in animal feeds to very low levels. Canola and rapeseed meals are commonly used in animal feeds around the world and are sold in bulk form as a mash or in pellets.

A “decrease in total GSL content” or “increase in total GSL content” of a plant or plant part by the methods of the present invention is measured relative to the total GSL content of a reference plant or plant part with similar genetic background. Total GSL content can be measured by any appropriate method. Methods to quantify total GSL content and to determine GSL composition of plant material are well known in the art and include but are not limited to: HPLC-UV desulfo-method involving HPLC analysis of methanol extracts desulfated and eluted from sephadex anion exchange columns as described by, e.g., Hansen et al. (2007, Plant J. 50 (5): 902-910); analysis of intact GSLs by MALDI-TOF mass spectrometry as described by, e.g., Botting et al. (2002, J. Agric. Food Chem. 50 (5): 983-988); near-infrared reflectance spectroscopy as described by, e.g., Font et al. (2005, J. Agric. Sci. 143: 65-73); methods yielding spectrophotometrically active degradation products as summarized by, e.g., Clarke (2010, Anal. Methods 2: 310-325); HPLC mass spectrometry analysis of intact glucosinolates as described by, e.g., Rochfort et al. (2008, Phytochemistry 69: 1671).

“Biofumigation”, as used herein, refers to the use of GSL-containing plants, such as Brassicaceae (e.g. cabbage, cauliflower, kale and mustard), Capparidaceae (e.g. cleome) and Moringaceae (e.g. horse-radish) species, as biologically-active rotation and green manure crop for controlling several soil-borne pathogens and diseases.

“Crop plant” refers to plant species cultivated as a crop, such as Brassica napus (AACC, 2n=38), Brassica juncea (AABB, 2n=36), Brassica carinata (BBCC, 2n=34), Brassica rapa (syn. B. campestris) (AA, 2n=20), Brassica oleracea (CC, 2n=18) or Brassica nigra (BB, 2n=16). The definition does not encompass weeds, such as Arabidopsis thaliana.

The term “nucleic acid” or “nucleic acid molecule” refers to a DNA or RNA molecule in single or double stranded form, particularly a DNA encoding a protein or protein fragment according to the invention. An “endogenous nucleic acid” refers to a nucleic acid within a plant cell, e.g. an endogenous allele of a GTR gene present within the nuclear genome of a Brassica cell. An “isolated nucleic acid” is used to refer to a nucleic acid that is no longer in its natural environment, for example in vitro or in a recombinant bacterial or plant host cell.

The term “gene” means a DNA fragment comprising a DNA region (transcribed region), which is transcribed into an RNA molecule (e.g. into a pre-mRNA, comprising intron sequences, which is then spliced into a mature mRNA, or directly into a mRNA without intron sequences, or into a pre-miRNA) in a cell, operable linked to regulatory regions (e.g. a promoter). A gene may thus comprise several operably linked DNA fragments, such as a promoter, a 5′ leader sequence comprising e.g. sequences involved in translation initiation, a (protein) coding region (cDNA or genomic DNA) and a 3′ non-translated sequence comprising e.g. transcription termination sites. “Endogenous gene” is used to differentiate from a “foreign gene”, “transgene” or “chimeric gene”, and refers to a gene from a plant of a certain plant genus, species or variety, which has not been introduced into that plant by transformation (i.e. it is not a “transgene”), but which is normally present in plants of that genus, species or variety, or which is introduced in that plant from plants of another plant genus, species or variety, in which it is normally present, by normal breeding techniques or by somatic hybridization, e.g., by protoplast fusion. Similarly, an “endogenous allele” of a gene is not introduced into a plant or plant tissue by plant transformation, but is, for example, generated by plant mutagenesis and/or selection or obtained by screening natural populations of plants.

As used herein a “chimeric nucleic acid construct” refers to a nucleic acid construct which is not normally found in a plant species. A chimeric nucleic acid construct can be DNA or RNA. “Chimeric DNA construct” and “chimeric gene” are used interchangeably to denote a gene which is not normally found in a plant species or to refer to any gene in which the promoter or one or more other regulatory regions of the gene are not associated in nature with part or all of the transcribed DNA region.

“Expression of a gene” or “gene expression” refers to the process wherein a DNA region, which is operably linked to appropriate regulatory regions, particularly a promoter, is transcribed into an RNA molecule. The RNA molecule is then processed further (by post-transcriptional processes) within the cell, e.g. by RNA splicing and translation initiation and translation into an amino acid chain (polypeptide), and translation termination by translation stop codons. The term “functionally expressed” is used herein to indicate that a functional protein is produced; the term “not functionally expressed” to indicate that a protein with significantly reduced or no functionality (biological activity) is produced or that no protein is produced (see further below). An RNA molecule is biologically active when it is either capable of interaction with another nucleic acid or protein or which is capable of being translated into a biologically active polypeptide or protein. A gene is said to encode an RNA when the end product of the expression of the gene is biologically active RNA, such as e.g. an antisense RNA, a ribozyme, or a miRNA. A gene is said to encode a protein when the end product of the expression of the gene is a protein or polypeptide. A gene is said to encode a GTR-inhibitory RNA when the end product of the expression of the gene is capable of down-regulating GTR functional activity, i.e. capable of down-regulating GTR gene expression and/or GTR protein activity.

For the purpose of the invention, the term “plant-operative promoter” and “plant-expressible promoter” mean a promoter which is capable of driving transcription in a plant, plant tissue, plant organ, plant part, or plant cell. This includes any promoter of plant origin, but also any promoter of non-plant origin which is capable of directing transcription in a plant cell.

Promoters that may be used in this respect are constitutive promoters, such as the promoter of the cauliflower mosaic virus (CaMV) 35S transcript (Harpster et al., 1988, Mol. Gen. Genet. 212: 182-190), the CaMV 19S promoter (U.S. Pat. No. 5,352,605; WO 84/02913; Benfey et al., 1989, EMBO J. 8:2195-2202), the subterranean clover virus promoter No 4 or No 7 (WO 96/06932), the Rubisco small subunit promoter (U.S. Pat. No. 4,962,028), the ubiquitin promoter (Holtorf et al., 1995, Plant Mol. Biol. 29:637-649), T-DNA gene promoters such as the octopine synthase (OCS) and nopaline synthase (NOS) promoters from Agrobacterium, and further promoters of genes whose constitutive expression in plants is known to the person skilled in the art.

Further promoters that may be used in this respect are tissue-specific or organ-specific promoters, preferably seed-specific promoters, such as the 2S albumin promoter (Joseffson et al., 1987, J. Biol. Chem. 262:12196-12201), the phaseolin promoter (U.S. Pat. No. 5,504,200; Bustos et al., 1989, Plant Cell 1.(9):839-53), the legumine promoter (Shirsat et al., 1989, Mol. Gen. Genet. 215(2):326-331), the “unknown seed protein” (USP) promoter (Baumlein et al., 1991, Mol. Gen. Genet. 225(3):459-67), the napin promoter (U.S. Pat. No. 5,608,152; Stalberg et al., 1996, Planta 199:515-519), the Arabidopsis oleosin promoter (WO 98/45461), the Brassica Bce4 promoter (WO 91/13980), and further promoters of genes whose seed-specific expression in plants is known to the person skilled in the art.

Other promoters that can be used are tissue-specific or organ-specific promoters like organ primordia-specific promoters (An et al., 1996, Plant Cell 8: 15-30), stem-specific promoters (Keller et al., 1988, EMBO J. 7(12): 3625-3633), leaf-specific promoters (Hudspeth et al., 1989, Plant Mol. Biol. 12: 579-589), mesophyl-specific promoters (such as the light-inducible Rubisco promoters), root-specific promoters (Keller et al., 1989, Genes Dev. 3: 1639-1646), tuber-specific promoters (Keil et al., 1989, EMBO J. 8(5): 1323-1330), vascular tissue-specific promoters (Peleman et al., 1989, Gene 84: 359-369), stamen-selective promoters (WO 89/10396, WO 92/13956), dehiscence zone-specific promoters (WO 97/13865), and the like.

Chimeric RNA constructs according to the invention may be delivered to plant cells using means and methods such as described in WO90/12107, WO03/052108 or WO2005/098004.

The terms “protein” or “polypeptide” are used interchangeably and refer to molecules consisting of a chain of amino acids, without reference to a specific mode of action, size, 3-dimensional structure or origin. A “fragment” or “portion” of a GTR protein may thus still be referred to as a “protein”. An “isolated protein” is used to refer to a protein that is no longer in its natural environment, for example in vitro or in a recombinant bacterial or plant host cell.

The term “transporter protein” is used to refer to a transmembrane protein that helps a certain substance or class of closely related substances to cross the membrane. A “glucosinolate transporter protein” (abbreviated herein as “GTR”) is a proton-dependent oligopeptide transporter (POT) protein involved in glucosinolate transport.

The term “GTR gene” refers herein to a nucleic acid sequence encoding a glucosinolate transporter (GTR) protein.

As used herein, the term “allele(s)” means any of one or more alternative forms of a gene at a particular locus. In a diploid (or amphidiploid) cell of an organism, alleles of a given gene are located at a specific location or locus (loci plural) on a chromosome. One allele is present on each chromosome of the pair of homologous chromosomes.

As used herein, the term “homologous chromosomes” means chromosomes that contain information for the same biological features and contain the same genes at the same loci but possibly different alleles of those genes. Homologous chromosomes are chromosomes that pair during meiosis. “Non-homologous chromosomes”, representing all the biological features of an organism, form a set, and the number of sets in a cell is called ploidy. Diploid organisms contain two sets of non-homologous chromosomes, wherein each homologous chromosome is inherited from a different parent. In amphidiploid species, essentially two sets of diploid genomes exist, whereby the chromosomes of the two genomes are referred to as “homeologous chromosomes” (and similarly, the loci or genes of the two genomes are referred to as homeologous loci or genes). A diploid, or amphidiploid, plant species may comprise a large number of different alleles at a particular locus.

As used herein, the term “heterozygous” means a genetic condition existing when two different alleles reside at a specific locus, but are positioned individually on corresponding pairs of homologous chromosomes in the cell. Conversely, as used herein, the term “homozygous” means a genetic condition existing when two identical alleles reside at a specific locus, but are positioned individually on corresponding pairs of homologous chromosomes in the cell.

As used herein, the term “locus” (loci plural) means a specific place or places or a site on a chromosome where for example a gene or genetic marker is found. For example, the “GTR-A1 locus” refers to the position on a chromosome of the A genome where the GTR-A1 gene (and two GTR-A1 alleles) may be found, while the“GTR-C1 locus” refers to the position on a chromosome of the C genome where the GTR-C1 gene (and two GTR-C1 alleles) may be found.

Whenever reference to a “plant” or “plants” according to the invention is made, it is understood that also plant parts, progeny of the plants which retain the distinguishing characteristics of the parents (especially the glucosinolate content in particular plant parts), such as seed obtained by selfing or crossing, e.g. hybrid seed (obtained by crossing two inbred parental lines), hybrid plants and plant parts derived thereof are encompassed herein, unless otherwise indicated.

“Plant parts”, as used herein, refers to any part of the plant, including plant cells, plant tissues, plant organs, siliques or seed pods, seeds, severed parts such as roots, leaves, flowers, pollen, etc.).

A “molecular assay” (or test) refers herein to an assay that indicates (directly or indirectly) the presence or absence of one or more particular GTR alleles at one or both GTR loci (e.g. at one or both of the GTR-A1 or GTR-C1 loci). In one embodiment it allows one to determine whether a particular (wild type or mutant) allele is homozygous or heterozygous at the locus in any individual plant.

“Wild type” (also written “wildtype” or “wild-type”), as used herein, refers to a typical form of a plant or a gene as it most commonly occurs in nature. A “wild type plant” refers to a plant with the most common phenotype of such plant in the natural population. A “wild type allele” refers to an allele of a gene required to produce the wild-type phenotype. By contrast, a “mutant plant” refers to a plant with a different rare phenotype of such plant in the natural population or produced by human intervention, e.g. by mutagenesis, and a “mutant allele” refers to an allele of a gene required to produce the mutant phenotype.

As used herein, the term “wild type GTR” (e.g. wild type GTR-A1 or GTR-C1), means a naturally occurring GTR allele found within plants, in particular Brassicaceae plants, especially Arabidopsis and Brassica plants, which encodes a functional GTR protein (e.g. a functional GTR-A1 or GTR-C1, respectively). In contrast, the term “mutant GTR” (e.g. mutant GTR-A1 or GTR-C1), as used herein, refers to an GTR allele, which does not encode a functional GTR protein, i.e. an GTR allele encoding a non-functional GTR protein (e.g. a non-functional GTR-A1 or GTR-C1, respectively), which, as used herein, refers to an GTR protein having no biological activity or a significantly reduced biological activity as compared to the corresponding wild-type functional GTR protein, or encoding no GTR protein at all. Such a “mutant GTR allele” (also called “full knock-out” or “null” allele) is a wild-type GTR allele, which comprises one or more mutations in its nucleic acid sequence, whereby the mutation(s) preferably result in a significantly reduced (absolute or relative) amount of functional GTR protein in the cell in vivo. Mutant alleles of the GTR protein-encoding nucleic acid sequences are designated as “gtr” (e.g. gtr-a1 or gtr-c1, respectively) herein. Mutant alleles can be either “natural mutant” alleles, which are mutant alleles found in nature (e.g. produced spontaneously without human application of mutagens) or “induced mutant” alleles, which are induced by human intervention, e.g. by mutagenesis.

A “significantly reduced amount of functional GTR protein” (e.g. functional GTR-A1 or GTR-C1 protein) refers to a reduction in the amount of a functional GTR protein produced by the cell comprising a mutant GTR allele by at least 30%, 40%, 50%, 60%, 70%, 80%, 90%, 95% or 100% (i.e. no functional GTR protein is produced by the cell) as compared to the amount of the functional GTR protein produced by the cell not comprising the mutant GTR allele. This definition encompasses the production of a “non-functional” GTR protein (e.g. truncated GTR protein) having no GSL transport activity in vivo, the reduction in the absolute amount of the functional GTR protein (e.g. no functional GTR protein being made due to the mutation in the GTR gene), and/or the production of an GTR protein with significantly reduced GSL transport activity compared to the activity of a functional wild type GTR protein (such as an GTR protein in which one or more amino acid residues that are crucial for the GSL transport activity of the encoded GTR protein, as exemplified below, are substituted for another amino acid residue). The term “mutant GTR protein”, as used herein, refers to an GTR protein encoded by a mutant GTR nucleic acid sequence (“gtr allele”) whereby the mutation results in a significantly reduced and/or no GTR activity in vivo, compared to the activity of the GTR protein encoded by a non-mutant, wild type GTR sequence (“GTR allele”).

“Mutagenesis”, as used herein, refers to the process in which plant cells (e.g., a plurality of Brassica seeds or other parts, such as pollen, etc.) are subjected to a technique which induces mutations in the DNA of the cells, such as contact with a mutagenic agent, such as a chemical substance (such as ethylmethylsulfonate (EMS), ethylnitrosourea (ENU), etc.) or ionizing radiation (neutrons (such as in fast neutron mutagenesis, etc.), alpha rays, gamma rays (such as that supplied by a Cobalt 60 source), X-rays, UV-radiation, etc.), or a combination of two or more of these. Thus, the desired mutagenesis of one or more GTR alleles may be accomplished by use of chemical means such as by contact of one or more plant tissues with ethylmethylsulfonate (EMS), ethylnitrosourea, etc., by the use of physical means such as x-ray, etc, or by gamma radiation, such as that supplied by a Cobalt 60 source. While mutations created by irradiation are often large deletions or other gross lesions such as translocations or complex rearrangements, mutations created by chemical mutagens are often more discrete lesions such as point mutations. For example, EMS alkylates guanine bases, which results in base mispairing: an alkylated guanine will pair with a thymine base, resulting primarily in G/C to A/T transitions.

As used herein, the term “non-naturally occurring” when used in reference to a plant, means a plant with a genome that has been modified by man. A transgenic plant, for example, is a non-naturally occurring plant that contains an exogenous nucleic acid molecule, e.g., a chimeric gene comprising a transcribed region which when transcribed yields a biologically active RNA molecule capable of reducing the expression of an endogenous gene, such as an GTR gene according to the invention, and, therefore, has been genetically modified by man. In addition, a plant that contains a mutation in an endogenous gene, for example, a mutation in an endogenous GTR gene, (e.g. in a regulatory element or in the coding sequence) as a result of an exposure to a mutagenic agent is also considered a non-naturally plant, since it has been genetically modified by man. Furthermore, a plant of a particular species, such as Brassica that contains mutation in an endogenous gene, for example, in an endogenous GTR gene, that in nature does not occur in that particular plant species, as a result of, for example, directed breeding processes, such as marker-assisted breeding and selection or introgression, with a plant of the same or another species, such as Brassica juncea or rapa, of that plant is also considered a non-naturally occurring plant. In contrast, a plant containing only spontaneous or naturally occurring mutations, i.e. a plant that has not been genetically modified by man, is not a “non-naturally occurring plant” as defined herein and, therefore, is not encompassed within the invention. One skilled in the art understands that, while a non-naturally occurring plant typically has a nucleotide sequence that is altered as compared to a naturally occurring plant, a non-naturally occurring plant also can be genetically modified by man without altering its nucleotide sequence, for example, by modifying its methylation pattern.

The term “ortholog” of a gene or protein refers herein to the homologous gene or protein found in another species, which has the same function as the gene or protein, but is (usually) diverged in sequence from the time point on when the species harboring the genes diverged (i.e. the genes evolved from a common ancestor by speciation). Orthologs of, for example, the Brassica napus GTR genes may thus be identified in other plant species (e.g. Brassica juncea, etc.) based on both sequence comparisons (e.g. based on percentages sequence identity over the entire sequence or over specific domains) and/or functional analysis.

A “variety” is used herein in conformity with the UPOV convention and refers to a plant grouping within a single botanical taxon of the lowest known rank, which grouping can be defined by the expression of the characteristics resulting from a given genotype or combination of genotypes, can be distinguished from any other plant grouping by the expression of at least one of the said characteristics and is considered as a unit with regard to its suitability for being propagated unchanged (stable).

The term “comprising” is to be interpreted as specifying the presence of the stated parts, steps or components, but does not exclude the presence of one or more additional parts, steps or components. A plant comprising a certain trait may thus comprise additional traits. A nucleic acid or protein comprising a sequence of nucleotides or amino acids, may comprise more nucleotides or amino acids than the actually cited ones, i.e., be embedded in a larger nucleic acid or protein. A chimeric gene comprising a DNA region which is functionally or structurally defined may comprise additional DNA regions etc.

It is understood that when referring to a word in the singular (e.g. plant or root), the plural is also included herein (e.g. a plurality of plants, a plurality of roots). Thus, reference to an element by the indefinite article “a” or “an” does not exclude the possibility that more than one of the element is present, unless the context clearly requires that there be one and only one of the elements. The indefinite article “a” or “an” thus usually means “at least one”.

For the purpose of this invention, the “sequence identity” of two related nucleotide or amino acid sequences, expressed as a percentage, refers to the number of positions in the two optimally aligned sequences which have identical residues (×100) divided by the number of positions compared. A gap, i.e., a position in an alignment where a residue is present in one sequence but not in the other, is regarded as a position with non-identical residues. The “optimal alignment” of two sequences is found by aligning the two sequences over the entire length according to the Needleman and Wunsch global alignment algorithm (Needleman and Wunsch, 1970, J Mol Biol 48(3):443-53) in The European Molecular Biology Open Software Suite (EMBOSS, Rice et al., 2000, Trends in Genetics 16(6): 276-277; see e.g. http://www.ebi.ac.uk/emboss/align/index.html) using default settings (gap opening penalty=10 (for nucleotides)/10 (for proteins) and gap extension penalty=0.5 (for nucleotides)/0.5 (for proteins)). For nucleotides the default scoring matrix used is EDNAFULL and for proteins the default scoring matrix is EBLOSUM62.

It will be clear that whenever nucleotide sequences of RNA molecules are defined by reference to nucleotide sequence of corresponding DNA molecules, the thymine (T) in the nucleotide sequence should be replaced by uracil (U). Whether reference is made to RNA or DNA molecules will be clear from the context of the application.

“Substantially identical” or “essentially similar”, as used herein, refers to sequences, which, when optimally aligned as defined above, share at least a certain minimal percentage of sequence identity (as defined further below).

“Stringent hybridization conditions” can be used to identify nucleotide sequences, which are substantially identical to a given nucleotide sequence. Stringent conditions are sequence dependent and will be different in different circumstances. Generally, stringent conditions are selected to be about 5° C. lower than the thermal melting point (Tₘ) for the specific sequences at a defined ionic strength and pH. The Tₘ is the temperature (under defined ionic strength and pH) at which 50% of the target sequence hybridizes to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridizations (Northern blots using a probe of e.g. 100 nt) are for example those which include at least one wash in 0.2×SSC at 63° C. for 20 min, or equivalent conditions.

“High stringency conditions” can be provided, for example, by hybridization at 65° C. in an aqueous solution containing 6×SSC (20×SSC contains 3.0 M NaCl, 0.3 M Na-citrate, pH 7.0), 5×Denhardt's (100×Denhardt's contains 2% Ficoll, 2% Polyvinyl pyrollidone, 2% Bovine Serum Albumin), 0.5% sodium dodecyl sulphate (SDS), and 20 μg/ml denaturated carrier DNA (single-stranded fish sperm DNA, with an average length of 120-3000 nucleotides) as non-specific competitor. Following hybridization, high stringency washing may be done in several steps, with a final wash (about 30 min) at the hybridization temperature in 0.2-0.1×SSC, 0.1% SDS.

“Moderate stringency conditions” refers to conditions equivalent to hybridization in the above described solution but at about 60-62° C. Moderate stringency washing may be done at the hybridization temperature in 1×SSC, 0.1% SDS.

“Low stringency” refers to conditions equivalent to hybridization in the above described solution at about 50-52° C. Low stringency washing may be done at the hybridization temperature in 2×SSC, 0.1% SDS. See also Sambrook et al. (1989) and Sambrook and Russell (2001).

### DETAILED DESCRIPTION OF THE INVENTION

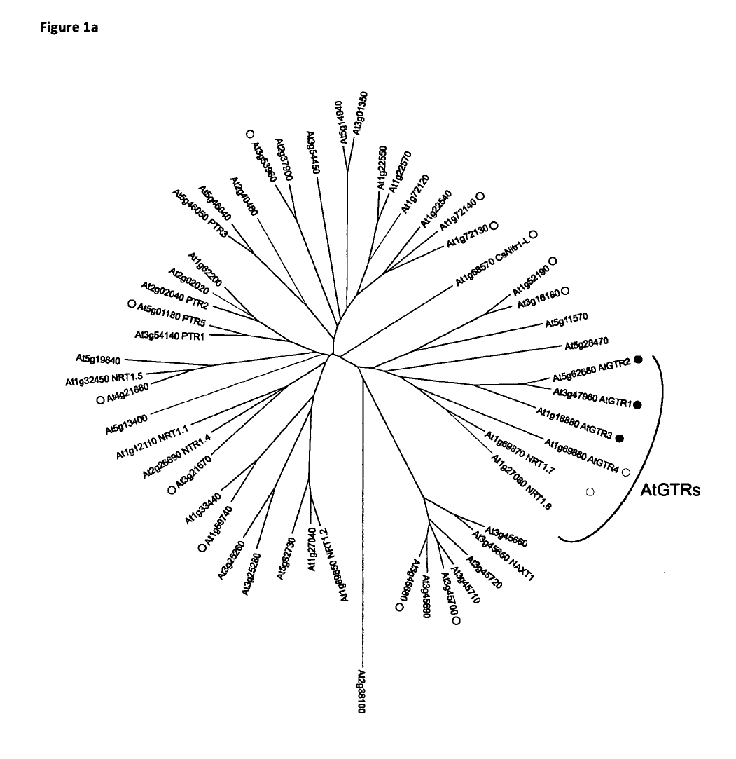

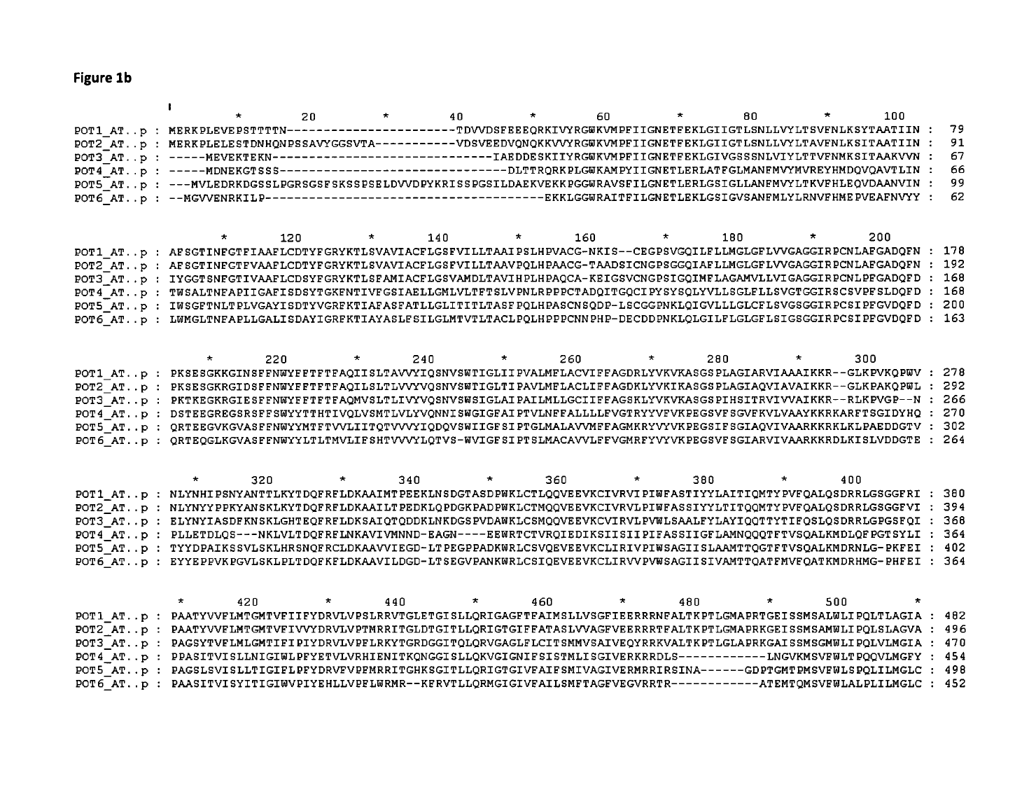

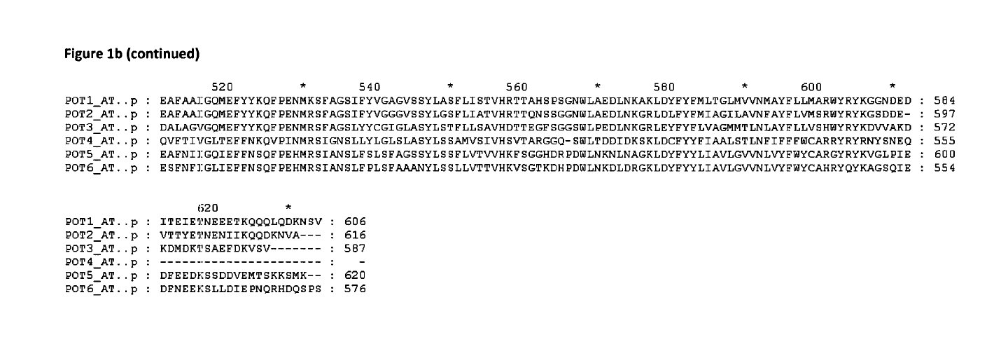

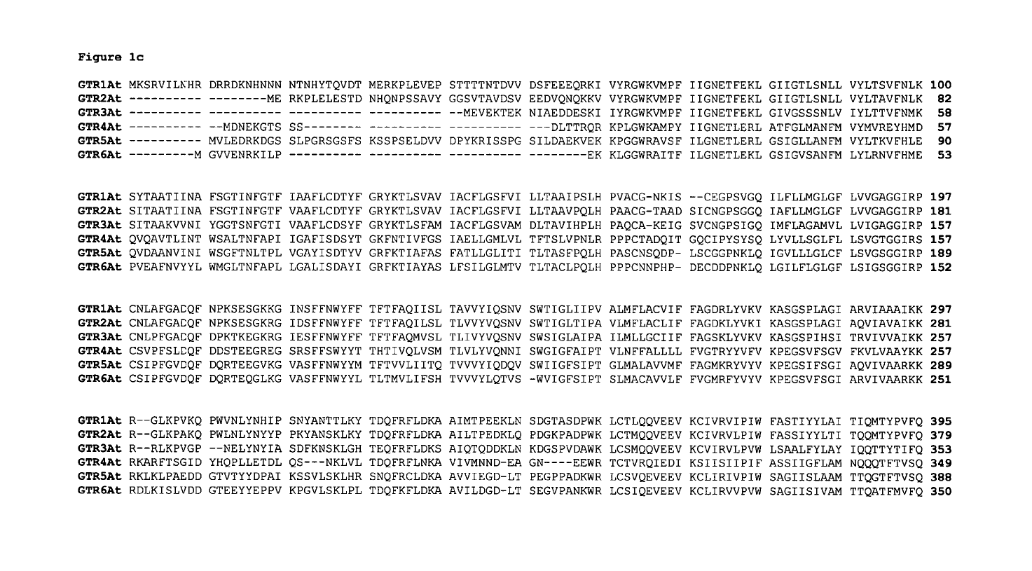

The present invention is based on the identification of GSL transporter (GTR) proteins and corresponding GTR genes in the model plant Arabidopsis thaliana (Nour-Eldin, 2007, supra). It was found by the inventors that the Arabidopsis GTR genes form a small subclade with six homologs (FIG. 1a), named GTR1 to 6. The inventors further found that seeds of Arabidopsis plants knocked out in either GTR1 or GTR2 transporters had no significant reduction and about 50% reduction in total aliphatic GSLs concentrations, respectively, compared to wildtype plants, and that seeds of Arabidopsis plants knocked out in both GTR1 and GTR2 transporters had a zero GSL seed phenotype. Surprisingly, GSLs levels in senescent leaves from gtr knockout plants were high whereas, in wildtype plants, leaves become depleted in GSLs upon aging. The inventors further found that Brassica rapa (genome AA, 2n=20) and Brassica oleracea (genome CC, 2n=18) each comprise three GTR1 genes three GTR2 genes in their genome, while Brassica napus (genome AACC, 2n=4x=38), which is an allotetraploid (amphidiploid) species containing essentially two diploid genomes (the A and the C genome) due to its origin from diploid ancestors, comprises six GTR1 genes and six GTR2 genes in its genome; three of the six GTR1 and GTR2 genes are located on the A genome and three on the C genome. Brassica juncea (genome AABB, 2n=4x=36), which is another allotetraploid species containing essentially two diploid genomes (the A and the B genome) also comprises six GTR2 genes in its genome; three of the six GTR2 are located on the A genome and three on the B genome The GTR1 and GTR2 genes that are located on the A genome are herein referred to as “GTRx-Ay” (wherein x is 1 or 2 and y is 1, 2 or 3) and the GTR1 and GTR2 genes that are located on the C genome are herein referred to as “GTRx-Cy” (wherein x is 1 or 2 and y is 1, 2 or 3). The GTR1 and GTR2 genes that are located on the B genome are herein referred to as “GTRx-By” (wherein x is 1 or 2 and y is 1, 2 or 3). The GTRx-Ay genes from B. napus are said to be “homeologous” to the corresponding GTRx-Cy gene from B. napus, i.e. the “A gene” is found on the A genome and originates from the diploid ancestor B. rapa (AA), while the “C gene” is found on the C genome of B. napus and originates from the diploid ancestor B. oleracea (CC). Similarly, the GTRx-Ay genes from B. juncea are said to be “homeologous” to the corresponding GTRx-By gene from B. juncea, i.e. the “A gene” is found on the A genome and originates from the diploid ancestor B. rapa (AA), while the “B gene” is found on the B genome of B. napus and originates from the diploid ancestor B. nigra (BB)

As in any diploid genome, two “alleles” can be present in vivo for each GTR gene at each GTR locus in the genome (one allele being the gene sequence found on one chromosome and the other on the homologous chromosome). The nucleotide sequence of these two alleles may be identical (homozygous plant) or different (heterozygous plant) in any given plant, although the number of different possible alleles existing for each GTR gene may be much larger than two in the species population as a whole.

Provided herein are nucleic acid sequences of wild type and mutant GTR genes/alleles from Brassicaceae species, as well as the wild type and mutant GTR proteins. Also provided are methods of generating and combining mutant and wild type GTR alleles in Brassicaceae plants, as well as Brassicaceae plants and plant parts comprising specific combinations of wild type and mutant GTR alleles in their genome, whereby the GSL content is modified in specific parts of these plants. The use of these plants for transferring mutant GTR alleles to other plants is also an embodiment of the invention, as are the plant products of any of the plants described. In addition kits and methods for marker assisted selection (MAS) for combining or detecting GTR genes and/or alleles are provided. Different embodiments of the invention are described in detail herein below.

Nucleic Acid Sequences According to the Invention

Provided are both wild type GTR nucleic acid sequences encoding functional GTR proteins and mutant gtr nucleic acid sequences (comprising one or more mutations, preferably mutations which result in no or a significantly reduced GSL transport activity of the encoded GTR protein or in no GTR protein being produced) of GTR genes from Brassicaceae, particularly from Brassica species, especially from Brassica napus, Brassica juncea, Brassica rapa or Brassica oleracea, but also from other Brassica crop species. For example, Brassica species comprising an A and/or a C genome may comprise different alleles of GTR-A or GTR-C genes, which can be identified and combined in a single plant according to the invention. In addition, mutagenesis methods can be used to generate mutations in wild type GTR alleles, thereby generating mutant gtr alleles for use according to the invention. Because specific GTR alleles are preferably combined in a plant by crossing and selection, in one embodiment the GTR and/or gtr nucleic acid sequences are provided within a plant (i.e. endogenously), e.g. a Brassica plant, preferably a Brassica plant which can be crossed with Brassica napus, Brassica juncea, Brassica rapa or Brassica oleracea or which can be used to make a “synthetic” Brassica napus plant. Hybridization between different Brassica species is described in the art, e.g., as referred to in Snowdon (2007, Chromosome research 15: 85-95). Interspecific hybridization can, for example, be used to transfer genes from, e.g., the C genome in B. napus (AACC) to the C genome in B. carinata (BBCC), or even from, e.g., the C genome in B. napus (AACC) to the B genome in B. juncea (AABB) (by the sporadic event of illegitimate recombination between their C and B genomes). “Resynthesized” or “synthetic” Brassica napus lines can be produced by crossing the original ancestors, B. oleracea (CC) and B. rapa (AA). Interspecific, and also intergeneric, incompatibility barriers can be successfully overcome in crosses between Brassica crop species and their relatives, e.g., by embryo rescue techniques or protoplast fusion (see e.g. Snowdon, above).

However, isolated GTR and gtr nucleic acid sequences (e.g. isolated from the plant by cloning or made synthetically by DNA synthesis), as well as variants thereof and fragments of any of these are also provided herein, as these can be used to determine which sequence is present endogenously in a plant or plant part, whether the sequence encodes a functional, a non-functional or no protein (e.g. by expression in a recombinant host cell as described below) and for selection and transfer of specific alleles from one plant into another, in order to generate a plant having the desired combination of functional and mutant alleles.

“GTR nucleic acid sequences” or “GTR variant nucleic acid sequences” according to the invention are nucleic acid sequences encoding an amino acid sequence having at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 55%, at least 56%, at least 57%, at least 58%, at least 59%, at least 60%, at least 61%, at least 62%, at least 63%, at least 64%, at least 65%, at least 70%, at least 75%, at least 76%, at least 77%, at least 78%, at least 79%, at least 80%, at least 85%, at least 90%, at least 95%, 98%, 99% or 100% sequence identity with SEQ ID NO: 2, or nucleic acid sequences having at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 55%, at least 56%, at least 57%, at least 58%, at least 59%, at least 60%, at least 61%, at least 62%, at least 63%, at least 64%, at least 65%, at least 70%, at least 75%, at least 76%, at least 77%, at least 78%, at least 79%, at least 80%, at least 85%, at least 90%, at least 95%, 98%, 99% or 100% sequence identity with SEQ ID NO: 1. These nucleic acid sequences may also be referred to as being “essentially similar” or “essentially identical” to the GTR sequences provided in the sequence listing.

Nucleic acid sequences of GTR1 to 6 have been isolated from Arabidopsis, of GTRx-Ay from B. rapa, B. juncea and from B. napus, of GTRx-By from B. juncea and of GTRx-Cy from B. oleracea and from B. napus as depicted in the sequence listing. The wild type GTR sequences are depicted, while the mutant gtr sequences of these sequences, and of sequences essentially similar to these, are described herein below and in the Examples, with reference to the wild type GTR sequences. The genomic GTR protein-encoding DNA, and corresponding pre-mRNA, comprises 4 exons (numbered exons 1-4 starting from the 5′ end) interrupted by 3 introns (numbered introns 1-3, starting from the 5′ end). In the cDNA and corresponding processed mRNA (i.e. the spliced RNA), introns are removed and exons are joined, as depicted in the sequence listing. Exon sequences are more conserved evolutionarily and are therefore less variable than intron sequences.

“GTR1, 2, 3, 4, 5 or 6 nucleic acid sequences” or “GTR1, 2, 3, 4, 5 or 6 variant nucleic acid sequences” according to the invention are nucleic acid sequences encoding an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, 96%, 97%, 98%, 99% or 100% sequence identity with SEQ ID NO: 2, 4, 6, 8, 10 or 12, respectively, or nucleic acid sequences having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, 96%, 97%, 98%, 99% or 100% sequence identity with SEQ ID NO: 1, 3, 5, 7, 9 or 11. These nucleic acid sequences may also be referred to as being “essentially similar” or “essentially identical” to the GTR sequences provided in the sequence listing.

Thus the invention provides both nucleic acid sequences encoding wild type, functional GTR1, 2, 3, 4, 5 or 6 proteins, including variants and fragments thereof (as defined further below), as well as mutant nucleic acid sequences of any of these, whereby the mutation in the nucleic acid sequence preferably results in one or more amino acids being inserted, deleted or substituted in comparison to the wild type GTR protein. Preferably the mutation(s) in the nucleic acid sequence result in one or more amino acid changes (i.e. in relation to the wild type amino acid sequence one or more amino acids are inserted, deleted and/or substituted) whereby the GSL transport activity of the GTR protein is significantly reduced or completely abolished. A significant reduction in or complete abolishment of the GSL transport activity of the GTR protein refers herein to a reduction in or abolishment of the GSL transport activity of the GTR protein, such that the GSL content is modified in specific parts of a plant expressing the mutant GTR protein as compared to a plant expressing the corresponding wild type GTR protein.

To determine the functionality of specific GTR nucleic acids or proteins, they can, for example, be functionally screened in Xenopus oocytes as described by Nour-Eldin et al. (2006, Plant Methods 2: 17) and in the Examples below.

To determine the functionality of specific GTR alleles or proteins in plants, particularly in Brassicaceae plants, GSL content can, for example, be compared between specific parts of plants comprising different forms of the specific GTR alleles or proteins, for example, mutated and wildtype forms of the specific GTR alleles or proteins, e.g., by performing HPLC-UV analysis of methanol extracts desulfated and eluted from sephadex anion exchange columns as described by, e.g., Hansen et al. (2007, Plant J. 50 (5): 902-910) or as described in the Examples below.

Both endogenous and isolated nucleic acid sequences are provided herein. Also provided are fragments of the GTR sequences and GTR variant nucleic acid sequences defined above, for use as primers or probes and as components of kits according to another aspect of the invention (see further below). A “fragment” of a GTR or gtr nucleic acid sequence or variant thereof (as defined) may be of various lengths, such as at least 10, 12, 15, 18, 20, 50, 100, 200, 500, 600 contiguous nucleotides of the GTR or gtr sequence (or of the variant sequence).

Nucleic Acid Sequences Encoding Functional GTR Proteins

The nucleic acid sequences depicted in the sequence listing encode wild type, functional GTR proteins from Arabidopsis, B. rapa, B. oleracea, B. juncea and B. napus. Thus, these sequences are endogenous to the plants from which they were isolated. Other Brassicaceae plants, including Brassica crop species, varieties, breeding lines or wild accessions, may be screened for other GTR alleles, encoding the same GTR proteins or variants thereof. For example, nucleic acid hybridization techniques (e.g. Southern blot analysis, using for example stringent hybridization conditions) or PCR-based techniques may be used to identify GTR alleles endogenous to other Brassicaceae plants, such as various Brassica napus, oleracea and rapa varieties, lines or accessions, but also Brassica juncea (especially GTR alleles on the A-genome) and Brassica carinata (especially GTR alleles on the C-genome) plants, organs and tissues can be screened for other wild type GTR alleles. To screen such plants, plant organs or tissues for the presence of GTR alleles, the GTR nucleic acid sequences provided in the sequence listing, or variants or fragments of any of these, may be used. For example whole sequences or fragments may be used as probes or primers. For example specific or degenerate primers may be used to amplify nucleic acid sequences encoding GTR proteins from the genomic DNA of the plant, plant organ or tissue. These GTR nucleic acid sequences may be isolated and sequenced using standard molecular biology techniques. Bioinformatics analysis may then be used to characterize the allele(s), for example in order to determine which GTR allele the sequence corresponds to and which GTR protein or protein variant is encoded by the sequence.

Whether a nucleic acid sequence encodes a functional GTR protein can be analyzed by recombinant DNA techniques as known in the art, e.g., by a genetic complementation test using, e.g., an Arabidopsis plant, which is homozygous for one or more full knock-out gtr mutant alleles or a Brassica plant, which is homozygous for one or more full knock-out gtr mutant allele or by functionally screening the GTR protein in Xenopus oocytes as described by Nour-Eldin et al. (2006, Plant Methods 2, 17).

In addition, it is understood that GTR nucleic acid sequences and variants thereof (or fragments of any of these) may be identified in silico, by screening nucleic acid databases for essentially similar sequences. Likewise, a nucleic acid sequence may be synthesized chemically. Fragments of nucleic acid molecules according to the invention are also provided, which are described further below. Fragments include nucleic acid sequences encoding only specific conserved and functional domains as described below.

Nucleic Acid Sequences Encoding Mutant GTR Proteins

Nucleic acid sequences comprising one or more nucleotide deletions, insertions or substitutions relative to the wild type nucleic acid sequences are another embodiment of the invention, as are fragments of such mutant nucleic acid molecules. Such mutant nucleic acid sequences (referred to as gtr sequences) can be generated and/or identified using various known methods, as described further below. Again, such nucleic acid molecules are provided both in endogenous form and in isolated form. In one embodiment, the mutation(s) result in one or more changes (deletions, insertions and/or substitutions) in the amino acid sequence of the encoded GTR protein (i.e. it is not a “silent mutation”). In another embodiment, the mutation(s) in the nucleic acid sequence result in a significantly reduced or completely abolished GSL transport activity of the encoded GTR protein relative to the wild type protein.

The nucleic acid molecules may, thus, comprise one or more mutations, such as:
(a) a “missense mutation” or “substitution mutation”, which is a change in the nucleic acid sequence that results in the substitution of an amino acid for another amino acid;
(b) a “nonsense mutation” or “STOP codon mutation”, which is a change in the nucleic acid sequence that results in the introduction of a premature STOP codon and thus the termination of translation (resulting in a truncated protein); plant genes contain the translation stop codons “TGA” (UGA in RNA), “TAA” (UAA in RNA) and “TAG” (UAG in RNA); thus any nucleotide substitution, insertion, deletion which results in one of these codons to be in the mature mRNA being translated (in the reading frame) will terminate translation;
(c) an “insertion mutation” of one or more amino acids, due to one or more codons having been added in the coding sequence of the nucleic acid;
(d) a “deletion mutation” of one or more amino acids, due to one or more codons having been deleted in the coding sequence of the nucleic acid;
(e) a “frameshift mutation”, resulting in the nucleic acid sequence being translated in a different frame downstream of the mutation. A frameshift mutation can have various causes, such as the insertion, deletion or duplication of one or more nucleotides, but also mutations which affect pre-mRNA splicing (splice site mutations) can result in frameshifts;
(f) a “splice site mutation”, which alters or abolishes the correct splicing of the pre-mRNA sequence, resulting in a protein of different amino acid sequence than the wild type. For example, one or more exons may be skipped during RNA splicing, resulting in a protein lacking the amino acids by the skipped exons. Alternatively, the reading frame may be altered through incorrect splicing, or one or more introns may be retained, or alternate splice donors or acceptors may be generated, or splicing may be initiated at an alternate position (e.g. within an intron), or alternate polyadenylation signals may be generated. Correct pre-mRNA splicing is a complex process, which can be affected by various mutations in the nucleotide sequence of the GTR-encoding gene. In higher eukaryotes, such as plants, the major spliceosome splices introns containing GU at the 5′ splice site (donor site) and AG at the 3′ splice site (acceptor site). This GU-AG rule (or GT-AG rule; see Lewin, Genes VI, Oxford University Press 1998, pp 885-920, ISBN 0198577788) is followed in about 99% of splice sites of nuclear eukaryotic genes, while introns containing other dinucleotides at the 5′ and 3′ splice site, such as GC-AG and AU-AC account for only about 1% and 0.1% respectively. Examples of introns containing GU at the 5′ splice site (donor site) and AG at the 3′ splice site (acceptor site) are indicated in the sequence listing.

As already mentioned, it is desired that the mutation(s) in the nucleic acid sequence preferably result in a mutant protein comprising significantly reduced or no GSL transport activity in vivo or in the production of no protein. Any mutation which results in a protein comprising at least one amino acid insertion, deletion and/or substitution relative to the wild type protein can lead to significantly reduced or no GSL transport activity. It is, however, understood that mutations in certain parts of the protein are more likely to result in a reduced function of the mutant GTR protein, such as mutations leading to truncated proteins, whereby significant portions of conserved or functional domains are lacking.

Conserved and functional domains of the Arabidopsis GTR1, 2, 3, 4, 5 and 6 protein can be found in the Arabidopsis Information Resource (TAIR) database (http://www.arabidopsis.org) under At3g47960 for AtGTR1 (SEQ ID NO: 2), At5g62680 for AtGTR2 (SEQ ID NO: 4), At1g18880 for AtGTR3 (SEQ ID NO: 6), At1g69860 for AtGTR4 (SEQ ID NO: 8), At1g69870 for AtGTR5 (SEQ ID NO: 10) and At1g27080 for AtGTR6 (SEQ ID NO: 12) and/or by optimally aligning the Arabidopsis GTR1, 2, 3, 4, 5 and 6 protein sequences and determining conserved domains (see FIG. 1), such as amino acid sequence F/VALTKPTLGM/LAPRKGE/AISS (SEQ ID NO: 2 from the amino acid at position 448 to 466 or SEQ ID No. 142 from amino acid position 478 to 496) and sequences essentially similar thereto (such as SEQ ID NO: 4 from the amino acid at position 462 to 480 and in SEQ ID NO: 6 from the amino acid at position 436 to 454); amino acid sequence L/INxxxLDxY/FY/FxxxxxxxxxNxxY/F (SEQ ID NO: 2 from the amino acid at position 545 to 567 or SEQ ID No. 142 from position 575 to 597) and sequences essentially similar thereto.

Corresponding conserved and functional domains can be determined for the Brassica GTR proteins by optimally aligning the Brassica and Arabidopsis GTR proteins and based on the annotation information in the TAIR database.

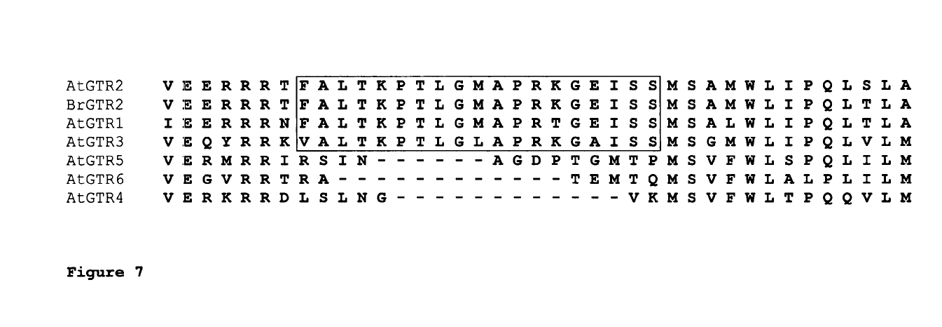

It has been found that Arabidopsis GTR1, 2 and GTR3 contain the above mentioned sequence F/VALTKPTLGM/LAPRKGE/AISS, while Arabidopsis GTR4, 5 and GTR6 do not contain that sequence (see alignment of FIG. 7). As elaborated below in the Examples, GTR1, GTR2 and GTR3 exhibited most uptake activity of glucosinolates in the Xenopus oocytes test. The protein structure of GTR2 protein as predicted contains 12 transmembrane domains (FIG. 8). The conserved sequence forms a GTR1/2/3 specific loop near the carboxyterminus of the protein, as indicated by a dashed arrow in FIG. 8.

A similar domain can be found in GTR1 and GTR2 proteins of Brassica species including BnGTR1-A1 (SEQ ID NO: 14 amino acid positions 456-474); BnGTR1-A2 (SEQ ID NO: 16 amino acid positions 459-477); BnGTR1-A3 (SEQ ID NO: 18 amino acid positions 457-475); BnGTR1-C1 (SEQ ID NO: 20 amino acid positions 456-474); BnGTR1-C2 (SEQ ID NO: 22 amino acid positions 459-477); BnGTR1-C3 (SEQ ID NO: 24 amino acid positions 457-475); BnGTR2-A1 (SEQ ID NO: 26 amino acid positions 458-476); BnGTR2-A2 (SEQ ID NO: 28 amino acid positions 458-476); BnGTR2-A3 (SEQ ID NO: 30 amino acid positions 458-476); BnGTR2-C1 (SEQ ID NO: 32 amino acid positions 458-476); BnGTR2-C2 (SEQ ID NO: 34 amino acid positions 401-419); BnGTR2-C3 (SEQ ID NO: 36 amino acid positions 457-475); BrGTR1-A1 (SEQ ID NO: 38 amino acid positions 456-474); BrGTR1-A2 (SEQ ID NO: 40 amino acid positions 459-477); BrGTR1-A3 (SEQ ID NO: 42 amino acid positions 457-475); BrGTR2-A1 (SEQ ID NO: 44 amino acid positions 458-476); BrGTR2-A2 (SEQ ID NO: 46 amino acid positions 458-476); BrGTR2-A3 (SEQ ID NO: 48 amino acid positions 458-476); BoGTR1-C1 (SEQ ID NO: 50 amino acid positions 456-474); BoGTR1-C2 (SEQ ID NO: 52 amino acid positions 459-477); BoGTR1-C3 (SEQ ID NO: 54 amino acid positions 457-475); BoGTR2-C1 (SEQ ID NO: 56 amino acid positions 458-476); BoGTR2-C2 (SEQ ID NO: 58 amino acid positions 458-476); BoGTR2-C3 (SEQ ID NO: 60 amino acid positions 458-476); BjGTR2-A1 (SEQ ID NO: 120 amino acid positions 458-476); BjGTR2-A2 (SEQ ID NO: 122 amino acid positions 458-476); BjGTR2-A3 (SEQ ID NO: 124 amino acid positions 458-476); BjGTR2-B1 (SEQ ID NO: 126 amino acid positions 405-423); BjGTR2-B2 (SEQ ID NO: 128 amino acid positions 458-476) and BjGTR2-B3 (SEQ ID NO: 130 amino acid positions 452-470).

It has also been recently found, and confirmed by proteomic data, that the GTR1 protein of Arabidopsis thaliana may comprise an aminoterminal peptide extension with a length of 30 amino acids when compared with the GTR1 protein as represented in SEQ ID No 2. The variant with the N-terminal extension is provided in SEQ ID No. 142 and the nucleotide sequence encoding such a protein is provided in SEQ ID No. 141. A similar N-terminal peptide could be found in B. rapa GTR1_A1, GTR1_A2, GTR1_A3, B. napus GTR1_A1, GTR1_A2, GTR1_A3, in B. oleracea GTR_C1, GT1_C2, GTR1_C3 and in B. napus GTR_C1, GT1_C2, GTR1_C3 (SEQ ID NOS: 143-150). It can be expected that a similar extension can be found in the GTR1_B1, GTR1_B2 and GTR1_B3 such as present in B. juncea. Mutations altering this 30/23 amino acid sequence may result in mutant GTR1 protein being located at a different subcellular location in the plant cell, and thus in functionally less or not active GTR1 protein. The amino terminal peptide may also interact with proteins providing a regulatory mechanism impacting the GTR1 activity under certain conditions such as e.g. different kinds of stresses, and mutants in this region may impact functionality also in this manner.

Thus in one embodiment, nucleic acid sequences comprising one or more of any of the types of mutations described above are provided. In another embodiment, gtr sequences comprising one or more stop codon (nonsense) mutations, one or more substitution (missense) mutations and/or one or more splice site (frameshift) mutations are provided. Any of the above mutant nucleic acid sequences are provided per se (in isolated form), as are plants and plant parts comprising such sequences endogenously. In the tables herein below the most preferred gtr alleles are described.

A nonsense mutation in a GTR allele, as used herein, is a mutation in a GTR allele whereby one or more translation stop codons are introduced into the coding DNA and the corresponding mRNA sequence of the corresponding wild type GTR allele. Translation stop codons are TGA (UGA in the mRNA), TAA (UAA) and TAG (UAG). Thus, any mutation (deletion, insertion or substitution) that leads to the generation of an in-frame stop codon in the coding sequence will result in termination of translation and truncation of the amino acid chain. In one embodiment, a mutant GTR allele comprising a nonsense mutation is a GTR allele wherein an in-frame stop codon is introduced in the GTR codon sequence by a single nucleotide substitution, such as the mutation of CAG to TAG, TGG to TAG or TGA, CGA to TGA, CAA to TAA, etc. (see Tables below). In one aspect, a mutant GTR allele comprising a nonsense mutation is a GTR allele comprising a STOP codon at a position corresponding to the codon of the amino acid at position 229 in SEQ ID NO: 66, such as the GTR allele indicated in Table 7 of Example 3 below. In another embodiment, a mutant GTR allele comprising a nonsense mutation is a GTR allele wherein an in-frame stop codon is introduced in the GTR codon sequence by double nucleotide substitutions, such as the mutation of CAG to TAA, TGG to TAA, CGA to TAA, etc. (see Tables below). In yet another embodiment, a mutant GTR allele comprising a nonsense mutation is a GTR allele wherein an in-frame stop codon is introduced in the GTR codon sequence by triple nucleotide substitutions, such as the mutation of CGG to TAA, etc. (see Tables below). The truncated protein lacks the amino acids encoded by the coding DNA downstream of the mutation (i.e. the C-terminal part of the GTR protein) and maintains the amino acids encoded by the coding DNA upstream of the mutation (i.e. the N-terminal part of the GTR protein). The more truncated the mutant GTR protein is in comparison to the wild type GTR protein, the more the truncation may result in a significantly reduced or no activity of the GTR protein.

The Tables herein below describe a range of possible nonsense mutations in Arabidopsis and Brassica GTR sequences provided herein:

TABLE 1
Potential EMS-indiced STOP codon mutations in exon
(abbreviated as E) 1, 2, 3 and 4 of AtGTR1 and AtGTR2

| | AtGTR1 | | | | AtGTR2 | | | |
|---|---|---|---|---|---|---|---|---|
| | | Amino | | | | Amino | | |
| | | acid | | | | acid | | |
| | Nucleotide | position | | | Nucleotide | position | | |
| | position in | in SEQ | | | position in | in SEQ | | |
| | SEQ ID NO: | ID NO: | Wildtype | → Potential | SEQ ID | ID NO: | Wildtype | → Potential |
| | 61 | 62 | codon | stop codons | NO: 63 | 64 | codon | stop codons |
| E1 | 79-81 | 27 | CAG | TAG TAA | 43-45 | 15 | CAG | TAA TAG |
| | 103-105 | 35 | TGG | TAG TAA TGA | 109-111 | 37 | CAG | TAA TAG |
| | | | | | 115-117 | 39 | CAG | TAA TAG |
| | | | | | 139-141 | 47 | TGG | TAA TAG TGA |
| E2 | | | | | 708-710 | 114 | CGA | TGA TAA |
| E3 | 1085-1087 | 147 | CAG | TAG TAA | 877-879 | 140 | CAA | TAA |
| | 1172-1174 | 176 | CAG | TAG TAA | 940-942 | 161 | CAG | TAG TAA |
| | 1226-1228 | 194 | TGG | TGA TAG TAA | 1027-1029 | 190 | CAG | TAA TAG |
| | 1253-1255 | 203 | CAG | TAA TAG | 1081-1083 | 208 | TGG | TGA TAG TAA |
| | 1286-1288 | 214 | CAG | TAG TAA | 1108-1110 | 217 | CAG | TAG TAA |
| | 1301-1303 | 219 | TGG | TAG TAA TGA | 1141-1143 | 228 | CAG | TAG TAA |
| | 1448-1450 | 268 | CGA | TAA TGA | 1156-1158 | 233 | TGG | TAG TGA TAA |
| | 1469-1471 | 275 | CAG | TAA TAG | 1276-1278 | 273 | CAA | TAA |
| | 1475-1477 | 277 | TGG | TAG TGA TAA | 1324-1326 | 289 | CAG | TAA TAG |
| | 1538-1540 | 298 | CAG | TAG TAA | 1330-1332 | 291 | TGG | TAA TGA TAG |
| | | | | | 1393-1395 | 312 | CAA | TAA |
| E4 | 1705-1707 | 324 | TGG | TGA TAG TAA | 1527-1529 | 329 | CAG | TAA TAG |
| | 1723-1725 | 330 | CAG | TAA TAG | 1554-1556 | 338 | TGG | TGA TAG TAA |
| | 1726-1728 | 331 | CAA | TAA | 1572-1574 | 344 | CAA | TAA |
| | 1768-1770 | 345 | TGG | TAG TGA TAA | 1575-1577 | 345 | CAA | TAA |
| | 1807-1809 | 358 | CAA | TAA | 1617-1619 | 359 | TGG | TGA TAA TAG |
| | 1828-1830 | 365 | CAA | TAA | 1653-1655 | 371 | CAA | TAA |
| | 1837-1839 | 368 | CAG | TAG TAA | 1656-1658 | 372 | CAA | TAA |
| | 1846-1848 | 371 | CGA | TAA TGA | 1677-1679 | 379 | CAA | TAA |
| | 1849-1851 | 372 | CGA | TGA TAA | 1686-1688 | 382 | CAG | TAG TAA |
| | 1999-2001 | 422 | CAA | TAA | 1848-1850 | 436 | CAG | TAA TAG |
| | 2113-2115 | 460 | CGG | TGA TAA TAG | 1914-1916 | 458 | CGA | TGA TAA |
| | 2146-2148 | 471 | TGG | TAA TGA TAG | 1995-1997 | 485 | TGG | TGA TAG TAA |
| | 2158-2160 | 475 | CAG | TAG TAA | 2007-2009 | 489 | CAG | TAA TAG |
| | 2203-2205 | 490 | CAA | TAA | 2052-2054 | 504 | CAA | TAA |
| | 2224-2226 | 497 | CAG | TAA TAG | 2073-2075 | 511 | CAG | TAG TAA |
| | 2323-2325 | 530 | CGA | TGA TAA | 2181-2183 | 547 | CAG | TAA TAG |
| | 2353-2355 | 540 | TGG | TAA TAG TGA | 2202-2204 | 554 | TGG | TGA TAA TAG |
| | 2455-2457 | 574 | TGG | TAA TAG TGA | 2304-2306 | 588 | TGG | TAA TGA TAG |
| | 2524-2526 | 597 | CAA | TAA | 2370-2372 | 610 | CAG | TAA TAG |
| | 2527-2529 | 598 | CAG | TAG TAA | 2373-2375 | 611 | CAA | TAA |
| | 2530-2532 | 599 | CAG | TAA TAG | | | | |
| | 2536-2538 | 601 | CAA | TAA | | | | |

For AtGTR1 the numbers may be increased with 90 nt or 30 AA to make reference to the position taking into account the N-terminal extension of 30 AA.

TABLE 2a
Potential EMS-induced STOP codon mutations in exon
(abbreviated as E) 1, 2, 3 and 4 o BnGTR1-A1 and C1

| | BnGTR1-A1 | | | BnGTR1-C1 | | |
|---|---|---|---|---|---|---|
| | Nucleotide | | | Nucleotide | | |
| | position in | | | position in | | |
| | SEQ ID NO: | Wildtype | → Potential | SEQ ID NO: | Wildtype | → Potential |
| | 13* | codon | stop codons | 19* | codon | stop codons |
| E1 | 103-105 | CAG | TAG TAA | 103-105 | CAG | TAA TAG |
| | 127-129 | TGG | TAA TAG TGA | 127-129 | TGG | TAG TAA TGA |
| E3 | 991-993 | CAG | TAA TAG | 992-994 | CAG | TAG TAA |
| | 1003-1005 | CAA | TAA | 1004-1006 | CAA | TAA |
| | 1021-1023 | CAG | TAA TAG | 1022-1024 | CAG | TAG TAA |
| | 1108-1110 | CAG | TAA TAG | 1109-1111 | CAG | TAA TAG |
| | 1162-1164 | TGG | TAA TAG TGA | 1163-1165 | TGG | TGA TAG TAA |
| | 1189-1191 | CAG | TAA TAG | 1190-1192 | CAG | TAG TAA |
| | 1222-1224 | CAG | TAA TAG | 1223-1225 | CAG | TAA TAG |
| | 1237-1239 | TGG | TGA TAA TAG | 1238-1240 | TGG | TAG TGA TAA |
| | 1405-1407 | CAG | TAG TAA | 1406-1408 | CAG | TAG TAA |
| | 1411-1413 | TGG | TAA TGA TAG | 1412-1414 | TGG | TGA TAG TAA |
| | 1474-1476 | CAG | TAG TAA | 1475-1477 | CAG | TAA TAG |
| E4 | 1632-1634 | TGG | TAA TAG TGA | 1633-1635 | TGG | TAA TGA TAG |
| | 1650-1652 | CAG | TAA TAG | 1651-1653 | CAG | TAA TAG |
| | 1695-1697 | TGG | TAA TGA TAG | 1696-1698 | TGG | TAA TAG TGA |
| | 1734-1736 | CAA | TAA | 1735-1737 | CAA | TAA |
| | 1755-1757 | CAA | TAA | 1756-1758 | CAA | TAA |
| | 1764-1766 | CAG | TAA TAG | 1765-1767 | CAG | TAA TAG |
| | 1776-1778 | CGA | TGA TAA | 1777-1779 | CTA | |
| | 1926-1928 | CAA | TAA | 1927-1929 | CAA | TAA |
| | 2016-2018 | CGA | TAA TGA | 2017-2019 | CGA | TAA TGA |
| | 2040-2042 | CGA | TAA TGA | 2041-2043 | CGA | TGA TAA |
| | 2073-2075 | TGG | TGA TAA TAG | 2074-2076 | TGG | TAG TAA TGA |
| | 2085-2087 | CAG | TAA TAG | 2086-2088 | CAG | TAG TAA |
| | 2130-2132 | CAA | TAA | 2131-2133 | CAA | TAA |
| | 2151-2153 | CAG | TAA TAG | 2152-2154 | CAG | TAA TAG |
| | 2280-2282 | TGG | TAG TAA TGA | 2281-2283 | TGG | TAG TAA TGA |
| | 2382-2384 | TGG | TAG TAA TGA | 2383-2385 | TGG | TAA TAG TGA |
| | 2451-2453 | CAA | TAA | 2452-2454 | CAA | TAA |
| | 2454-2456 | CAA | TAA | 2455-2457 | CAA | TAA |

*The corresponding amino acid position in SEQ ID NO: 14 and 20 corresponds to the number of the amino acid encoded by the amino acid codon at the indicated nucleotide positions in SEQ ID NO: 13 and 19, respectively, in the sequence listing.

For BnGTR1-C1 of SEQ ID No. 137 stopcodons can be induced by EMS mutagenesis at each of the following position: 103-105, 127-129, 1227-1229, 1239-1241, 1257-1259, 1344-1346, 1398-1400, 1425-1427, 1458-1460, 1473-1475, 1641-1643, 1647-1649, 1710-1712, 1868-1870, 1886-1888, 1931-1933, 1970-1972, 1991-1993, 2000-2002, 2162-2164, 2252-2254, 2276-2278, 2309-2311, 2321-2323, 2366-2368, 2387-2389, 2516-2518, 2618-2620, 2687-2689, 2690-2692

TABLE 2b
Potential EMS-induced STOP codon mutations in exon
(abbreviated as E) 1, 2, 3 and 4 of BnGTR1-A2 and C2

| | BnGTR1-A2 | | | BnGTR1-C2 | | |
|---|---|---|---|---|---|---|
| | Nucleotide | | | Nucleotide | | |
| | position in | | | position in | | |
| | SEQ ID NO: | Wildtype | → Potential | SEQ ID NO: | Wildtype | → Potential |
| X | 15 | codon | stop codons | 21 | codon | stop codons |
| E1 | 49-51 | CAG | TAG TAA | 49-51 | CAG | TAA TAG |
| | 112-114 | CAG | TAG TAA | 112-114 | CAG | TAA TAG |
| | 136-138 | TGG | TGA TAA TAG | 136-138 | TGG | TAA TGA TAG |
| E2 | 730-732 | CAA | TAA | 703-705 | CAA | TAA |
| E3 | 1068-1070 | CAG | TAG TAA | 1048-1050 | CAG | TAA TAG |
| | 1155-1157 | CAG | TAG TAA | 1135-1137 | CAG | TAG TAA |
| | 1209-111 | TGG | TAA TAG TGA | 1189-1191 | TGG | TGA TAA TAG |
| | 1236-1238 | CAG | TAG TAA | 1216-1218 | CAG | TAA TAG |
| | 1269-1271 | CAG | TAG TAA | 1249-1251 | CAG | TAG TAA |
| | 1284-1286 | TGG | TAG TGA TAA | 1264-1266 | TGG | TGA TAG TAA |
| | 1431-1433 | CGA | TGA TAA | 1411-1413 | CGA | TAA TGA |
| | 1452-1454 | CAG | TGA TAA | 1432-1434 | CAG | TAG TAA |
| | 1458-1460 | TGG | TGA TAA TAG | 1438-1440 | TGG | TGA TAA TAG |
| | 1521-1523 | CAG | TAA TAG | 1501-1503 | CAG | TAG TAA |
| E4 | 1823-1825 | TGG | TAA TAG TGA | 1778-1780 | TGG | TAA TAG TGA |
| | 1841-1843 | CAG | TAG TAA | 1796-1798 | CAG | TAG TAA |
| | 1844-1846 | CAA | TAA | 1799-1801 | CAA | TAA |
| | 1886-1888 | TGG | TAA TAG TGA | 1841-1843 | TGG | TGA TAA TAG |
| | 1925-1927 | CAA | TAA | 1880-1882 | CAA | TAA |
| | 1946-1948 | CAA | TAA | 1901-1903 | CAA | TAA |
| | 1952-1954 | CAA | TAA | 1907-1909 | CAA | TAA |
| | 1955-1957 | CAG | TAA TAG | 1910-1912 | CAG | TAA TAG |
| | 2117-2119 | CAA | TAA | 2072-2074 | CAA | TAA |
| | 2210-2212 | CAG | TAA TAG | 2165-2167 | CAG | TAG TAA |
| | 2231-2233 | CGA | TGA TAA | 2186-2188 | CGA | TGA TAA |
| | 2264-2266 | TGG | TAA TGA TAG | 2219-2221 | TGG | TAG TGA TAA |
| | 2276-2278 | CAG | TAA TAG | 2231-2233 | CAG | TAA TAG |
| | 2321-2323 | CAG | TAA TAG | 2276-2278 | CAG | TAA TAG |
| | 2342-2344 | CAG | TAA TAG | 2297-2299 | CAG | TAG TAA |
| | 2441-2443 | CGG | TAA TGA TAG | 2396-2398 | AGG | |
| | 2471-2473 | TGG | TGA TAG TAA | 2426-2428 | TGG | TAA TGA TAG |
| | 2642-2644 | CAG | TAA TAG | 2597-2599 | CAG | TAG TAA |
| | 2648-2650 | CAA | TAA | 2603-2605 | CAA | TAA |

*The corresponding amino acid position in SEQ ID NO: 16 and 22 corresponds to the number of the amino acid encoded by the amino acid codon at the indicated nucleotide positions in SEQ ID NO: 15 and 21, respectively, in the sequence listing.

TABLE 2c
Potential EMS-induced STOP codon mutations in exon
(abbreviated as E) 1, 2, 3 and 4 of BnGTR1-A3 and C3

| | BnGTR1-A3 | | | BnGTR1-C3 | | |
|---|---|---|---|---|---|---|
| | Nucleotide | | | Nucleotide | | |
| | position in | | | position in | | |
| | SEQ ID NO: | Wildtype | Potential | SEQ ID NO: | Wildtype | Potential |
| | 17 | codon | stop codons | 23 | codon | stop codons |
| E1 | 106-108 | CAG | TAA TAG | 106-108 | CAG | TAA TAG |
| | 130-132 | TGG | TAA TGA TAG | 130-132 | TGG | TGA TAG TAA |
| E2 | 579-581 | CAA | TAA | 581-583 | CAA | TAA |
| E3 | 886-888 | CAA | TAA | 887-889 | CAA | TAA |
| | 904-906 | CAG | TAG TAA | 905-907 | CAG | TAG TAA |
| | 991-993 | CAG | TAG TAA | 992-994 | CAG | TAG TAA |
| | 1045-1047 | TGG | TGA TAG TAA | 1046-1048 | TGG | TAA TGA TAG |
| | 1072-1074 | CAG | TAG TAA | 1073-1075 | CAG | TAG TAA |
| | 1105-1107 | CAG | TAA TAG | 1106-1108 | CAG | TAG TAA |
| | 1120-1122 | TGG | TAG TAA TGA | 1121-1123 | TGG | TGA TAA TAG |
| | 1267-1269 | CGA | TAA TGA | 1268-1270 | CGA | TGA TAA |
| | 1357-1359 | CAG | TAA TAG | 1358-1360 | CAG | TAG TAA |
| E4 | 1503-1505 | TGG | TAA TAG TGA | 1512-1514 | TGG | TAA TAG TGA |
| | 1521-1523 | CAG | TAA TAG | 1530-1532 | CAG | TAA TAG |
| | 1524-1526 | CAG | TAA TAG | 1533-1535 | CAG | TAA TAG |
| | 1566-1568 | TGG | TAG TGA TAA | 1575-1577 | TGG | TAA TGA TAG |
| | 1605-1607 | CAA | TAA | 1614-1616 | CAA | TAA |
| | 1626-1628 | CAA | TAA | 1635-1637 | CAA | TAA |
| | 1635-1637 | CAG | TAA TAG | 1644-1646 | CAG | TAA TAG |
| | 1644-1646 | CGG | TGA TAA TAG | 1653-1655 | CGG | TAA TGA TAG |
| | 1797-1799 | CAA | TAA | 1806-1808 | CAA | TAA |
| | 1863-1865 | CGG | TAG TGA TAA | 1872-1874 | CGG | TAA TGA TAG |
| | 1866-1868 | CGA | TGA TAA | 1875-1877 | CGA | TGA TAA |
| | 1911-1913 | CGA | TGA TAA | 1920-1922 | CGA | TAA TGA |
| | 1944-1946 | TGG | TAG TAA TGA | 1953-1955 | TGG | TAG TGA TAA |
| | 1956-1958 | CAG | TAA TAG | 1965-1967 | CAG | TAG TAA |
| | 2001-2003 | CAA | TAA | 2010-2012 | CAA | TAA |
| | 2022-2024 | CAG | TAG TAA | 2031-2033 | CAG | TAA TAG |
| | 2151-2153 | TGG | TGA TAA TAG | 2160-2162 | TGG | TAA TAG TGA |
| | 2328-2330 | CAG | TAG TAA | 2337-2339 | CAG | TAA TAG |
| | 2331-2333 | CAA | TAA | 2340-2342 | CAA | TAA |

*The corresponding amino acid position in SEQ ID NO: 18 and 24 corresponds to the number of the amino acid encoded by the amino acid codon at the indicated nucleotide positions in SEQ ID NO: 17 and 23, respectively, in the sequence listing.

TABLE 3a
Potential EMS-induced STOP codon mutations in exon
(abbreviated as E) 1, 2, 3 and 4 of BnGTR2-A1 and C1

| | BnGTR2-A1 | | | BnGTR2-C1 | | |
|---|---|---|---|---|---|---|
| | Nucleotide | | | Nucleotide | | |
| | position in | | | position in | | |
| | SEQ ID NO: | Wildtype | → Potential | SEQ ID NO: | Wildtype | → Potential |
| | 25 | codon | stop codons | 31 | codon | stop codons |
| E1 | 40-42 | CAA | TAA | 40-42 | CAA | TAA |
| | 133-135 | TGG | TGA TAA TAG | 133-135 | TGG | TGA TAA TAG |
| E2 | 675-677 | CGA | TGA TAA | 702-704 | CGA | TAA TAG |
| E3 | 848-850 | CAA | TAA | 872-874 | CAA | TAA |
| | 905-907 | CAG | TAA TAG | 929-931^{b} | CAG | TAG TAA |
| | 992-994 | CAG | TAA TAG | 1016-1018 | CAG | TAA TAG |
| | 1022-1024 | CGA | TAA TGA | 1046-1048 | CGA | TAA TGA |
| | 1046-1048 | TGG | TGA TAG TAA | 1070-1072 | TGG | TGA TAA TAG |
| | 1073-1075 | CAG | TAA TAG | 1097-1099 | CAG | TAG TAA |
| | 1106-1108 | CAG | TAA TAG | 1130-1132 | CAG | TAG TAA |
| | 1121-1123 | TGG | TAA TGA TAG | 1145-1147^{c} | TGG | TAG TGA TAA |
| | 1241-1243^{a} | CAA | TAA | 1265-1267 | CAA | TAA |
| | 1295-1297 | TGG | TGA TAA TAG | 1319-1321 | TGG | TAG TAA TGA |
| | 1358-1360 | CAG | TAA TAG | 1382-1384 | CAG | TAG TAA |
| E4 | 1535-1537 | TGG | TAG TAA TGA | 1554-1556 | TGG | TAA TGA TAG |
| | 1553-1555 | CAA | TAA | 1572-1574 | CAA | TAA |
| | 1556-1558 | CAA | TAA | 1575-1577 | CAA | TAA |
| | 1598-1600 | TGG | TAA TGA TAG | 1617-1619 | TGG | TAA TGA TAG |
| | 1634-1636 | CAA | TAA | 1653-1655 | CAA | TAA |
| | 1637-1639 | CAA | TAA | 1656-1658 | CAA | TAA |
| | 1659-1660 | CAA | TAA | 1677-1679 | CAA | TAA |
| | 1667-1669 | CAG | TAG TAA | 1686-1688 | CAG | TAA TAG |
| | 1829-1831 | CAG | TAA TAG | 1848-1850 | CAG | TAG TAA |
| | 1895-1897 | CGG | TAG TAA TGA | 1914-1916 | CGG | TAA TGA TAG |
| | 1976-1978 | TGG | TGA TAA TAG | 1995-1997 | TGG | TAG TGA TAA |
| | 1988-1990 | CAA | TAA | 2007-2009 | CAA | TAA |
| | 2033-2035 | CAG | TAA TAG | 2052-2054 | CAG | TAG TAA |
| | 2054-2056 | CAG | TAA TAG | 2073-2075 | CAA | TAA |
| | 2162-2164 | CAG | TAA TAG | 2181-2183 | CAG | TAA TAG |
| | 2183-2185 | TGG | TGA TAA TAG | 2202-2204 | TGG | TAA TGA TAG |
| | 2285-2287 | TGG | TGA TAA TAG | 2304-2306 | TGG | TAA TGA TAG |
| | 2351-2353 | CAA | TAA | 2370-2372 | CAA | TAA |
| | 2354-2356 | CAA | TAA | 2373-2375 | CAA | TAA |

*The corresponding amino acid position in SEQ ID NO: 26 and 32 corresponds to the number of the amino acid encoded by the amino acid codon at the indicated nucleotide positions in SEQ ID NO: 25 and 31, respectively, in the sequence listing.
^{a}BnGTR2-A1-ems02 allele of the examples
^{b}BnGTR2-C1-ems01 allele of the examples
^{c}BnGTR2-C1-ems05 allele of the examples

TABLE 3b
Potential EMS-induced STOP codon mutations in exon
abbreviated as E) 1, 2, 3 and 4 of BnGTR2-A2 and C2

| | BnGTR2-A2 | | | BnGTR2-C2 | | |
|---|---|---|---|---|---|---|
| | Nucleotide | | | Nucleotide | | |
| | position in | | | position in | | |
| | SEQ ID NO: | Wildtype | → Potential | SEQ ID NO: | Wildtype | → Potential |
| x | 27 | codon | stop codons | 33 | codon | stop codons |
| E1 | 40-42 | CAG | TAA TAG | | | |
| | 106-108 | CAG | TAA TAG | | | |
| | 133-135 | TGG | TGA TAG TAA | | | |
| E3 | 870-872^{d} | CAA | TAA | 333-335 | CAA | TAA |
| | 927-929 | CAG | TAA TAG | 390-392 | CAG | TAG TAA |
| | 1014-1016 | CAG | TGA TAA | 477-479 | CAG | TAG TAA |
| | 1068-1070 | TGG | TGA TAA TAG | 531-533 | TGG | TAG TAA TGA |
| | 1095-1097 | CAG | TAG TAA | 558-560 | CAG | TAA TAG |
| | 1128-1130 | CAG | TAG TAA | 591-593 | CAG | TAA TAG |
| | 1143-1145 | TGG | TGA TAA TAG | 606-608 | TGG | TAA TGA TAG |
| | 1263-1265 | CAA | TAA | 726-728 | CAA | TAA |
| | 1311-1313 | CAG | TAA TAG | 774-776 | CAG | TAA TAG |
| | 1317-1319 | TGG | TAA TGA TAG | 780-782^{f} | TGG | TGA TAA TAG |
| | 1380-1382^{e} | CAG | TAG TAA | 843-845 | CAG | TAG TAA |
| E4 | 1532-1534 | TGG | TAG TGA TAA | 994-996 | TGG | TGA TAA TAG |
| | 1550-1552 | CAA | TAA | 1012-1014 | CAA | TAA |
| | 1553-1555 | CAA | TAA | 1015-1017 | CAA | TAA |
| | 1595-1597 | TGG | TGA TAA TAG | 1057-1059 | TGG | TAA TGA TAG |
| | 1631-1633 | CAA | TAA | 1093-1095 | CAA | TAA |
| | 1634-1636 | CAA | TAA | 1096-1098 | CAA | TAA |
| | 1655-1657 | CAA | TAA | 1117-1119 | CAA | TAA |
| | 1664-1666 | CAG | TAA TAG | 1126-1128 | CAG | TAG TAA |
| | 1826-1828 | CAA | TAA | 1288-1290 | CAA | TAA |
| | 1892-1894 | CGG | TAA TAG TGA | 1354-1356 | CGG | TGA TAA TAG |
| | 1940-1942 | CGG | TAG TGA TAA | 1402-1404 | CGG | TGA TAG TAA |
| | 1973-1975 | TGG | TGA TAG TAA | 1435-1437 | TGG | TAG TAA TGA |
| | 1985-1987 | CAG | TAG TAA | 1447-1449 | CAG | TAA TAG |
| | 2030-2032 | CAG | TAG TAA | 1492-1494 | CAG | TAA TAG |
| | 2051-2053 | CAG | TAA TAG | 1513-1515 | CAG | TAG TAA |
| | 2159-2161 | CAG | TAG TAA | 1621-1623 | CAG | TAG TAA |
| | 2180-2182 | TGG | TAG TAA TGA | 1642-1644 | TGG | TGA TAA TAG |
| | 2282-2284 | TGG | TGA TAA TAG | 1744-1746 | TGG | TAA TAG TGA |
| | 2348-2350 | CAA | TAA | 1810-1812 | CAA | TAA |
| | 2351-2353 | CAA | TAA | 1813-1815 | CAA | TAA |

*The corresponding amino acid position in SEQ ID NO: 28 and 34 corresponds to the number of the amino acid encoded by the amino acid codon at the indicated nucleotide positions in SEQ ID NO: 27 and 33, respectively, in the sequence listing.
^{a}BnGTR2-A2-ems03 allele of the examples
^{b}BnGTR2-A2-ems09 allele of the examples
^{c}BnGTR2-C2-ems02 allele of the examples

TABLE 3c
Potential EMS-induced STOP codon mutations in exon
(abbreviated as E) 1, 2, 3 and 4 of BnGTR2-A3 and C3

| | BnGTR2-A3 | | | BnGTR2-C3 | | |
|---|---|---|---|---|---|---|
| | Nucleotide | | | Nucleotide | | |
| | position in | | | position in | | |
| | SEQ ID NO: | Wildtype | → Potential | SEQ ID NO: | Wildtype | → Potential |
| | 29 | codon | stop codons | 35 | codon | stop codons |
| E1 | 40-42 | CAA | TAA | 40-42 | CAA | TAA |
| | 133-135 | TGG | TAG TAA TGA | 130-132 | TGG | TGA TAA TAG |
| E2 | 512-514 | CGA | TGA TAA | 508-510 | CGA | TAA TGA |
| E3 | 682-684 | CAG | TAA TAG | 674-676 | CAA | TAA |
| | 739-741 | CAG | TAG TAA | 731-733 | CAG | TAA TAG |
| | 826-828 | CAG | TAA TAG | 818-820 | CAG | TAA TAG |
| | 880-882 | TGG | TAG TGA TAA | 872-874 | TGG | TAG TAA TGA |
| | 907-909 | CAG | TAG TAA | 899-901 | CAG | TAG TAA |
| | 940-942 | CAG | TAA TAG | 932-934 | CAG | TAA TAG |
| | 955-957 | TGG | TAG TAA TGA | 947-949 | TGG | TAA TAG TGA |
| | 1123-1125 | CAG | TAG TAA | 1115-1117 | CAG | TAG TAA |
| | 1129-1131 | TGG | TGA TAA TAG | 1121-1123 | TGG | TAG TAA TGA |
| | 1189-1191 | CAA | TAA | | | |
| | 1192-1194 | CAA | TAA | 1184-1186 | CAA | TAA |
| E4 | 1464-1466 | TGG | TAA TAG TGA | 1528-1530 | TGG | TAA TAG TGA |
| | 1482-1484 | CAA | TAA | 1546-1548 | CAA | TAA |
| | 1485-1487 | CAA | TAA | 1549-1551 | CAG | TAG TAA |
| | 1527-1529 | TGG | TAA TGA TAG | 1591-1593 | TGG | TGA TAG TAA |
| | 1563-1565 | CAA | TAA | 1627-1629 | CAA | TAA |
| | 1566-1568 | CAA | TAA | 1630-1632 | CAA | TAA |
| | 1587-1589 | CAA | TAA | 1651-1653 | CAA | TAA |
| | 1596-1598 | CAG | TAG TAA | 1660-1662 | CAG | TAG TAA |
| | 1758-1760 | CAA | TAA | 1822-1824 | CAA | TAA |
| | 1848-1850 | CAA | TAA | | | |
| | 1872-1874 | CGG | TAA TAG TGA | 1936-1938 | CGG | TAA TAG TGA |
| | 1905-1907 | TGG | TAA TAG TGA | 1969-1971 | TGG | TAG TAA TGA |
| | 1917-1919 | CAG | TAG TAA | 1981-1983 | CAG | TAA TAG |
| | 1962-1964 | CAG | TAG TAA | 2026-2028 | CAG | TAA TAG |
| | 1983-1985 | CAG | TAA TAG | 2047-2049 | CAG | TAG TAA |
| | 2091-2093 | CAG | TAA TAG | 2155-2157 | CAG | TAG TAA |
| | 2112-2114 | TGG | TAA TAG TGA | 2176-2178 | TGG | TAA TAG TGA |
| | 2214-2216 | TGG | TAG TGA TAA | 2278-2280 | TGG | TAG TGA TAA |
| | 2283-2285 | CAA | TAA | 2347-2349 | CAA | TAA |

*The corresponding amino acid position in SEQ ID NO: 30 and 36 corresponds to the number of the amino acid encoded by the amino acid codon at the indicated nucleotide positions in SEQ ID NO: 29 and 35, respectively, in the sequence listing.

TABLE 3d
Potential EMS-induced STOP codon mutations in exon 1, 2, 3
and 4 of BjGTR2-A1 and B1

| | BjGTR2-A1 | | | BnGTR2-B1 | | |
|---|---|---|---|---|---|---|
| | Nucleotide | | | Nucleotide | | |
| | position in | | | position in | | |
| | SEQ ID NO: | Wildtype | → Potential | SEQ ID NO: | Wildtype | → Potential |
| | 119 | codon | stop codons | 125 | codon | stop codons |
| E1 | 662-664 | CAA | TAA | | | |
| | 755-757 | TGG | TAA TGA TAG | | | |
| E2 | 1294-1296 | CGA | TAA TGA | 177-179 | CGA | TGA TAA |
| E3 | 1523-1525 | CAG | TAG TAA | 353-355 | CAG | TAG TAA |
| | 1610-1612 | CAG | TAG TAA | 410-412 | CAG | TAG TAA |
| | 1640-1642 | CGA | TAA TGA | 497-499 | CAG | TAA TAG |
| | 1664-1666 | TGG | TAG TAA TGA | 551-553 | TGG | TAA TAG TGA |
| | 1691-1693 | CAG | TAA TAG | 578-580 | CAG | TAG TAA |
| | 1724-1726 | CAG | TAA TAG | 611-613 | CAG | TAA TAG |
| | 1739-1741 | TGG | TGA TAG TAA | 626-628 | TGG | TAA TAG TGA |
| | 1859-1861 | CAA | TAA | 746-748 | CAA | TAA |
| | 1913-1915 | TGG | TAA TAG TGA | 794-796 | CAG | TAG TAA |
| | 1976-1978 | CAG | TAA TAG | 800-802 | TGG | TAA TAG TGA |
| E4 | | | | 863-865 | CAG | TAA TAG |
| | 2153-2155 | TGG | TAG TAA TGA | 1036-1038 | TGG | TGA TAG TAA |
| | 2171-2173 | CAA | TAA | 1054-1056 | CAA | TAA |
| | | | | 1057-1059 | CAA | TAA |
| | 2174-2176 | CAA | TAA | 1099-1101 | TGG | TAA TGA TAG |
| | 2216-2218 | TGG | TGA TAG TAA | 1135-1137 | CAA | TAA |
| | 2252-2254 | CAA | TAA | 1138-1140 | CAG | TAA TAG |
| | 2255-2257 | CAA | TAA | 1159-1161 | CAA | TAA |
| | 2276-2278 | CAA | TAA | 1168-1170 | CAG | TAG TAA |
| | 2285-2287 | CAG | TAA TAG | 1330-1332 | CAG | TAA TAG |
| | 2447-2449 | CAG | TAA TAG | 1477-1479 | TGG | TGA TAA TAG |
| | 2513-2515 | CGG | TGA TAG TAA | 1489-1491 | CAA | TAA |
| | 2594-2596 | TGG | TAA TAG TGA | 1534-1536 | CAG | TAG TAA |
| | 2606-2608 | CAA | TAA | 1555-1557 | CAA | TAA |
| | 2651-2653 | CAG | TAG TAA | 1663-1665 | CAG | TAA TAG |
| | 2672-2674 | CAG | TAA TAG | 1684-1686 | TGG | TGA TAA TAG |
| | 2780-2782 | CAG | TAA TAG | 1786-1788 | TGG | TGA TAA TAG |
| | 2801-2803 | TGG | TAA TGA TAG | 1852-1854 | CAA | TAA |
| | 2903-2905 | TGG | TAA TGA TAG | 1855-1857 | CAA | TAA |
| | 2969-2971 | CAA | TAA | | | |
| | 2972-2974 | CAA | TAA | | | |

*The corresponding amino acid position in SEQ ID NO: 120 and 126 corresponds to the number of the amino acid encoded by the amino acid codon at the indicated nucleotide positions in SEQ ID NO: 119 and 125, respectively, in the sequence listing.

TABLE 3e
Potential EMS-induced STOP codon mutations in exon 1, 2, 3
and 4 of BjGTR2-A2 and B2

| | BjGTR2-A2 | | | BnGTR2-B2 | | |
|---|---|---|---|---|---|---|
| | Nucleotide | | | Nucleotide | | |
| | position in | | | position in | | |
| | SEQ ID NO: | Wildtype | → Potential | SEQ ID NO: | Wildtype | → Potential |
| | 121 | codon | stop codons | 127 | codon | stop codons |
| E1 | 1069-1071 | CAG | TAA TAG | 413-415 | CAG | TAA TAG |
| | 1135-1137 | CAG | TAG TAA | 467-469 | CAA | TAA |
| | 1162-1164 | TGG | TAA TAG TGA | 479-481 | CAG | TAA TAG |
| | | | | 506-508 | TGG | TAA TAG TGA |
| E2 | | | | | | |
| E3 | 1892-1894 | CAA | TAA | 1334-1336 | CAA | TAA |
| | 1949-1951 | CAG | TAG TAA | 1391-1393 | CAG | TAA TAG |
| | 2036-2038 | CAG | TAG TAA | 1478-1480 | CAG | TAA TAG |
| | 2090-2092 | TGG | TAA TAG TGA | 1532-1534 | TGG | TAA TAG TGA |
| | 2117-2119 | CAG | TAA TAG | 1559-1561 | CAG | TAG TAA |
| | 2150-2152 | CAG | TAA TAG | 1592-1594 | CAG | TAA TAG |
| | 2165-2167 | TGG | TAA TGA TAG | 1607-1609 | TGG | TAA TAG TGA |
| | 2285-2287 | CAA | TAA | 1727-1729 | CAA | TAA |
| | 2333-2335 | CAG | TAA TAG | 1775-1777 | CAG | TAG TAA |
| | 2339-2341 | TGG | TAG TAA TGA | 1781-1783 | TGG | TAA TGA TAG |
| | 2402-2404 | CAG | TAA TAG | 1844-1846 | CAG | TAG TAA |
| E4 | 2555-2557 | TGG | TAG TAA TGA | 2011-2013 | TGG | TGA TAG TAA |
| | 2573-2575 | CAA | TAA | 2029-2031 | CAA | TAA |
| | 2576-2578 | CAA | TAA | 2032-2034 | CAA | TAA |
| | 2618-2620 | TGG | TGA TAG TAA | 2074-2076 | TGG | TGA TAG TAA |
| | 2654-2656 | CAA | TAA | 2110-2112 | CAA | TAA |
| | 2657-2659 | CAA | TAA | 2113-2115 | CAG | TAA TAG |
| | 2678-2680 | CAA | TAA | 2134-2136 | CAA | TAA |
| | 2687-2689 | CAG | TAG TAA | 2143-2145 | CAG | TAA TAG |
| | 2849-2851 | CAA | TAA | 2305-2307 | CAG | TAG TAA |
| | 2915-2917 | CGG | TAG TGA TAA | 2371-2373 | CGG | TGA TAA TAG |
| | 2963-2965 | CGG | TGA TAA TAG | 2419-2421 | CGG | TAG TAA TGA |
| | 2996-2998 | TGG | TAA TGA TAG | 2452-2454 | TGG | TAG TGA TAA |
| | 3008-3010 | CAG | TAA TAG | 2464-2466 | CAG | TAA TAG |
| | 3053-3055 | CAG | TAA TAG | 2509-2511 | CAG | TAG TAA |
| | 3074-3076 | CAG | TAA TAGH | 2530-2532 | CAG | TAG TAA |
| | 3182-3184 | CAG | TAG TAA | 2638-2640 | CAG | TAG TAA |
| | 3203-3205 | TGG | TGA TAG TAA | 2659-2661 | TGG | TGA TAG TAA |
| | 3305-3307 | TGG | TGA TAA TAG | 2761-2763 | TGG | TAG TGA TAA |
| | 3371-3373 | CAA | TAA | 2827-2829 | CAA | TAA |
| | 3374-3376 | CAA | TAA | 2930-2832 | CAA | TAA |

*The corresponding amino acid position in SEQ ID NO: 122 and 128 corresponds to the number of the amino acid encoded by the amino acid codon at the indicated nucleotide positions in SEQ ID NO: 121 and 127, respectively, in the sequence listing.

TABLE 3f
Potential EMS-induced STOP codon mutations in exon 1, 2, 3
and 4 of BjGTR2-A3 and B3

| | BjGTR2-A3 | | | BnGTR2-B3 | | |
|---|---|---|---|---|---|---|
| | Nucleotide | | | Nucleotide | | |
| | position in | | | position in | | |
| | SEQ ID NO: | Wildtype | → Potential | SEQ ID NO: | Wildtype | → Potential |
| | 123 | codon | stop codons | 129 | codon | stop codons |
| E1 | 72-74 | CAA | TAA | 829-831 | CAA | TAA |
| | 138-140 | CAG | TAG TAA | 877-879 | CAG | TAA TAG |
| | 165-167 | TGG | TAG TAA TGA | 904-906 | TGG | TAA TAG TGA |
| E2 | 543-545 | CGA | TGA TAA | 1357-1359 | CGA | TAA TGA |
| E3 | 711-713 | CAA | TAA | 1518-1520 | CAA | TAA |
| | 768-770 | CAG | TAG TAA | 1575-1577 | CAG | TAA TAG |
| | 855-857 | CAG | TAA TAG | 1662-1664 | CAG | TAA TAG |
| | 909-911 | TGG | TAA TGA TAG | 1716-1718 | TGG | TAA TAG TGA |
| | 936-938 | CAG | TAG TAA | 1743-1745 | CAG | TAG TAA |
| | 969-971 | CAG | TAA TAG | 1776-1778 | CAG | TAG TAA |
| | 984-986 | TGG | TAG TGA TAA | 1791-1793 | TGG | TAA TAG TGA |
| | 1152-1154 | CAG | TAA TAG | 1959-1961 | CAG | TAG TAA |
| | 1158-1160 | TGG | TAG TAA TGA | 1965-1967 | TGG | TAA TGA TAG |
| | 1221-1223 | CAA | TAA | 2028-2030 | CAA | TAA |
| E4 | 1496-1498 | TGG | TAA TAG TGA | 2297-2299 | TGG | TGA TAA TAG |
| | 1514-1516 | CAA | TAA | 2315-2317 | CAA | TAA |
| | 1517-1519 | CAA | TAA | 2318-2320 | CAA | TAA |
| | 1559-1561 | TGG | TGA TAA TAG | 2396-2398 | CAA | TAA |
| | 1595-1597 | CAA | TAA | 2399-2401 | CAA | TAA |
| | 1598-1600 | CAA | TAA | 2420-2422 | CAA | TAA |
| | 1619-1621 | CAA | TAA | 2429-2431 | CAG | TAG TAA |
| | 1628-1630 | CAG | TAG TAA | 2591-2593 | CAA | TAA |
| | 1790-1792 | CAA | TAA | 2705-2707 | CGG | TGA TAA TAG |
| | 1904-1906 | CGG | TGA TAA TAG | 2738-2740 | TGG | TAA TAG TGA |
| | 1937-1939 | TGG | TAG TGA TAA | 2750-2752 | CAG | TAG TAA |
| | 1949-1951 | CAG | TAA TAG | 2795-2797 | CAG | TAG TAA |
| | 1994-1996 | CAG | TAA TAG | 2816-2818 | CAG | TAA TAG |
| | 2015-2017 | CAG | TAA TAG | 2924-2926 | CAG | TAG TAA |
| | 2123-2125 | CAG | TAA TAG | 2945-2947 | TGG | TAA TGA TAG |
| | 2144-2146 | TGG | TGA TAG TAA | 3048-3049 | TGG | TGA TAG TAA |
| | 2246-2248 | TGG | TAG TGA TAA | 3116-3118 | CAA | TAA |
| | 2315-2317 | CAA | TAA | | | |

*The corresponding amino acid position in SEQ ID NO: 124 and 130 corresponds to the number of the amino acid encoded by the amino acid codon at the indicated nucleotide positions in SEQ ID NO: 123 and 129, respectively, in the sequence listing.

Obviously, mutations are not limited to the ones shown in the above tables and it is understood that analogous STOP codon mutations may be present in gtr alleles other than those depicted in the sequence listing and referred to in the tables above.

A missense mutation in a GTR allele, as used herein, is any mutation (deletion, insertion or substitution) in a GTR allele whereby one or more codons are changed into the coding DNA and the corresponding mRNA sequence of the corresponding wild type GTR allele, resulting in the substitution of one or more amino acids in the wild type GTR protein for one or more other amino acids in the mutant GTR protein. In one embodiment, a mutant GTR allele comprising a missense mutation is a GTR allele wherein one or more of the conserved amino acids indicated above is/are substituted. In another embodiment, a mutant GTR allele comprising a missense mutation is a GTR allele encoding a GTR protein wherein an amino acid at a position corresponding to position 126, 145, 192 or 359 in SEQ ID NO: 66 is substituted, such as those indicated in Table 7 of Example 3 below.

A frameshift mutation in a GTR allele, as used herein, is a mutation (deletion, insertion, duplication, and the like) in a GTR allele that results in the nucleic acid sequence being translated in a different frame downstream of the mutation. As indicated above splice site mutations can result in frameshifts. Possible EMS-induced splice site mutations in Arabidopsis and Brassica GTR sequences provided herein are those which result in a mutation at the GU-donor site at the 5′ splice site or at the AG-acceptor site at the 3′ splice site indicated in the sequence listing, for example which result in a G/C to A/T transition in these sites.

Amino Acid Sequences According to the Invention

Provided are both wild type (functional) GTR amino acid sequences and mutant GTR amino acid sequences (comprising one or more mutations, preferably mutations which result in a significantly reduced or no GSL transport activity of the GTR protein) from Brassicaceae, particularly from Brassica species, especially from Brassica napus, but also from other Brassica crop species. For example, Brassica species comprising an A and/or a C or a B genome may encode different GTR-A, GTR-B or GTR-C amino acids. In addition, mutagenesis methods can be used to generate mutations in wild type GTR alleles, thereby generating mutant alleles which can encode further mutant GTR proteins. In one embodiment the wild type and/or mutant GTR amino acid sequences are provided within a Brassica plant (i.e. endogenously). However, isolated GTR amino acid sequences (e.g. isolated from the plant or made synthetically), as well as variants thereof and fragments of any of these are also provided herein.

“GTR amino acid sequence” or “GTR variant amino acid sequence” according to the invention are amino acid sequences having at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 55%, at least 56%, at least 57%, at least 58%, at least 59%, at least 60%, at least 61%, at least 62%, at least 63%, at least 64%, at least 65%, at least 70%, at least 75%, at least 76%, at least 77%, at least 78%, at least 79%, at least 80%, at least 85%, at least 90%, at least 95%, 98%, 99% or 100% sequence identity with SEQ ID NO: 2. These amino acid sequences may also be referred to as being “essentially similar” or “essentially identical” to the GTR sequences provided in the sequence listing.

Amino acid sequences of GTR1 to 6 proteins have been isolated from Arabidopsis, of GTRx-Ay proteins from B. rapa, B. juncea and from B. napus and of GTRx-Cy proteins from B. oleracea and from B. napus and of GTRx-By proteins from B. juncea as depicted in the sequence listing. The wild type GTR amino acid sequences are depicted, while mutant sequences of these sequences, and of sequences essentially similar to these, are described herein below and in the Examples, with reference to the wild type GTR amino acid sequences.

“GTR1, 2, 3, 4, 5 or 6 amino acid sequences” or “GTR1, 2, 3, 4, 5 or 6 variant amino acid sequences” according to the invention are amino acid sequences having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, 96%, 97%, 98%, 99% or 100% sequence identity with SEQ ID NO: 2, 4, 6, 8, 10 or 12, respectively. These amino acid sequences may also be referred to as being “essentially similar” or “essentially identical” to the GTR sequences provided in the sequence listing.

Thus, the invention provides both amino acid sequences of wild type, functional GTR1, 2, 3, 4, 5 or 6 proteins, including variants and fragments thereof (as defined further below), as well as mutant amino acid sequences of any of these, whereby the mutation in the amino acid sequence preferably results in a significant reduction in or a complete abolishment of the GSL transport activity of the GTR protein as compared to the GSL transport activity of the corresponding wild type GTR protein. A significant reduction in or complete abolishment of the GSL transport activity of the GTR protein refers herein to a reduction in or abolishment of the GSL transport activity of the GTR protein, such that the GSL content is modified in specific parts of a plant expressing the mutant GTR protein as compared to a plant expressing the corresponding wild type GTR protein.

Both endogenous and isolated amino acid sequences are provided herein. Also provided are fragments of the GTR amino acid sequences and GTR variant amino acid sequences defined above. A “fragment” of a GTR amino acid sequence or variant thereof (as defined) may be of various lengths, such as at least 10, 12, 15, 18, 20, 50, 100, 150, 175, 180 contiguous amino acids of the GTR sequence (or of the variant sequence).

Amino Acid Sequences of Functional GTR Proteins

The amino acid sequences depicted in the sequence listing encode wild type, functional GTR proteins from Arabidopsis, B. rapa, B. oleracea, B. juncea and B. napus. Thus, these sequences are endogenous to the plants from which they were isolated. Other Brassicaceae plants, including Brassica crop species, varieties, breeding lines or wild accessions, may be screened for other functional GTR proteins with the same amino acid sequences or variants thereof, as described above.

In addition, it is understood that GTR amino acid sequences and variants thereof (or fragments of any of these) may be identified in silico, by screening amino acid databases for essentially similar sequences. Fragments of amino acid molecules according to the invention are also provided. Fragments include amino acid sequences of conserved and functional domains as indicated above.

Amino Acid Sequences of Mutant GTR Proteins

Amino acid sequences comprising one or more amino acid deletions, insertions or substitutions relative to the wild type amino acid sequences are another embodiment of the invention, as are fragments of such mutant amino acid molecules. Such mutant amino acid sequences can be generated and/or identified using various known methods, as described above. Again, such amino acid molecules are provided both in endogenous form and in isolated form.

In one embodiment, the mutation(s) in the amino acid sequence result in a significantly reduced or completely abolished GSL transport activity of the GTR protein relative to the wild type protein. As described above, basically, any mutation which results in a protein comprising at least one amino acid insertion, deletion and/or substitution relative to the wild type protein can lead to significantly reduced or no GSL transport activity. It is, however, understood that mutations in certain parts of the protein are more likely to result in a reduced function of the mutant GTR protein, such as mutations leading to truncated proteins, whereby significant portions of the functional domains, such as transmembrane domains or substrate binding domains, are lacking or are being substituted. In one aspect, a mutant GTR protein comprising a missense mutation in a transmembrane domain is a GTR protein wherein an amino acid at a position corresponding to position 126 or to position 359 in SEQ ID NO: 66 is substituted, such as the mutant GTR protein indicated in Table 7 of Example 3 below.

Thus in one embodiment, mutant GTR proteins are provided comprising one or more deletion or insertion mutations, whereby the deletion(s) or insertion(s) result(s) in a mutant protein which has significantly reduced or no activity in vivo. Such mutant GTR proteins are GTR proteins wherein at least 1, at least 2, 3, 4, 5, 10, 20, 30, 50, 100, 100, 150, 175, 180 or more amino acids are deleted or inserted as compared to the wild type GTR protein, whereby the deletion(s) or insertion(s) result(s) in a mutant protein which has significantly reduced or no activity in vivo.

In another embodiment, mutant GTR proteins are provided which are truncated whereby the truncation results in a mutant protein that has significantly reduced or no activity in vivo. Such truncated GTR proteins are GTR proteins which lack functional domains in the C-terminal part of the corresponding wild type GTR protein and which maintain the N-terminal part of the corresponding wild type GTR protein. The more truncated the mutant protein is in comparison to the wild type protein, the more the truncation may result in a significantly reduced or no activity of the GTR protein.

In yet another embodiment, mutant GTR proteins are provided comprising one or more substitution mutations, whereby the substitution(s) result(s) in a mutant protein that has significantly reduced or no activity in vivo. Such mutant GTR proteins are GTR proteins whereby conserved amino acid residues which have a specific function, such as a function in substrate or proton binding, are substituted.

Also provided are variant GTR proteins which are changed in their phosphorylation/dephosphorylation status. Examples of such proteins are GTR1 or GTR2 proteins as herein described wherein the Serine or Threonine residues of phosphorylation sites are substituted for Aspartic acid (mimicking constitutive phosphorylation at that site) or for Alanine (mimicking constitutive dephosphorylation at that site) such as the GTR1 protein comprising the amino acid sequence of SEQ ID NO: 2 with the following substitutions:
a. S at position 22 for A
b. S at position 22 for D
c. T at position 105 for A
d. T at position 105 for D
e. S at position 605 for A
f. S at position 605 for D
   or the GTR1 protein comprising the amino acid sequence of SEQ ID NO: 142 with the following substitutions:
g. S at position 52 for A
h. S at position 52 for D
i. T at position 135 for A
j. T at position 135 for D
k. S at position 635 for A
l. S at position 635 for D
   or a GTR2 protein comprising the amino acid sequence of SEQ ID No: 4 with the following substitutions:
m. T at position 58 for A
n. T at position 58 for D
o. T at position 117 for A
p. T at position 117 for D
q. T at position 323 for A
r. T at position 323 for D
   Corresponding substitutions may be made in the GTR1 and GTR2 proteins from Brassica species, such as the ones described herein.

Methods According to the Invention

Mutant gtr alleles may be generated (for example induced by mutagenesis) and/or identified using a range of methods, which are conventional in the art, for example using PCR based methods to amplify part or all of the gtr genomic or cDNA.

Following mutagenesis, plants are grown from the treated seeds, or regenerated from the treated cells using known techniques. For instance, mutagenized seeds may be planted in accordance with conventional growing procedures and following self-pollination seed is formed on the plants. Alternatively, doubled haploid plantlets may be extracted from treated microspore or pollen cells to immediately form homozygous plants, for example as described by Coventry et al. (1988, Manual for Microspore Culture Technique for Brassica napus. Dep. Crop Sci. Techn. Bull. OAC Publication 0489. Univ. of Guelph, Guelph, Ontario, Canada). Additional seed which is formed as a result of such self-pollination in the present or a subsequent generation may be harvested and screened for the presence of mutant GTR alleles, using techniques which are conventional in the art, for example polymerase chain reaction (PCR) based techniques (amplification of the gtr alleles) or hybridization based techniques, e.g. Southern blot analysis, BAC library screening, and the like, and/or direct sequencing of gtr alleles. Several techniques are known to screen for specific mutant alleles, e.g., Deleteagene™ (Delete-a-gene; Li et al., 2001, Plant J 27: 235-242) uses polymerase chain reaction (PCR) assays to screen for deletion mutants generated by fast neutron mutagenesis, TILLING (targeted induced local lesions in genomes; McCallum et al., 2000, Nat Biotechnol 18:455-457) identifies EMS-induced point mutations, etc. To screen for the presence of point mutations (so called Single Nucleotide Polymorphisms or SNPs) in mutant GTR alleles, SNP detection methods conventional in the art can be used, for example oligoligation-based techniques, single base extension-based techniques or techniques based on differences in restriction sites, such as TILLING.

As described above, mutagenization (spontaneous as well as induced) of a specific wild-type GTR allele results in the presence of one or more deleted, inserted, or substituted nucleotides (hereinafter called “mutation region”) in the resulting mutant GTR allele. The mutant GTR allele can thus be characterized by the location and the configuration of the one or more deleted, inserted, or substituted nucleotides in the wild type GTR allele. The site in the wild type GTR allele where the one or more nucleotides have been inserted, deleted, or substituted, respectively, is herein also referred to as the “mutation region or sequence”. A “5′ or 3′ flanking region or sequence” as used herein refers to a DNA region or sequence in the mutant (or the corresponding wild type) GTR allele of at least 20 bp, preferably at least 50 bp, at least 750 bp, at least 1500 bp, and up to 5000 bp of DNA different from the DNA containing the one or more deleted, inserted, or substituted nucleotides, preferably DNA from the mutant (or the corresponding wild type) GTR allele which is located either immediately upstream of and contiguous with (5′ flanking region or sequence“) or immediately downstream of and contiguous with (3′ flanking region or sequence”) the mutation region in the mutant GTR allele (or in the corresponding wild type GTR allele). A “joining region” as used herein refers to a DNA region in the mutant (or the corresponding wild type) GTR allele where the mutation region and the 5′ or 3′ flanking region are linked to each other. A “sequence spanning the joining region between the mutation region and the 5′ or 3′ flanking region thus comprises a mutation sequence as well as the flanking sequence contiguous therewith.

The tools developed to identify a specific mutant GTR allele or the plant or plant material comprising a specific mutant GTR allele, or products which comprise plant material comprising a specific mutant GTR allele are based on the specific genomic characteristics of the specific mutant GTR allele as compared to the genomic characteristics of the corresponding wild type GTR allele, such as, a specific restriction map of the genomic region comprising the mutation region, molecular markers or the sequence of the flanking and/or mutation regions.

Once a specific mutant GTR allele has been sequenced, primers and probes can be developed which specifically recognize a sequence within the 5′ flanking, 3′ flanking and/or mutation regions of the mutant GTR allele in the nucleic acid (DNA or RNA) of a sample by way of a molecular biological technique. For instance a PCR method can be developed to identify the mutant GTR allele in biological samples (such as samples of plants, plant material or products comprising plant material). Such a PCR is based on at least two specific “primers”: one recognizing a sequence within the 5′ or 3′ flanking region of the mutant GTR allele and the other recognizing a sequence within the 3′ or 5′ flanking region of the mutant GTR allele, respectively; or one recognizing a sequence within the 5′ or 3′ flanking region of the mutant GTR allele and the other recognizing a sequence within the mutation region of the mutant GTR allele; or one recognizing a sequence within the 5′ or 3′ flanking region of the mutant GTR allele and the other recognizing a sequence spanning the joining region between the 3′ or 5′ flanking region and the mutation region of the specific mutant GTR allele (as described further below), respectively.

The primers preferably have a sequence of between 15 and 35 nucleotides which under optimized PCR conditions “specifically recognize” a sequence within the 5′ or 3′ flanking region, a sequence within the mutation region, or a sequence spanning the joining region between the 3′ or 5′ flanking and mutation regions of the specific mutant GTR allele, so that a specific fragment (“mutant GTR specific fragment” or discriminating amplicon) is amplified from a nucleic acid sample comprising the specific mutant GTR allele. This means that only the targeted mutant GTR allele, and no other sequence in the plant genome, is amplified under optimized PCR conditions.

PCR primers suitable for the invention may be the following:
oligonucleotides ranging in length from 17 nt to about 200 nt, comprising a nucleotide sequence of at least 17 consecutive nucleotides, preferably 20 consecutive nucleotides selected from the 5′ or 3′ flanking sequence of a specific mutant GTR allele or the complement thereof (i.e., for example, the sequence 5′ or 3′ flanking the one or more nucleotides deleted, inserted or substituted in the mutant GTR alleles of the invention, such as the sequence 5′ or 3′ flanking the non-sense, mis-sense or frameshift mutations described above or the sequence 5′ or 3′ flanking the STOP codon mutations indicated in the above Tables or the substitution mutations indicated above or the complement thereof) (primers recognizing 5′ flanking sequences); or
oligonucleotides ranging in length from 17 nt to about 200 nt, comprising a nucleotide sequence of at least 17 consecutive nucleotides, preferably 20 nucleotides selected from the sequence of the mutation region of a specific mutant GTR allele or the complement thereof (i.e., for example, the sequence of nucleotides inserted or substituted in the GTR genes of the invention or the complement thereof) (primers recognizing mutation sequences).

The primers may of course be longer than the mentioned 17 consecutive nucleotides, and may e.g. be 18, 19, 20, 21, 30, 35, 50, 75, 100, 150, 200 nt long or even longer. The primers may entirely consist of nucleotide sequence selected from the mentioned nucleotide sequences of flanking and mutation sequences. However, the nucleotide sequence of the primers at their 5′ end (i.e. outside of the 3′-located 17 consecutive nucleotides) is less critical. Thus, the 5′ sequence of the primers may consist of a nucleotide sequence selected from the flanking or mutation sequences, as appropriate, but may contain several (e.g. 1, 2, 5, 10) mismatches. The 5′ sequence of the primers may even entirely consist of a nucleotide sequence unrelated to the flanking or mutation sequences, such as e.g. a nucleotide sequence representing restriction enzyme recognition sites. Such unrelated sequences or flanking DNA sequences with mismatches should preferably be not longer than 100, more preferably not longer than 50 or even 25 nucleotides.

Moreover, suitable primers may comprise or consist of a nucleotide sequence spanning the joining region between flanking and mutation sequences (i.e., for example, the joining region between a sequence 5′ or 3′ flanking one or more nucleotides deleted, inserted or substituted in the mutant GTR alleles of the invention and the sequence of the one or more nucleotides inserted or substituted or the sequence 3′ or 5′, respectively, flanking the one or more nucleotides deleted, such as the joining region between a sequence 5′ or 3′ flanking non-sense, missense or frameshift mutations in the GTR genes of the invention described above and the sequence of the non-sense, missense or frameshift mutations, or the joining region between a sequence 5′ or 3′ flanking a potential STOP codon mutation as indicated in the above Tables or the substitution mutations indicated above and the sequence of the potential STOP codon mutation or the substitution mutations, respectively), provided the nucleotide sequence is not derived exclusively from either the mutation region or flanking regions.

It will also be immediately clear to the skilled artisan that properly selected PCR primer pairs should also not comprise sequences complementary to each other.

For the purpose of the invention, the “complement of a nucleotide sequence represented in SEQ ID No: X” is the nucleotide sequence which can be derived from the represented nucleotide sequence by replacing the nucleotides through their complementary nucleotide according to Chargaff's rules (A [symbol] T; G [symbol] C) and reading the sequence in the 5′ to 3′ direction, i.e. in opposite direction of the represented nucleotide sequence.

Examples of primers suitable to identify specific mutant GTR alleles are described in the Examples.

As used herein, “the nucleotide sequence of SEQ ID No. Z from position X to position Y” indicates the nucleotide sequence including both nucleotide endpoints.

Preferably, the amplified fragment has a length of between 50 and 1000 nucleotides, such as a length between 50 and 500 nucleotides, or a length between 100 and 350 nucleotides. The specific primers may have a sequence which is between 80 and 100% identical to a sequence within the 5′ or 3′ flanking region, to a sequence within the mutation region, or to a sequence spanning the joining region between the 3′ or 5′ flanking and mutation regions of the specific mutant GTR allele, provided the mismatches still allow specific identification of the specific mutant GTR allele with these primers under optimized PCR conditions. The range of allowable mismatches however, can easily be determined experimentally and are known to a person skilled in the art.

Detection and/or identification of a “mutant GTR specific fragment” can occur in various ways, e.g., via size estimation after gel or capillary electrophoresis or via fluorescence-based detection methods. The mutant GTR specific fragments may also be directly sequenced. Other sequence specific methods for detection of amplified DNA fragments are also known in the art.

Standard PCR protocols are described in the art, such as in ‘PCR Applications Manual” (Roche Molecular Biochemicals, 2nd Edition, 1999) and other references. The optimal conditions for the PCR, including the sequence of the specific primers, is specified in a “PCR identification protocol” for each specific mutant GTR allele. It is however understood that a number of parameters in the PCR identification protocol may need to be adjusted to specific laboratory conditions, and may be modified slightly to obtain similar results. For instance, use of a different method for preparation of DNA may require adjustment of, for instance, the amount of primers, polymerase, MgCl₂ concentration or annealing conditions used. Similarly, the selection of other primers may dictate other optimal conditions for the PCR identification protocol. These adjustments will however be apparent to a person skilled in the art, and are furthermore detailed in current PCR application manuals such as the one cited above.

Examples of PCR identification protocols to identify specific mutant GTR alleles are described in the Examples.

Alternatively, specific primers can be used to amplify a mutant GTR specific fragment that can be used as a “specific probe” for identifying a specific mutant GTR allele in biological samples. Contacting nucleic acid of a biological sample, with the probe, under conditions that allow hybridization of the probe with its corresponding fragment in the nucleic acid, results in the formation of a nucleic acid/probe hybrid. The formation of this hybrid can be detected (e.g. labeling of the nucleic acid or probe), whereby the formation of this hybrid indicates the presence of the specific mutant GTR allele. Such identification methods based on hybridization with a specific probe (either on a solid phase carrier or in solution) have been described in the art. The specific probe is preferably a sequence that, under optimized conditions, hybridizes specifically to a region within the 5′ or 3′ flanking region and/or within the mutation region of the specific mutant GTR allele (hereinafter referred to as “mutant GTR specific region”). Preferably, the specific probe comprises a sequence of between 10 and 1000 bp, 50 and 600 bp, between 100 to 500 bp, between 150 to 350 bp, which is at least 80%, preferably between 80 and 85%, more preferably between 85 and 90%, especially preferably between 90 and 95%, most preferably between 95% and 100% identical (or complementary) to the nucleotide sequence of a specific region. Preferably, the specific probe will comprise a sequence of about 13 to about 100 contiguous nucleotides identical (or complementary) to a specific region of the specific mutant GTR allele.

Specific probes suitable for the invention may be the following:
oligonucleotides ranging in length from 13 nt to about 1000 nt, comprising a nucleotide sequence of at least 13 consecutive nucleotides selected from the 5′ or 3′ flanking sequence of a specific mutant GTR allele or the complement thereof (i.e., for example, the sequence 5′ or 3′ flanking the one or more nucleotides deleted, inserted or substituted in the mutant GTR alleles of the invention, such as the sequence 5′ or 3′ flanking the non-sense, mis-sense or frameshift mutations described above or the sequence 5′ or 3′ flanking the potential STOP codon mutations indicated in the above Tables or the substitution mutations indicated above), or a sequence having at least 80% sequence identity therewith (probes recognizing 5′ flanking sequences); or
oligonucleotides ranging in length from 13 nt to about 1000 nt, comprising a nucleotide sequence of at least 13 consecutive nucleotides selected from the mutation sequence of a specific mutant GTR allele or the complement thereof (i.e., for example, the sequence of nucleotides inserted or substituted in the GTR genes of the invention, or the complement thereof), or a sequence having at least 80% sequence identity therewith (probes recognizing mutation sequences).

The probes may entirely consist of nucleotide sequence selected from the mentioned nucleotide sequences of flanking and mutation sequences. However, the nucleotide sequence of the probes at their 5′ or 3′ ends is less critical. Thus, the 5′ or 3′ sequences of the probes may consist of a nucleotide sequence selected from the flanking or mutation sequences, as appropriate, but may consist of a nucleotide sequence unrelated to the flanking or mutation sequences. Such unrelated sequences should preferably be not longer than 50, more preferably not longer than 25 or even not longer than 20 or 15 nucleotides.

Moreover, suitable probes may comprise or consist of a nucleotide sequence spanning the joining region between flanking and mutation sequences (i.e., for example, the joining region between a sequence 5′ or 3′ flanking one or more nucleotides deleted, inserted or substituted in the mutant GTR alleles of the invention and the sequence of the one or more nucleotides inserted or substituted or the sequence 3′ or 5′, respectively, flanking the one or more nucleotides deleted, such as the joining region between a sequence 5′ or 3′ flanking non-sense, mis-sense or frameshift mutations in the GTR genes of the invention described above and the sequence of the non-sense, mis-sense or frameshift mutations, or the joining region between a sequence 5′ or 3′ flanking a potential STOP codon mutation as indicated in the above Tables or the substitution mutations indicated above and the sequence of the potential STOP codon or substitution mutation, respectively), provided the mentioned nucleotide sequence is not derived exclusively from either the mutation region or flanking regions.

Examples of specific probes suitable to identify specific mutant GTR alleles are described in the Examples.

Detection and/or identification of a “mutant GTR specific region” hybridizing to a specific probe can occur in various ways, e.g., via size estimation after gel electrophoresis or via fluorescence-based detection methods. Other sequence specific methods for detection of a “mutant GTR specific region” hybridizing to a specific probe are also known in the art.

Alternatively, plants or plant parts comprising one or more mutant gtr alleles can be generated and identified using other methods, such as the “Delete-a-gene™” method which uses PCR to screen for deletion mutants generated by fast neutron mutagenesis (reviewed by Li and Zhang, 2002, Funct Integr Genomics 2:254-258), by the TILLING (Targeting Induced Local Lesions IN Genomes) method which identifies EMS-induced point mutations using denaturing high-performance liquid chromatography (DHPLC) to detect base pair changes by heteroduplex analysis (McCallum et al., 2000, Nat Biotech 18:455, and McCallum et al. 2000, Plant Physiol. 123, 439-442), etc. As mentioned, TILLING uses high-throughput screening for mutations (e.g. using Cel 1 cleavage of mutant-wildtype DNA heteroduplexes and detection using a sequencing gel system). Thus, the use of TILLING to identify plants or plant parts comprising one or more mutant gtr alleles and methods for generating and identifying such plants, plant organs, tissues and seeds is encompassed herein. Thus in one embodiment, the method according to the invention comprises the steps of mutagenizing plant seeds (e.g. EMS mutagenesis), pooling of plant individuals or DNA, PCR amplification of a region of interest, heteroduplex formation and high-throughput detection, identification of the mutant plant, sequencing of the mutant PCR product. It is understood that other mutagenesis and selection methods may equally be used to generate such mutant plants.

Instead of inducing mutations in GTR alleles, natural (spontaneous) mutant alleles may be identified by methods known in the art. For example, ECOTILLING may be used (Henikoff et al. 2004, Plant Physiology 135(2):630-6) to screen a plurality of plants or plant parts for the presence of natural mutant gtr alleles. As for the mutagenesis techniques above, preferably Brassica species are screened which comprise an A and/or a C genome, so that the identified gtr allele can subsequently be introduced into other Brassica species, such as Brassica napus, by crossing (inter- or intraspecific crosses) and selection. In ECOTILLING natural polymorphisms in breeding lines or related species are screened for by the TILLING methodology described above, in which individual or pools of plants are used for PCR amplification of the gtr target, heteroduplex formation and high-throughput analysis. This can be followed by selecting individual plants having a required mutation that can be used subsequently in a breeding program to incorporate the desired mutant allele.

The identified mutant alleles can then be sequenced and the sequence can be compared to the wild type allele to identify the mutation(s). Optionally functionality can be tested as indicated above. Using this approach a plurality of mutant gtr alleles (and Brassica plants comprising one or more of these) can be identified. The desired mutant alleles can then be combined with the desired wild type alleles by crossing and selection methods as described further below. Finally a single plant comprising the desired number of mutant gtr and the desired number of wild type GTR alleles is generated.

Oligonucleotides suitable as PCR primers or specific probes for detection of a specific mutant GTR allele can also be used to develop methods to determine the zygosity status of the specific mutant GTR allele.

To determine the zygosity status of a specific mutant GTR allele, a PCR-based assay can be developed to determine the presence of a mutant and/or corresponding wild type GTR specific allele:

To determine the zygosity status of a specific mutant GTR allele, two primers specifically recognizing the wild-type GTR allele can be designed in such a way that they are directed towards each other and have the mutation region located in between the primers. These primers may be primers specifically recognizing the 5′ and 3′ flanking sequences, respectively. This set of primers allows simultaneous diagnostic PCR amplification of the mutant, as well as of the corresponding wild type GTR allele.

Alternatively, to determine the zygosity status of a specific mutant GTR allele, two primers specifically recognizing the wild-type GTR allele can be designed in such a way that they are directed towards each other and that one of them specifically recognizes the mutation region. These primers may be primers specifically recognizing the sequence of the 5′ or 3′ flanking region and the mutation region of the wild type GTR allele, respectively. This set of primers, together with a third primer which specifically recognizes the sequence of the mutation region in the mutant GTR allele, allow simultaneous diagnostic PCR amplification of the mutant GTR gene, as well as of the wild type GTR gene.

Alternatively, to determine the zygosity status of a specific mutant GTR allele, two primers specifically recognizing the wild-type GTR allele can be designed in such a way that they are directed towards each other and that one of them specifically recognizes the joining region between the 5′ or 3′ flanking region and the mutation region. These primers may be primers specifically recognizing the 5′ or 3′ flanking sequence and the joining region between the mutation region and the 3′ or 5′ flanking region of the wild type GTR allele, respectively. This set of primers, together with a third primer which specifically recognizes the joining region between the mutation region and the 3′ or 5′ flanking region of the mutant GTR allele, respectively, allow simultaneous diagnostic PCR amplification of the mutant GTR gene, as well as of the wild type GTR gene.

Alternatively, the zygosity status of a specific mutant GTR allele can be determined by using alternative primer sets that specifically recognize mutant and wild type GTR alleles.

If the plant is homozygous for the mutant GTR gene or the corresponding wild type GTR gene, the diagnostic PCR assays described above will give rise to a single PCR product typical, preferably typical in length, for either the mutant or wild type GTR allele. If the plant is heterozygous for the mutant GTR allele, two specific PCR products will appear, reflecting both the amplification of the mutant and the wild type GTR allele.

Identification of the wild type and mutant GTR specific PCR products can occur e.g. by size estimation after gel or capillary electrophoresis (e.g. for mutant GTR alleles comprising a number of inserted or deleted nucleotides which results in a size difference between the fragments amplified from the wild type and the mutant GTR allele, such that said fragments can be visibly separated on a gel); by evaluating the presence or absence of the two different fragments after gel or capillary electrophoresis, whereby the diagnostic PCR amplification of the mutant GTR allele can, optionally, be performed separately from the diagnostic PCR amplification of the wild type GTR allele; by direct sequencing of the amplified fragments; or by fluorescence-based detection methods.

Examples of primers suitable to determine the zygosity of specific mutant GTR alleles are described in the Examples.

Alternatively, to determine the zygosity status of a specific mutant GTR allele, a hybridization-based assay can be developed to determine the presence of a mutant and/or corresponding wild type GTR specific allele:

To determine the zygosity status of a specific mutant GTR allele, two specific probes recognizing the wild-type GTR allele can be designed in such a way that each probe specifically recognizes a sequence within the GTR wild type allele and that the mutation region is located in between the sequences recognized by the probes. These probes may be probes specifically recognizing the 5′ and 3′ flanking sequences, respectively. The use of one or, preferably, both of these probes allows simultaneous diagnostic hybridization of the mutant, as well as of the corresponding wild type GTR allele.

Alternatively, to determine the zygosity status of a specific mutant GTR allele, two specific probes recognizing the wild-type GTR allele can be designed in such a way that one of them specifically recognizes a sequence within the GTR wild type allele upstream or downstream of the mutation region, preferably upstream of the mutation region, and that one of them specifically recognizes the mutation region. These probes may be probes specifically recognizing the sequence of the 5′ or 3′ flanking region, preferably the 5′ flanking region, and the mutation region of the wild type GTR allele, respectively. The use of one or, preferably, both of these probes, optionally, together with a third probe which specifically recognizes the sequence of the mutation region in the mutant GTR allele, allow diagnostic hybridization of the mutant and of the wild type GTR gene.

Alternatively, to determine the zygosity status of a specific mutant GTR allele, a specific probe recognizing the wild-type GTR allele can be designed in such a way that the probe specifically recognizes the joining region between the 5′ or 3′ flanking region, preferably the 5′ flanking region, and the mutation region of the wild type GTR allele. This probe, optionally, together with a second probe that specifically recognizes the joining region between the 5′ or 3′ flanking region, preferably the 5′ flanking region, and the mutation region of the mutant GTR allele, allows diagnostic hybridization of the mutant and of the wild type GTR gene.

Alternatively, the zygosity status of a specific mutant GTR allele can be determined by using alternative sets of probes that specifically recognize mutant and wild type GTR alleles.

If the plant is homozygous for the mutant GTR gene or the corresponding wild type GTR gene, the diagnostic hybridization assays described above will give rise to a single specific hybridization product, such as one or more hybridizing DNA (restriction) fragments, typical, preferably typical in length, for either the mutant or wild type GTR allele. If the plant is heterozygous for the mutant GTR allele, two specific hybridization products will appear, reflecting both the hybridization of the mutant and the wild type GTR allele.

Identification of the wild type and mutant GTR specific hybridization products can occur e.g. by size estimation after gel or capillary electrophoresis (e.g. for mutant GTR alleles comprising a number of inserted or deleted nucleotides which results in a size difference between the hybridizing DNA (restriction) fragments from the wild type and the mutant GTR allele, such that said fragments can be visibly separated on a gel); by evaluating the presence or absence of the two different specific hybridization products after gel or capillary electrophoresis, whereby the diagnostic hybridization of the mutant GTR allele can, optionally, be performed separately from the diagnostic hybridization of the wild type GTR allele; by direct sequencing of the hybridizing DNA (restriction) fragments; or by fluorescence-based detection methods.

Examples of probes suitable to determine the zygosity of specific mutant GTR alleles are described in the Examples.

Furthermore, detection methods specific for a specific mutant GTR allele that differ from PCR- or hybridization-based amplification methods can also be developed using the specific mutant GTR allele specific sequence information provided herein. Such alternative detection methods include linear signal amplification detection methods based on invasive cleavage of particular nucleic acid structures, also known as Invader™ technology, (as described e.g. in U.S. Pat. No. 5,985,557 “Invasive Cleavage of Nucleic Acids”, U.S. Pat. No. 6,001,567 “Detection of Nucleic Acid sequences by Invader Directed Cleavage, incorporated herein by reference), RT-PCR-based detection methods, such as Taqman, or other detection methods, such as SNPlex. Briefly, in the Invader™ technology, the target mutation sequence may e.g. be hybridized with a labeled first nucleic acid oligonucleotide comprising the nucleotide sequence of the mutation sequence or a sequence spanning the joining region between the 5′ flanking region and the mutation region and with a second nucleic acid oligonucleotide comprising the 3′ flanking sequence immediately downstream and adjacent to the mutation sequence, wherein the first and second oligonucleotide overlap by at least one nucleotide. The duplex or triplex structure that is produced by this hybridization allows selective probe cleavage with an enzyme (Cleavase®) leaving the target sequence intact. The cleaved labeled probe is subsequently detected, potentially via an intermediate step resulting in further signal amplification.

A “kit”, as used herein, refers to a set of reagents for the purpose of performing the method of the invention, more particularly, the identification of a specific mutant GTR allele in biological samples or the determination of the zygosity status of plant material comprising a specific mutant GTR allele. More particularly, a preferred embodiment of the kit of the invention comprises at least two specific primers, as described above, for identification of a specific mutant GTR allele, or at least two or three specific primers for the determination of the zygosity status. Optionally, the kit can further comprise any other reagent described herein in the PCR identification protocol. Alternatively, according to another embodiment of this invention, the kit can comprise at least one specific probe, which specifically hybridizes with nucleic acid of biological samples to identify the presence of a specific mutant GTR allele therein, as described above, for identification of a specific mutant GTR allele, or at least two or three specific probes for the determination of the zygosity status. Optionally, the kit can further comprise any other reagent (such as but not limited to hybridizing buffer, label) for identification of a specific mutant GTR allele in biological samples, using the specific probe.

The kit of the invention can be used, and its components can be specifically adjusted, for purposes of quality control (e.g., purity of seed lots), detection of the presence or absence of a specific mutant GTR allele in plant material or material comprising or derived from plant material, such as but not limited to food or feed products.

The term “primer” as used herein encompasses any nucleic acid that is capable of priming the synthesis of a nascent nucleic acid in a template-dependent process, such as PCR. Typically, primers are oligonucleotides from 10 to 30 nucleotides, but longer sequences can be employed. Primers may be provided in double-stranded form, though the single-stranded form is preferred. Probes can be used as primers, but are designed to bind to the target DNA or RNA and need not be used in an amplification process.

The term “recognizing” as used herein when referring to specific primers, refers to the fact that the specific primers specifically hybridize to a nucleic acid sequence in a specific mutant GTR allele under the conditions set forth in the method (such as the conditions of the PCR identification protocol), whereby the specificity is determined by the presence of positive and negative controls.

The term “hybridizing”, as used herein when referring to specific probes, refers to the fact that the probe binds to a specific region in the nucleic acid sequence of a specific mutant GTR allele under standard stringency conditions. Standard stringency conditions as used herein refers to the conditions for hybridization described herein or to the conventional hybridizing conditions as described by Sambrook et al., 1989 (Molecular Cloning: A Laboratory Manual, Second Edition, Cold Spring Harbour Laboratory Press, NY) which for instance can comprise the following steps: 1) immobilizing plant genomic DNA fragments or BAC library DNA on a filter, 2) prehybridizing the filter for 1 to 2 hours at 65° C. in 6×SSC, 5×Denhardt's reagent, 0.5% SDS and 20 μg/ml denaturated carrier DNA, 3) adding the hybridization probe which has been labeled, 4) incubating for 16 to 24 hours, 5) washing the filter once for 30 min. at 68° C. in 6×SSC, 0.1% SDS, 6) washing the filter three times (two times for 30 min. in 30 ml and once for 10 min in 500 ml) at 68° C. in 2×SSC, 0.1% SDS, and 7) exposing the filter for 4 to 48 hours to X-ray film at −70° C.

As used in herein, a “biological sample” is a sample of a plant, plant material or product comprising plant material. The term “plant” is intended to encompass plant tissues, at any stage of maturity, as well as any cells, tissues, or organs taken from or derived from any such plant, including without limitation, any seeds, leaves, stems, flowers, roots, single cells, gametes, cell cultures, tissue cultures or protoplasts. “Plant material”, as used herein refers to material that is obtained or derived from a plant. Products comprising plant material relate to food, feed or other products that are produced using plant material or can be contaminated by plant material. It is understood that, in the context of the present invention, such biological samples are tested for the presence of nucleic acids specific for a specific mutant GTR allele, implying the presence of nucleic acids in the samples. Thus the methods referred to herein for identifying a specific mutant GTR allele in biological samples, relate to the identification in biological samples of nucleic acids that comprise the specific mutant GTR allele.

The present invention also relates to the combination of specific GTR alleles in one plant, to the transfer of one or more specific mutant GTR allele(s) from one plant to another plant, to the plants comprising one or more specific mutant GTR allele(s), the progeny obtained from these plants and to plant cells, plant parts, and plant seeds derived from these plants.

Thus, in one embodiment of the invention a method for combining two or more selected mutant GTR alleles in one plant is provided comprising the steps of:
(a) generating and/or identifying two or more plants each comprising one or more selected mutant GTR alleles, as described above,
(b) crossing a first plant comprising one or more selected mutant GTR alleles with a second plant comprising one or more other selected mutant GTR alleles, collecting F1 seeds from the cross, and, optionally, identifying an F1 plant comprising one or more selected mutant GTR alleles from the first plant with one or more selected mutant GTR alleles from the second plant, as described above,
(c) optionally, repeating step (b) until an F1 plant comprising all selected mutant GTR alleles is obtained,
(d) optionally,
   identifying an F1 plant, which is homozygous or heterozygous for a selected mutant GTR allele by determining the zygosity status of the mutant GTR alleles, as described above, or
   generating plants which are homozygous for one or more of the selected mutant GTR alleles by performing one of the following steps:
      extracting doubled haploid plants from treated microspore or pollen cells of F1 plants comprising the one or more selected mutant GTR alleles, as described above,
      selfing the F1 plants comprising the one or more selected mutant GTR allele(s) for one or more generations (y), collecting F1 Sy seeds from the selfings, and identifying F1 Sy plants, which are homozygous for the one or more mutant GTR allele, as described above.

In another embodiment of the invention a method for transferring one or more mutant GTR alleles from one plant to another plant is provided comprising the steps of:
(a) generating and/or identifying a first plant comprising one or more selected mutant GTR alleles, as described above, or generating the first plant by combining the one or more selected mutant GTR alleles in one plant, as described above (wherein the first plant is homozygous or heterozygous for the one or more mutant GTR alleles),
(b) crossing the first plant comprising the one or more mutant GTR alleles with a second plant not comprising the one or more mutant GTR alleles, collecting F1 seeds from the cross (wherein the seeds are heterozygous for a mutant GTR allele if the first plant was homozygous for that mutant GTR allele, and wherein half of the seeds are heterozygous and half of the seeds are azygous for, i.e. do not comprise, a mutant GTR allele if the first plant was heterozygous for that mutant GTR allele), and, optionally, identifying F1 plants comprising one or more selected mutant GTR alleles, as described above,
(c) backcrossing F1 plants comprising one or more selected mutant GTR alleles with the second plant not comprising the one or more selected mutant GTR alleles for one or more generations (x), collecting BCx seeds from the crosses, and identifying in every generation BCx plants comprising the one or more selected mutant GTR alleles, as described above,
(d) optionally, generating BCx plants which are homozygous for the one or more selected mutant GTR alleles by performing one of the following steps:
   extracting doubled haploid plants from treated microspore or pollen cells of BCx plants comprising the one or more desired mutant GTR allele(s), as described above,
   selfing the BCx plants comprising the one or more desired mutant GTR allele(s) for one or more generations (y), collecting BCx Sy seeds from the selfings, and identifying BCx Sy plants, which are homozygous for the one or more desired mutant GTR allele, as described above.

In one aspect of the invention, the first and the second plant are Brassicaceae plants, particularly Brassica plants, especially Brassica napus plants or plants from another Brassica crop species, such as Brassica rapa, B. juncea or Brassica oleracea. In another aspect of the invention, the first plant is a Brassicaceae plant, particularly a Brassica plant, especially a Brassica napus plant or a plant from another Brassica crop species, and the second plant is a plant from a Brassicaceae breeding line, particularly from a Brassica breeding line, especially from a Brassica napus breeding line or from a breeding line from another Brassica crop species. “Breeding line”, as used herein, is a preferably homozygous plant line distinguishable from other plant lines by a preferred genotype and/or phenotype that is used to produce hybrid offspring.

The inventors further found that seeds of Arabidopsis plants knocked out in either GTR1 or GTR2 transporters had no significant reduction and about 50% reduction in total aliphatic GSLs concentrations, respectively, compared to wildtype plants, and that seeds of Arabidopsis plants knocked out in both GTR1 and GTR2 transporters had a zero GSL seed phenotype. In addition, GSLs levels were decreased in inflorescences and in root tissue of gtr knockout plants compared to GSLs levels in these tissues in wildtype plants. Surprisingly, GSLs levels in senescent leaves from gtr knockout plants were high whereas, in wildtype plants, leaves become depleted in GSLs upon aging. In addition, GSLs levels in silique walls of gtr knockout plants increased compared to wildtype plants. Similar observations were made in B. rapa.

Thus, the inventors found that Brassicaceae plants, wherein the GTR activity is reduced, in particular the GTR2 activity or the GTR1 and GTR2 activity, have a decreased to undetectable GSL content in seed, inflorescence tissue and root tissue, while the levels of GSLs in green tissues (such as rosette leaves, cauline leaves, silique walls) remain high. The observations indicate that the GTR proteins of the present invention, in particular the GTR1 and GRT2 proteins, are essential components of a transport pathway involved in the transport of GSLs from green tissues, such as rosette leaves, cauline leaves and silique walls (so-called “source” tissues), into seeds, flowers and, at a certain period in the plant's life cycle, into the roots (so-called “sink” tissues).

In one embodiment, the invention provides a method to modify GSL transport in eukaryotic cells or organisms, such as Xenopus oocytes and plants, comprising modifying the functional GTR activity in said eukaryotic cells or organisms.

In another embodiment, the invention provides a method to modify the GSL content in plants and plant parts comprising modifying the functional GTR activity in said plant or plant parts.

Modification of the GSL content in plants and plant parts enables modifications to be made, for example, to meal quality of oilseeds crucifers, cancer preventive activity and flavour of horticultural crucifers, and/or resistance to herbivores and pathogens and biofumigative activity.

In one aspect, the GSL content is decreased in plant seed by reducing the functional GTR activity.

As used herein, “seed” comprises embryo, endosperm and/or seed coat.

In another aspect, the GSL content is increased or maintained in green plant tissue by reducing the functional GTR activity.

As used herein, “green plant tissue” refers to leaves, rosette leaves, cauline leaves and silique walls.

In yet another aspect, the GSL content is decreased in plant seed and increased in green plant tissue by reducing the functional GTR activity.

In one embodiment of the invention, the plant is a plant from the Brassicales or Capparales order having a high content of GSLs. Non-limiting examples of such plants are: plants of the Akaniaceae family, the Bataceae family, the Brassicaceae or Cruciferae family, the Capparaceae family, the Caricaceae family, the Gyrostemonaceae family, the Koeberliniaceae family, the Limnanthaceae family, the Moringaceae family, the Pentadiplandraceae family, the Resedaceae family, the Salvadoraceae family, the Setchellanthaceae family, the Tovariaceae family and the Tropaeolaceae family.

In a specific embodiment of the invention, the plant belongs to the Brassicaceae family. In an even more specific embodiment of the invention, the plant is a Brassica napus plant (such as rapeseed, canola and rutabaga), a Brassica rapa plant (such as Chinese cabbage and turnip), a Brassica oleracea plant (such as kale, cabbage, broccoli, cauliflower, Brussels sprouts and kohlrabi), a Brassica carinata plant (Abyssinian mustard), a Brassica juncea plant (Indian mustard) or a Brassica nigra plant (black mustard).

The most important crops for modification of seed meal quality by, e.g., reducing the GSL content in seed, are oilseed forms of Brassica spp. (e.g. B. napus, B. rapa (syn B. campestris), B. juncea and B. carinata).

For enhancement of flavour and cancer preventive properties by, e.g., increasing the GSL content in green plant tissues, the most important species are B. oleracea (including e.g. broccoli and cauliflower), horticultural forms of B. napus (e.g. swedes [=rutabaga, spp. napobrassica], oil seed rape) and B. rapa (including both turnips and Chinese cabbage [=pakchois]), cruciferous salads (including e.g. Eruca sativa and Diplotaxis tenuifolia) and horticultural forms of Raphanus (e.g. radish (Raphanus sativa)).

GSL content can be modified by reducing functional GTR activity. As used herein, “functional GTR activity” in a plant or plant part refers to the GTR activity as present in said plant or plant part. Functional GTR activity is the result of GTR gene expression level and GTR activity. Accordingly, the functional GTR activity in a plant or plant part can be reduced by down-regulating GTR gene expression level or by down-regulating GTR activity, or both and, according to the invention, the modification of GSL content of a plant, plant tissue, plant organ, plant part, or plant cell can be achieved by down-regulation of GTR gene expression level, by down-regulation of GTR activity, or both.

Conveniently, GTR gene expression level or GTR activity is controlled genetically by introduction of chimeric genes altering the GTR gene expression level and/or by introduction of chimeric genes altering the GTR activity and/or by alteration of the endogenous GTR-encoding genes.

In accordance with the invention, it is preferred that in order to modify GSL content, the functional GTR activity is reduced significantly. Preferably, the functional GTR activity in the target cells should be decreased about 75%, preferably about 80%, particularly about 90%, more particularly about 95%, more preferably about 100% of the normal level and/or activity in the target cells. Methods to determine the content of a specific protein such as the GTR proteins are well known to the person skilled in the art and include, but are not limited to (histochemical) quantification of such proteins using specific antibodies. A method to quantify GTR activity is described in the Examples below.

Thus in one embodiment of the invention, a method for modifying the GSL content of a plant or plant part comprises the step of down-regulating GTR gene expression. In another embodiment of the invention, a method for modifying the GSL content of a plant, plant tissue, plant organ, plant part, or plant cell comprises down-regulating GTR activity.

In an embodiment of the invention, GTR gene expression is down-regulated by introducing a chimeric DNA construct in the plant or plant part comprising the following operably linked DNA regions:
a) a plant-expressible promoter which functions in the plant or plant part;
b) a DNA region which when transcribed yields a GTR-inhibitory RNA molecule capable of down-regulating GTR gene expression; and
c) a DNA region involved in transcription termination and polyadenylation, functional in plant cells.

The transcribed DNA region encodes a biologically active RNA which decreases the levels of GTR mRNAs available for translation. This can be achieved through well established techniques including co-suppression (sense RNA suppression), antisense RNA, double-stranded RNA (dsRNA), or microRNA (miRNA).

In one embodiment, GTR gene expression may be down-regulated by introducing a chimeric DNA construct which yields a sense RNA molecule capable of down-regulating GTR gene expression by co-suppression. The transcribed DNA region will yield upon transcription a so-called sense RNA molecule capable of reducing the expression of a GTR gene in the target plant or plant cell in a transcriptional or post-transcriptional manner. The transcribed DNA region (and resulting RNA molecule) comprises at least 20 consecutive nucleotides having at least 95% sequence identity to the nucleotide sequence of a GTR-encoding gene present in the plant cell or plant.

In another embodiment, GTR gene expression may be down-regulated by introducing a chimeric DNA construct which yields an antisense RNA molecule capable of down-regulating GTR gene expression. The transcribed DNA region will yield upon transcription a so-called antisense RNA molecule capable of reducing the expression of a GTR gene in the target plant or plant cell in a transcriptional or post-transcriptional manner. The transcribed DNA region (and resulting RNA molecule) comprises at least 20 consecutive nucleotides having at least 95% sequence identity to the complement of the nucleotide sequence of a GTR-encoding gene present in the plant cell or plant.

However, the minimum nucleotide sequence of the antisense or sense RNA region of about 20 nt of the GTR-encoding region may be comprised within a larger RNA molecule, varying in size from 20 nt to a length equal to the size of the target gene. The mentioned antisense or sense nucleotide regions may thus be about from about 21 nt to about 5000 nt long, such as 21 nt, 40 nt, 50 nt, 100 nt, 200 nt, 300 nt, 500 nt, 1000 nt, 2000 nt or even about 5000 nt or larger in length. Moreover, it is not required for the purpose of the invention that the nucleotide sequence of the used inhibitory GTR RNA molecule or the encoding region of the transgene, is completely identical or complementary to the endogenous GTR gene the expression of which is targeted to be reduced in the plant cell. The longer the sequence, the less stringent the requirement for the overall sequence identity is. Thus, the sense or antisense regions may have an overall sequence identity of about 40% or 50% or 60% or 70% or 80% or 90% or 100% to the nucleotide sequence of the endogenous GTR gene or the complement thereof. However, as mentioned, antisense or sense regions should comprise a nucleotide sequence of 20 consecutive nucleotides having about 95 to about 100% sequence identity to the nucleotide sequence of the endogenous GTR gene. The stretch of about 95 to about 100% sequence identity may be about 50, 75 or 100 nt.

The efficiency of the above mentioned chimeric genes for antisense RNA or sense RNA-mediated gene expression level down-regulation may be further enhanced by inclusion of DNA elements which result in the expression of aberrant, non-polyadenylated GTR inhibitory RNA molecules. One such DNA element suitable for that purpose is a DNA region encoding a self-splicing ribozyme, as described in WO 00/01133. The efficiency may also be enhanced by providing the generated RNA molecules with nuclear localization or retention signals as described in WO 03/076619.

In yet another embodiment, GTR gene expression may be down-regulated by introducing a chimeric DNA construct which yields a double-stranded RNA molecule capable of down-regulating GTR gene expression. Upon transcription of the DNA region the RNA is able to form dsRNA molecule through conventional base paring between a sense and antisense region, whereby the sense and antisense region are nucleotide sequences as hereinbefore described. dsRNA-encoding GTR expression-reducing chimeric genes according to the invention may further comprise an intron, such as a heterologous intron, located e.g. in the spacer sequence between the sense and antisense RNA regions in accordance with the disclosure of WO 99/53050 (incorporated herein by reference). To achieve the construction of such a transgene, use can be made of the vectors described in WO 02/059294 A1.

In still another embodiment, GTR gene expression is down-regulated by introducing a chimeric DNA construct which yields a pre-miRNA RNA molecule which is processed into a miRNA capable of guiding the cleavage of GTR mRNA. miRNAs are small endogenous RNAs that regulate gene expression in plants, but also in other eukaryotes. In plants, these about 21 nucleotide long RNAs are processed from the stem-loop regions of long endogenous pre-miRNAs by the cleavage activity of DICERLIKE1 (DCL1). Plant miRNAs are highly complementary to conserved target mRNAs, and guide the cleavage of their targets. miRNAs appear to be key components in regulating the gene expression of complex networks of pathways involved inter alia in development.

As used herein, a “miRNA” is an RNA molecule of about 20 to 22 nucleotides in length which can be loaded into a RISC complex and direct the cleavage of a target RNA molecule, wherein the target RNA molecule comprises a nucleotide sequence essentially complementary to the nucleotide sequence of the miRNA molecule whereby one or more of the following mismatches may occur:
A mismatch between the nucleotide at the 5′ end of said miRNA and the corresponding nucleotide sequence in the target RNA molecule;
A mismatch between any one of the nucleotides in position 1 to position 9 of said miRNA and the corresponding nucleotide sequence in the target RNA molecule;
Three mismatches between any one of the nucleotides in position 12 to position 21 of said miRNA and the corresponding nucleotide sequence in the target RNA molecule provided that there are no more than two consecutive mismatches.
   No mismatch is allowed at positions 10 and 11 of the miRNA (all miRNA positions are indicated starting from the 5′ end of the miRNA molecule).

As used herein, a “pre-miRNA” molecule is an RNA molecule of about 100 to about 200 nucleotides, preferably about 100 to about 130 nucleotides which can adopt a secondary structure comprising a dsRNA stem and a single stranded RNA loop and further comprising the nucleotide sequence of the miRNA and its complement sequence of the miRNA* in the double-stranded RNA stem. Preferably, the miRNA and its complement are located about 10 to about 20 nucleotides from the free ends of the miRNA dsRNA stem. The length and sequence of the single stranded loop region are not critical and may vary considerably, e.g. between 30 and 50 nt in length. Preferably, the difference in free energy between unpaired and paired RNA structure is between −20 and −60 kcal/mole, particularly around −40 kcal/mole. The complementarity between the miRNA and the miRNA* do not need to be perfect and about 1 to 3 bulges of unpaired nucleotides can be tolerated. The secondary structure adopted by an RNA molecule can be predicted by computer algorithms conventional in the art such as mFold, UNAFold and RNAFold. The particular strand of the dsRNA stem from the pre-miRNA which is released by DCL activity and loaded onto the RISC complex is determined by the degree of complementarity at the 5′ end, whereby the strand which at its 5′ end is the least involved in hydrogen bounding between the nucleotides of the different strands of the cleaved dsRNA stem is loaded onto the RISC complex and will determine the sequence specificity of the target RNA molecule degradation. However, if empirically the miRNA molecule from a particular synthetic pre-miRNA molecule is not functional because the “wrong” strand is loaded on the RISC complex, it will be immediately evident that this problem can be solved by exchanging the position of the miRNA molecule and its complement on the respective strands of the dsRNA stem of the pre-miRNA molecule. As is known in the art, binding between A and U involving two hydrogen bounds, or G and U involving two hydrogen bounds is less strong that between G and C involving three hydrogen bounds.

miRNA molecules may be comprised within their naturally occurring pre-miRNA molecules but they can also be introduced into existing pre-miRNA molecule scaffolds by exchanging the nucleotide sequence of the miRNA molecule normally processed from such existing pre-miRNA molecule for the nucleotide sequence of another miRNA of interest. The scaffold of the pre-miRNA can also be completely synthetic. Likewise, synthetic miRNA molecules may be comprised within, and processed from, existing pre-miRNA molecule scaffolds or synthetic pre-miRNA scaffolds.

In another embodiment of the invention, GTR protein activity may be down-regulated by introducing a chimeric DNA construct in the plant, plant tissue, plant organ, plant part, or plant cell, comprising the following operably linked DNA regions:
a) a promoter, operative in the plant, plant tissue, plant organ, plant part, or plant cell;
b) a DNA region which when transcribed yields a GTR-inhibitory RNA molecule capable of down-regulating GTR activity; and
c) a DNA region involved in transcription termination and polyadenylation.

In one aspect, the GTR-inhibitory RNA molecule capable of down-regulating endogenous GTR protein activity is an RNA molecule which can be translated into a biologically active protein capable of decreasing the levels of GTR activity. This can be achieved, e.g., inactivating antibodies to GTR proteins. “Inactivating antibodies to GTR proteins” are antibodies or parts thereof which specifically bind at least to some epitopes of GTR proteins, such as the substrate/proton binding domain or the conserved domains described above, or which trap the transport protein in a conformation that does not allow transport (as described e.g. in JBC 274(22): 15420-15426, 1999) and which inhibit the activity of the target protein.

The chimeric DNA construct used to reduce the functional GTR activity by down-regulation of GTR gene expression level and/or by down-regulation of GTR protein activity can be stably inserted in a conventional manner into the nuclear genome of a single plant cell, and the so-transformed plant cell can be used in a conventional manner to produce a transformed plant with modified GSL content. In this regard, a T-DNA vector, containing the chimeric DNA construct used to reduce the functional GTR activity, in Agrobacterium tumefaciens can be used to transform the plant cell, and thereafter, a transformed plant can be regenerated from the transformed plant cell using the procedures described, for example, in EP0116718, EP0270822, WO84/02913 and published European Patent application EP 0 242 246 and in Gould et al. (1991). The construction of a T-DNA vector for Agrobacterium mediated plant transformation is well known in the art. The T-DNA vector may be either a binary vector as described in EP0120561 and EP0120515 or a co-integrate vector which can integrate into the Agrobacterium Ti-plasmid by homologous recombination, as described in EP0116718. Preferred T-DNA vectors each contain a promoter operably linked to the transcribed DNA region between T-DNA border sequences, or at least located to the left of the right border sequence. Border sequences are described in Gielen et al. (1984). Introduction of the T-DNA vector into Agrobacterium can be carried out using known methods, such as electroporation or triparental mating. Of course, other types of vectors can be used to transform the plant cell, using procedures such as direct gene transfer (as described, for example in EP0223247), pollen mediated transformation (as described, for example in EP0270356 and WO85/01856), protoplast transformation as, for example, described in U.S. Pat. No. 4,684,611, plant RNA virus-mediated transformation (as described, for example in EP0067553 and U.S. Pat. No. 4,407,956), liposome-mediated transformation (as described, for example in U.S. Pat. No. 4,536,475), and other methods The resulting transformed plant can be used in a conventional plant breeding scheme to produce more transformed plants with modifying total GSL content.

In another embodiment of the invention, the functional activity of GTR may be reduced by modification of the nucleotide sequence of the endogenous GTR genes. In a preferred embodiment, the GTR gene expression-regulating sequences are altered so that the GTR gene expression levels are down-regulated.

Methods to achieve such a modification of endogenous GTR genes include homologous recombination to exchange the endogenous GTR genes for mutant GTR genes e.g. by the methods described in U.S. Pat. No. 5,527,695. In a preferred embodiment such site-directed modification of the nucleotide sequence of the endogenous GTR genes is achieved by introduction of chimeric DNA/RNA oligonucleotides as described in WO 96/22364 or U.S. Pat. No. 5,565,350.

Methods to achieve such a modification of endogenous GTR genes also include mutagenesis. It will be immediately clear to the skilled artisan, that mutant plant cells and plant lines, wherein the functional GTR activity is reduced may be used to the same effect as the transgenic plant cells and plant lines described herein. Mutants in GTR gene of a plant cell or plant may be easily identified using screening methods known in the art, whereby chemical mutagenesis, such as e.g., EMS mutagenesis, is combined with sensitive detection methods (such as e.g., denaturing HPLC). An example of such a technique is the so-called “Targeted Induced Local Lesions in Genomes” method as described in McCallum et al, Plant Physiology 123 439-442 or WO 01/75167. However, other methods to detect mutations in particular genome regions or even alleles, are also available and include screening of libraries of existing or newly generated insertion mutant plant lines, whereby pools of genomic DNA of these mutant plant lines are subjected to PCR amplification using primers specific for the inserted DNA fragment and primers specific for the genomic region or allele, wherein the insertion is expected (see e.g. Maes et al., 1999, Trends in Plant Science, 4, pp 90-96). Thus, methods are available in the art to identify plant cells and plant lines comprising a mutation in the GTR gene. This population of mutant cells or plant lines can then be tested for functional GTR activity and GSL content and compared to non-mutated cells or plant lines with similar genetic background.

Further provided are plants obtainable by the above described methods and parts and products thereof, including seed, seed meal, seed oil, green plant tissue, such as rosette leaves, cauline leaves en silique walls, and root tissue, as well as uses thereof, for example, in animal feed, in pest management, such as biofumigation, and in cancer-prevention.

According to a particular embodiment of the invention, the transformed or mutated plant cells and plants obtained by the methods of the invention may contain, respectively, at least one other or at least one chimeric gene containing a nucleic acid encoding a protein of interest. Examples of such proteins of interest include an enzyme for resistance to a herbicide, such as the bar or pat enzyme for tolerance to glufosinate-based herbicides (EP 0 257 542, WO 87/05629 and EP 0 257 542, White et al. 1990), the EPSPS enzyme for tolerance to glyphosate-based herbicides such as a double-mutant corn EPSPS enzyme (U.S. Pat. No. 6,566,587 and WO 97/04103), or the HPPD enzyme for tolerance to HPPD inhibitor herbicides such as isoxazoles (WO 96/38567).

The transformed or mutated plant cells and plants obtained by the methods of the invention may be further used in breeding procedures well known in the art, such as crossing, selfing, and backcrossing. Breeding programs may involve crossing to generate an F1 (first filial) generation, followed by several generations of selfing (generating F2, F3, etc.). The breeding program may also involve backcrossing (BC) steps, whereby the offspring is backcrossed to one of the parental lines, termed the recurrent parent.

The transformed or mutated plant cells and plants obtained by the methods of the invention may also be further used in subsequent transformation procedures.

The following non-limiting examples describe the characteristics of plants obtained in accordance with the invention. Unless otherwise stated, all recombinant DNA techniques are carried out according to standard protocols as described in Sambrook et al. (1989) Molecular Cloning: A Laboratory Manual, Second Edition, Cold Spring Harbour Laboratory Press, NY and in Volumes 1 and 2 of Ausubel et al. (1994) Current Protocols in Molecular Biology, Current Protocols, USA. Standard materials and methods for plant molecular work are described in Plant Molecular Biology Labfax (1993) by R. D. D. Croy published by BIOS Scientific Publications Ltd (UK) and Blackwell Scientific Publications, UK.

In the description and examples, reference is made to the following sequences:
SEQ ID NO: 1: Coding DNA (without introns) of the GTR1 gene encoding a wild-type GTR1 protein from Arabidopsis thaliana.
SEQ ID NO: 2: wild type GTR1 protein encoded by SEQ ID NO: 1.
SEQ ID NO: 3: Coding DNA (without introns) of the GTR2 gene encoding a wild-type GTR2 protein from Arabidopsis thaliana.
SEQ ID NO: 4: wild type GTR2 protein encoded by SEQ ID NO: 3.
SEQ ID NO: 5: Coding DNA (without introns) of the GTR3 gene encoding a wild-type GTR3 protein from Arabidopsis thaliana.
SEQ ID NO: 6: wild type GTR3 protein encoded by SEQ ID NO: 5.
SEQ ID NO: 7: Coding DNA (without introns) of the GTR4 gene encoding a wild-type GTR4 protein from Arabidopsis thaliana.
SEQ ID NO: 8: wild type GTR4 protein encoded by SEQ ID NO: 7.
SEQ ID NO: 9: Coding DNA (without introns) of the GTR5 gene encoding a wild-type GTR5 protein from Arabidopsis thaliana.
SEQ ID NO: 10: wild type GTR5 protein encoded by SEQ ID NO: 9.
SEQ ID NO: 11: Coding DNA (without introns) of the GTR6 gene encoding a wild-type GTR6 protein from Arabidopsis thaliana.
SEQ ID NO: 12: wild type GTR6 protein encoded by SEQ ID NO: 11.
SEQ ID NO: 13: Coding DNA (with introns) of the GTR1-A1 gene encoding a wild-type GTR1-A1 protein from Brassica napus.
SEQ ID NO: 14: wild type GTR1-A1 protein encoded by SEQ ID NO: 13.
SEQ ID NO: 15: Coding DNA (with introns) of the GTR1-A2 gene encoding a wild-type GTR1-A2 protein from Brassica napus.
SEQ ID NO: 16: wild type GTR1-A2 protein encoded by SEQ ID NO: 15.
SEQ ID NO: 17: Coding DNA (with introns) of the GTR1-A3 gene encoding a wild-type GTR1-A3 protein from Brassica napus.
SEQ ID NO: 18: wild type GTR1-A3 protein encoded by SEQ ID NO: 17.
SEQ ID NO: 19: Coding DNA (with introns) of the GTR1-C1 gene encoding a wild-type GTR1-C1 protein from Brassica napus.
SEQ ID NO: 20: wild type GTR1-C1 protein encoded by SEQ ID NO: 19.
SEQ ID NO: 21: Coding DNA (with introns) of the GTR1-C2 gene encoding a wild-type GTR1-C2 protein from Brassica napus.
SEQ ID NO: 22: wild type GTR1-C2 protein encoded by SEQ ID NO: 21.
SEQ ID NO: 23: Coding DNA (with introns) of the GTR1-C3 gene encoding a wild-type GTR1-C3 protein from Brassica napus.
SEQ ID NO: 24: wild type GTR1-C3 protein encoded by SEQ ID NO: 23.
SEQ ID NO: 25: Coding DNA (with introns) of the GTR2-A1 gene encoding a wild-type GTR2-A1 protein from Brassica napus.
SEQ ID NO: 26: wild type GTR2-A1 protein encoded by SEQ ID NO: 25.
SEQ ID NO: 27: Coding DNA (with introns) of the GTR2-A2 gene encoding a wild-type GTR2-A2 protein from Brassica napus.
SEQ ID NO: 28: wild type GTR2-A2 protein encoded by SEQ ID NO: 27.
SEQ ID NO: 29: Coding DNA (with introns) of the GTR2-A3 gene encoding a wild-type GTR2-A3 protein from Brassica napus.
SEQ ID NO: 30: wild type GTR2-A3 protein encoded by SEQ ID NO: 29.
SEQ ID NO: 31: Coding DNA (with introns) of the GTR2-C1 gene encoding a wild-type GTR2-C1 protein from Brassica napus.
SEQ ID NO: 32: wild type GTR2-C1 protein encoded by SEQ ID NO: 31.
SEQ ID NO: 33: Coding DNA (with introns) of the GTR2-C2 gene encoding a wild-type GTR2-C2 protein from Brassica napus (partial sequence).
SEQ ID NO: 34: wild type GTR2-C2 protein encoded by SEQ ID NO: 33 (partial sequence).
SEQ ID NO: 35: Coding DNA (with introns) of the GTR2-C3 gene encoding a wild-type GTR2-C3 protein from Brassica napus.
SEQ ID NO: 36: wild type GTR2-C3 protein encoded by SEQ ID NO: 35.
SEQ ID NO: 37: Coding DNA (with introns) of the GTR1-A1 gene encoding a wild-type GTR1-A1 protein from Brassica rapa.
SEQ ID NO: 38: wild type GTR1-A1 protein encoded by SEQ ID NO: 37.
SEQ ID NO: 39: Coding DNA (with introns) of the GTR1-A2 gene encoding a wild-type GTR1-A2 protein from Brassica rapa.
SEQ ID NO: 40: wild type GTR1-A2 protein encoded by SEQ ID NO: 39.
SEQ ID NO: 41: Coding DNA (with introns) of the GTR1-A3 gene encoding a wild-type GTR1-A3 protein from Brassica rapa.
SEQ ID NO: 42: wild type GTR1-A3 protein encoded by SEQ ID NO: 41.
SEQ ID NO: 43: Coding DNA (with introns) of the GTR2-A1 gene encoding a wild-type GTR2-A1 protein from Brassica rapa.
SEQ ID NO: 44: wild type GTR2-A1 protein encoded by SEQ ID NO: 43.
SEQ ID NO: 45: Coding DNA (with introns) of the GTR2-A2 gene encoding a wild-type GTR2-A2 protein from Brassica rapa.
SEQ ID NO: 46: wild type GTR2-A2 protein encoded by SEQ ID NO: 45.
SEQ ID NO: 47: Coding DNA (with introns) of the GTR2-A3 gene encoding a wild-type GTR2-A3 protein from Brassica rapa.
SEQ ID NO: 48: wild type GTR2-A3 protein encoded by SEQ ID NO: 47.
SEQ ID NO: 49: Coding DNA (with introns) of the GTR1-C1 gene encoding a wild-type GTR1-C1 protein from Brassica oleracea.
SEQ ID NO: 50: wild type GTR1-C1 protein encoded by SEQ ID NO: 49.
SEQ ID NO: 51: Coding DNA (with introns) of the GTR1-C2 gene encoding a wild-type GTR1-C2 protein from Brassica oleracea.
SEQ ID NO: 52: wild type GTR1-C2 protein encoded by SEQ ID NO: 51.
SEQ ID NO: 53: Coding DNA (with introns) of the GTR1-C3 gene encoding a wild-type GTR1-C3 protein from Brassica oleracea.
SEQ ID NO: 54: wild type GTR1-C3 protein encoded by SEQ ID NO: 53.
SEQ ID NO: 55: Coding DNA (with introns) of the GTR2-C1 gene encoding a wild-type GTR2-C1 protein from Brassica oleracea.
SEQ ID NO: 56: wild type GTR2-C1 protein encoded by SEQ ID NO: 55.
SEQ ID NO: 57: Coding DNA (with introns) of the GTR2-C2 gene encoding a wild-type GTR2-C2 protein from Brassica oleracea.
SEQ ID NO: 58: wild type GTR2-C2 protein encoded by SEQ ID NO: 57.
SEQ ID NO: 59: Coding DNA (with introns) of the GTR2-C3 gene encoding a wild-type GTR2-C3 protein from Brassica oleracea.
SEQ ID NO: 60: wild type GTR2-C3 protein encoded by SEQ ID NO: 59.
SEQ ID NO: 61: Coding DNA (with introns) of the GTR1 gene encoding a wild-type
GTR1 protein from Arabidopsis thaliana.
SEQ ID NO: 62: wild type GTR1 protein encoded by SEQ ID NO: 61.
SEQ ID NO: 63: Coding DNA (with introns) of the GTR2 gene encoding a wild-type
GTR2 protein from Arabidopsis thaliana.
SEQ ID NO: 64: wild type GTR2 protein encoded by SEQ ID NO: 63.
SEQ ID NO: 65: Coding DNA (with introns) of the GTR2-A2 gene encoding a wild-type GTR2-A2 protein from Brassica rapa ecotype pekinensis.
SEQ ID NO: 66: wild type GTR2-A2 protein encoded by SEQ ID NO: 65.
SEQ ID NO: 67: Primer BrGTR2-A2-f (uracil at position 8)
SEQ ID NO: 68: Primer BrGTR2-A2-r (uracil at position 8)
SEQ ID NO: 69: Primer BrGTR2-A2-Till-f
SEQ ID NO: 70: Primer BrGTR2-A2-Till-r
SEQ ID NO: 71: Primer BrGTR2-A2-Inner-fw
SEQ ID NO: 72: Primer BrGTR2-A2-Inner-fw2
SEQ ID NO: 73: Primer BrGTR2-A2-Inner-rv
SEQ ID NO: 74: Primer BrGTR2-A2-126-f (uracil at position 10)
SEQ ID NO: 75: Primer BrGTR2-A2-126-r (uracil at position 10)
SEQ ID NO: 76: Primer BrGTR2-A2-145-f (uracil at position 9)
SEQ ID NO: 77: Primer BrGTR2-A2-145-r (uracil at position 9)
SEQ ID NO: 78: Primer BrGTR2-A2-192-f (uracil at position 9)
SEQ ID NO: 79: Primer BrGTR2-A2-192-r (uracil at position 9)
SEQ ID NO: 80: Primer BrGTR2-A2-229-f (uracil at position 11)
SEQ ID NO: 81: Primer BrGTR2-A2-229-r (uracil at position 11)
SEQ ID NO: 82: Primer BrGTR2-A2-359-f (uracil at position 10)
SEQ ID NO: 83: Primer BrGTR2-A2-359-r (uracil at position 10)
SEQ ID NO: 84: Primer AtGTR2f (uracil at position 8)
SEQ ID NO: 85: Primer AtGTR2r (uracil at position 8)
SEQ ID NO: 86: Primer AtGTR4e1f (uracil at position 8)
SEQ ID NO: 87: Primer AtGTR4e1r (uracil at position 9)
SEQ ID NO: 88: Primer AtGTR4e2f (uracil at position 9)
SEQ ID NO: 89: Primer AtGTR4e2r (uracil at position 13)
SEQ ID NO: 90: Primer AtGTR4e3f (uracil at position 13)
SEQ ID NO: 91: Primer AtGTR4e3r (uracil at position 9)
SEQ ID NO: 92: Primer AtGTR4e4f (uracil at position 9)
SEQ ID NO: 93: Primer AtGTR4e4r (uracil at position 8)
SEQ ID NO: 94: Primer AtGTR5f (uracil at position 8)
SEQ ID NO: 95: Primer AtGTR5r (uracil at position 8)
SEQ ID NO: 96: Primer T7
SEQ ID NO: 97: Primer pNB1rev
SEQ ID NO: 98: Primer AtGTR1_N879742_RP
SEQ ID NO: 99: Primer AtGTR1_N879742_RP
SEQ ID NO: 100: Primer AtGTR1_N870210_RP
SEQ ID NO: 101: Primer AtGTR1_N870210_RP
SEQ ID NO: 102: Primer AtGTR1_N409421_RP
SEQ ID NO: 103: Primer AtGTR1_N409421_RP
SEQ ID NO: 104: Primer AtGTR1_RP_fw
SEQ ID NO: 105: Primer AtGTR1_RP_rev
SEQ ID NO: 106: Primer AtGTR2_RP_fw
SEQ ID NO: 107: Primer AtGTR2_RP_rev
SEQ ID NO: 108: Primer AtGTR3_RP_fw
SEQ ID NO: 109: Primer AtGTR3_RP_rev
SEQ ID NO: 110: Primer AtGTR1pf
SEQ ID NO: 111: Primer AtGTR1pr
SEQ ID NO: 112: Primer AtGTR2pf
SEQ ID NO: 113: Primer AtGTR2pr
SEQ ID NO: 114: Primer AtGTR1r-YFP
SEQ ID NO: 115: Primer YFPf-AtGTR1-fusion
SEQ ID NO: 116: Primer YFPr-AtGTR1-3′ utr-fusion
SEQ ID NO: 117: Primer AtGTR1(3′ utr)f-YFP fusion
SEQ ID NO: 118: Primer AtGTR1(3′ utr)r
SEQ ID NO: 119: Coding DNA (with introns) of the GTR2-A1 gene encoding a wild-type GTR2-A1 protein from Brassica juncea.
SEQ ID NO: 120: wild type GTR2-A1 protein encoded by SEQ ID NO: 119.
SEQ ID NO: 121: Coding DNA (with introns) of the GTR2-A2 gene encoding a wild-type GTR2-A2 protein from Brassica juncea.
SEQ ID NO: 122: wild type GTR2-A2 protein encoded by SEQ ID NO: 121.
SEQ ID NO: 123: Coding DNA (with introns) of the GTR2-A3 gene encoding a wild-type GTR2-A3 protein from Brassica juncea.
SEQ ID NO: 124: wild type GTR2-A3 protein encoded by SEQ ID NO: 123.
SEQ ID NO: 125: Coding DNA (with introns) of the GTR2-B1 gene encoding a wild-type GTR2-B1 protein from Brassica juncea (partial sequence from exon 2 on).
SEQ ID NO: 126: wild type GTR2-B1 protein encoded by SEQ ID NO: 125 (partial sequence).
SEQ ID NO: 127: Coding DNA (with introns) of the GTR2-B2 gene encoding a wild-type GTR2-B2 protein from Brassica juncea.
SEQ ID NO: 128: wild type GTR2-B2 protein encoded by SEQ ID NO: 127.
SEQ ID NO: 129: Coding DNA (with introns) of the GTR2-B3 gene encoding a wild-type GTR2-B3 protein from Brassica juncea.
SEQ ID NO: 130: wild type GTR2-B3 protein encoded by SEQ ID NO: 129.
SEQ ID NO: 131: Coding DNA (without introns) of the GTR2-A1 gene encoding a wild-type GTR2-C1 protein from Brassica juncea.
SEQ ID NO: 132: Coding DNA (without introns) of the GTR2-A2 gene encoding a wild-type GTR2-A2 protein from Brassica juncea.
SEQ ID NO: 133: Coding DNA (without introns) of the GTR2-A3 gene encoding a wild-type GTR2-A3 protein from Brassica juncea.
SEQ ID NO: 134: Coding DNA (without introns) of the GTR2-B1 gene encoding a wild-type GTR2-B1 protein from Brassica juncea.
SEQ ID NO: 135: Coding DNA (without introns) of the GTR2-B2 gene encoding a wild-type GTR2-B2 protein from Brassica juncea.
SEQ ID NO: 136: Coding DNA (without introns) of the GTR2-B3 gene encoding a wild-type GTR2-B3 protein from Brassica juncea.
SEQ ID NO: 137: Coding DNA (with introns) of the GTR1-C1 gene encoding a wild-type GTR1-C1 protein from Brassica napus (variant).
SEQ ID NO: 138: dsRNA construct for downregulation of the expression of GTR1 expression in Brassica napus.
SEQ ID NO: 139: dsRNA construct for downregulation of the expression of GTR2 expression in Brassica napus
SEQ ID NO: 140: dsRNA construct for downregulation of the expression of GTR1 and GTR2 expression in Brassica napus.
SEQ ID NO: 141: Coding DNA (without introns) of the GTR1 gene encoding a wild-type GTR1 protein from Arabidopsis thaliana including the 30 amino acids NH2-terminal extension
SEQ ID NO: 142: wild type GTR1 protein including 30 amino acids N-terminal extension encoded by SEQ ID NO: 141
SEQ ID NO: 143: Coding DNA (without introns) of the GTR1-A1 gene encoding a wild-type GTR1A1 protein from Brassica napus including the 23 amino acids NH2-terminal extension
SEQ ID NO: 144: wild type GTR1-A1 protein including 23 amino acids N-terminal extension encoded by SEQ ID NO: 143
SEQ ID NO: 145: Coding DNA (without introns) of the GTR1-C1 gene encoding a wild-type GTR1 protein from Brassica napus including the 23 amino acids NH2-terminal extension
SEQ ID NO: 146: wild type GTR1-C1 protein including 23 amino acids N-terminal extension encoded by SEQ ID NO: 145
SEQ ID NO: 147: Coding DNA (without introns) of the GTR1-A1 gene encoding a wild-type GTR1-A1 protein from Brassica rapa including the 23 amino acids NH2-terminal extension
SEQ ID NO: 148: wild type GTR1-A1 protein including 23 amino acids N-terminal extension encoded by SEQ ID NO: 147
SEQ ID NO: 149: Coding DNA (without introns) of the GTR1-C1 gene encoding a wild-type GTR1-C1 protein from Brassica oleracea including the 23 amino acids NH2-terminal extension.
SEQ ID NO: 150: wild type GTR1-C1 protein including 23 amino acids N-terminal extension encoded by SEQ ID NO: 149.

Unless stated otherwise in the Examples, all recombinant DNA techniques are carried out according to standard molecular biological techniques as described in Sambrook and Russell (2001) Molecular Cloning: A Laboratory Manual, Third Edition, Cold Spring Harbor Laboratory Press, NY, in Volumes 1 and 2 of Ausubel et al. (1994) Current Protocols in Molecular Biology, Current Protocols, USA and in Volumes I and II of Brown (1998) Molecular Biology LabFax, Second Edition, Academic Press (UK). Standard materials and methods for plant molecular work are described in Plant Molecular Biology Labfax (1993) by R. D. D. Croy, jointly published by BIOS Scientific Publications Ltd (UK) and Blackwell Scientific Publications, UK. Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### EXAMPLES

Any methods of the invention not specifically described below may be performed by one of ordinary skill in the art without undue burden in the light of the disclosure herein.

### Example 1

### Identification and Characterization of GTR Proteins

Identification and Characterization of Arabidopsis GTR Sequences

A library of Arabidopsis transporters available as full length cDNAs from the RIKEN bioresource center (Ibaraki, Japan) was functionally screened in pools of ten genes for uptake of the most abundant Arabidopsis GSL 4-methylthiobutyl glucosinolate (4-MTB) into Xenopus oocytes. The constructed library consists of 239 transporters categorized as “organic solute transporters” or as “unknown function” with 10-14 transmembrane segments in the Arabidopsis membrane protein library database (Nour-Eldin et al., 2006, Plant Methods 2 : 17). As intended transport protein target in this study were potential importers from the apoplast, Xenopus oocytes uptake assays were performed in an acidic buffer (pH 5). Following this procedure At3g47960 (named herein AtGTR1; SEQ ID NO: 1, 2, 61 and 62) and At1g18880 (named herein AtGTR3; SEQ ID NO: 5 and 6) were identified as GSL transporters (Nour-Eldin, 2007, supra).

At3g47960 and At1g18880 belong to the NRT/PTR transporter family (Steiner et al., 1995, Molecular Microbiology 16: 825-834; Tsay et al., 2007, FEBS letters 581: 2290-2300), which has been shown to consist of nitrate, nitrite and peptide transporters (Tsay et al., 2007, FEBS letters 581: 2290-2300; Segonzac et al., 2007, Plant Cell 19 : 3760-3777; Komarova et al., 2008, Plant Physiol 148: 856-869). Phylogenetic analysis showed that these two genes form a small subclade with six homologs (see FIG. 1a): At3g47960 (named herein AtGTR1; SEQ ID NO: 1, 2, 61 and 62), At5g62680 (named herein AtGTR2; SEQ ID NO: 3, 4, 63 and 64), At1g18880 (named herein AtGTR3; SEQ ID NO: 5 and 6), At1g69860 (named herein AtGTR4; SEQ ID NO: 7 and 8), At1g69870 (named herein AtGTR5 or NRT1.7; SEQ ID NO: 9 and 10), At1g27080 (named herein AtGTR6 or NRT1.6; SEQ ID NO: 11 and 12). FIG. 1b and Table 4 indicate the sequence identity between the different AtGTR sequences.

TABLE 4
Sequence identity (in %) between Arabidopsis GTR1(without the 30 N-terminal AA i.e. as represented
in SEQ ID No 2), 2, 3, 4, 5 and 6 amino acid sequences/nucleic acid (coding sequences without
introns) sequences (the respective SEQ ID NO: numbers are indicated between brackets)

| | AtGTR1 | AtGTR2 | AtGTR3 | AtGTR4 | AtGTR5 | AtGTR6 |
|---|---|---|---|---|---|---|
| | (SEQ ID 2/1) | (SEQ ID 4/3) | (SEQ ID 6/5) | (SEQ ID 8/7) | (SEQ ID 10/9) | (SEQ ID 12/11) |
| AtGTR1 (2/1) | 100/100 | 78/75 | 60/62 | 34/46 | 42/50 | 38/49 |
| AtGTR2 (4/3) | | 100/100 | 60/63 | 33/46 | 43/50 | 37/49 |
| AtGTR3 (6/5) | | | 100/100 | 35/48 | 39/48 | 37/50 |
| AtGTR4 (8/7) | | | | 100/100 | 41/53 | 41/54 |
| AtGTR5 (10/9) | | | | | 100/100 | 63/67 |
| AtGTR6 (12/11) | | | | | | 100/100 |

Identification and Characterization of Brassica spp. GTR Sequences

Brassica napus, Brassica rapa, and Brassica oleracea GTR1 and GTR2 nucleic and amino acid sequences were identified in silico by screening genomic databases for sequences essentially similar to the Arabidopsis GTR1 and GTR2 sequences identified above. The Brassica GTR1 sequences were named GTR1-(A/C)1, 2 and 3 (see sequence listing SEQ ID NO: 13-24, SEQ ID NO: 37-42, SEQ ID NO: 49-54 and SEQ ID NO: 61-62) according to their decreasing similarity to the Arabidopsis GTR1 homolog (Table 5a and b). Similarly, the Brassica GTR2 sequences were named GTR2-(A/C)1, 2 and 3 (see sequence listing SEQ ID NO: 25-36, SEQ ID NO: 43-48, SEQ ID NO: 55-60 and SEQ ID NO: 63-66) according to their decreasing similarity to the Arabidopsis GTR2 homolog (Table 6a and b). B. juncea nucleotide and amino acid sequences are provided in SEQ ID NOs: 119-136.

TABLE 5a
Sequence identity (in %) between Arabidopsis (At), Brassica rapa (Br),
Brassica oleracea (Bo), Brassica napus (Bn) GTR1 amino acid sequences (as
determined by ClustalW - the SEQ ID NO: numbers are indicated between brackets) (using
the short GTR1 of Arabidopsis thaliana as represented in SEQ ID No. 2)

| | At | Br | Br | Br | Bo | Bo | Bo | Bn | Bn | Bn | Bn | Bn | Bn |
|---|---|---|---|---|---|---|---|---|---|---|---|---|---|
| | (2) | (38) | (40) | (42) | (50) | (52) | (54) | (14) | (16) | (18) | (20) | (22) | (24) |
| At (2) | 100 | 84 | 83 | 81 | 83 | 83 | 81 | 83 | 83 | 81 | 83 | 83 | 81 |
| Br (38) | | 100 | 89 | 83 | 99 | 88 | 83 | 98 | 89 | 84 | 98 | 88 | 83 |
| Br (40) | | | 100 | 84 | 88 | 99 | 84 | 88 | 100 | 85 | 88 | 99 | 84 |
| Br (42) | | | | 100 | 83 | 84 | 97 | 83 | 84 | 99 | 83 | 84 | 97 |
| Bo (50) | | | | | 100 | 88 | 83 | 98 | 88 | 83 | 99 | 88 | 83 |
| Bo (52) | | | | | | 100 | 83 | 88 | 99 | 84 | 88 | 100 | 84 |
| Bo (54) | | | | | | | 100 | 83 | 84 | 97 | 83 | 83 | 99 |
| Bn (14) | | | | | | | | 100 | 88 | 83 | 98 | 88 | 83 |
| Bn (16) | | | | | | | | | 100 | 85 | 88 | 99 | 84 |
| Bn (18) | | | | | | | | | | 100 | 83 | 84 | 98 |
| Bn (20) | | | | | | | | | | | 100 | 88 | 83 |
| Bn (22) | | | | | | | | | | | | 100 | 84 |
| Bn (24) | | | | | | | | | | | | | 100 |

TABLE 5b
Sequence identity (in %) between Arabidopsis (At), Brassica rapa (Br), Brassica oleracea
(Bo), Brassica napus (Bn) GTR1 nucleic acid (coding -without introns) sequences (as
determined by ClustalW - the SEQ ID NO: numbers are indicated between brackets) (using the
short GTR1 of Arabidopsis thaliana as represented in SEQ ID No. 1)

| | At | Br | Br | Br | Bo | Bo | Bo | Bn | Bn | Bn | Bn | Bn | Bn |
|---|---|---|---|---|---|---|---|---|---|---|---|---|---|
| | (1) | (37) | (39) | (41) | (49) | (51) | (53) | (13) | (15) | (17) | (19) | (21) | (23) |
| At (1) | 100 | 84 | 83 | 83 | 84 | 83 | 83 | 84 | 84 | 83 | 84 | 83 | 83 |
| Br (37) | | 100 | 88 | 87 | 98 | 89 | 87 | 98 | 88 | 87 | 98 | 89 | 87 |
| Br (39) | | | 100 | 86 | 88 | 98 | 86 | 88 | 99 | 86 | 88 | 98 | 86 |
| Br (41) | | | | 100 | 87 | 86 | 97 | 86 | 86 | 99 | 86 | 86 | 98 |
| Bo (49) | | | | | 100 | 88 | 87 | 98 | 88 | 87 | 99 | 88 | 87 |
| Bo (51) | | | | | | 100 | 86 | 88 | 98 | 86 | 88 | 100 | 86 |
| Bo (53) | | | | | | | 100 | 86 | 86 | 98 | 87 | 86 | 99 |
| Bn (13) | | | | | | | | 100 | 88 | 86 | 98 | 88 | 86 |
| Bn (15) | | | | | | | | | 100 | 86 | 88 | 98 | 86 |
| Bn (17) | | | | | | | | | | 100 | 87 | 86 | 98 |
| Bn (19) | | | | | | | | | | | 100 | 88 | 86 |
| Bn (21) | | | | | | | | | | | | 100 | 86 |
| Bn (23) | | | | | | | | | | | | | 100 |

TABLE 6a
Sequence identity (in %) between Arabidopsis (At), Brassica rapa (Br),
Brassica oleracea (Bo), Brassica napus (Bn) GTR2 amino acid sequences (as
determined by ClustalW - the SEQ ID NO: numbers are indicated between brackets)

| | At | Br | Br | Br | Bo | Bo | Bo | Bn | Bn | Bn | Bn | Bn |
|---|---|---|---|---|---|---|---|---|---|---|---|---|
| | (4) | (44) | (46) | (48) | (56) | (58) | (60) | (26) | (28) | (30) | (32) | (36) |
| At (4) | 100 | 91 | 91 | 83 | 91 | 91 | 82 | 91 | 91 | 84 | 91 | 84 |
| Br (44) | | 100 | 94 | 85 | 99 | 94 | 84 | 99 | 94 | 87 | 99 | 86 |
| Br (46) | | | 100 | 86 | 94 | 99 | 85 | 94 | 99 | 88 | 94 | 87 |
| Br (48) | | | | 100 | 86 | 86 | 95 | 86 | 86 | 97 | 86 | 95 |
| Bo (56) | | | | | 100 | 94 | 85 | 99 | 94 | 87 | 100 | 87 |
| Bo (58) | | | | | | 100 | 85 | 94 | 99 | 88 | 94 | 87 |
| Bo (60) | | | | | | | 100 | 85 | 85 | 94 | 85 | 95 |
| Bn (26) | | | | | | | | 100 | 94 | 87 | 99 | 87 |
| Bn (28) | | | | | | | | | 100 | 88 | 94 | 87 |
| Bn (30) | | | | | | | | | | 100 | 87 | 97 |
| Bn (32) | | | | | | | | | | | 100 | 87 |
| Bn (36) | | | | | | | | | | | | 100 |

TABLE 6b
Sequence identity (in %) between Arabidopsis (At), Brassica rapa (Br), Brassica oleracea
(Bo), Brassica napus (Bn) GTR2 nucleic acid (coding -without introns) sequences (as
determined by ClustalW - the SEQ ID NO: numbers are indicated between brackets)

| | At | Br | Br | Br | Bo | Bo | Bo | Bn | Bn | Bn | Bn | Bn |
|---|---|---|---|---|---|---|---|---|---|---|---|---|
| | (3) | (43) | (45) | (47) | (55) | (57) | (59) | (25) | (27) | (29) | (31) | (35) |
| At (3) | 100 | 87 | 87 | 83 | 87 | 86 | 82 | 87 | 87 | 84 | 87 | 85 |
| Br (43) | | 100 | 89 | 86 | 98 | 89 | 85 | 98 | 89 | 88 | 97 | 88 |
| Br (45) | | | 100 | 86 | 89 | 97 | 86 | 89 | 98 | 87 | 89 | 88 |
| Br (47) | | | | 100 | 86 | 86 | 96 | 86 | 86 | 97 | 86 | 96 |
| Bo (55) | | | | | 100 | 89 | 85 | 98 | 89 | 88 | 98 | 88 |
| Bo (57) | | | | | | 100 | 86 | 89 | 97 | 87 | 90 | 88 |
| Bo (59) | | | | | | | 100 | 85 | 85 | 95 | 85 | 96 |
| Bn (25) | | | | | | | | 100 | 89 | 88 | 97 | 88 |
| Bn (27) | | | | | | | | | 100 | 87 | 89 | 88 |
| Bn (29) | | | | | | | | | | 100 | 88 | 97 |
| Bn (31) | | | | | | | | | | | 100 | 88 |
| Bn (35) | | | | | | | | | | | | 100 |

In addition, type 1 and 2 sequences of the GTR1 and GTR2 sequences (i.e. GTR1-A1, GTR1-C1, GTR1-A2, GTR1-C2, GTR2-A1, GTR2-C1, GTR2-A2 and GTR2-C2 sequences) were identified in a B. napus embryo transcriptome database indicating that these types of GTR1 and 2 sequences are expressed in B. napus embryo's.

Further, Brassica rapa GTR2-A2 nucleic and amino acid sequences (SEQ ID NO: 65 and 66 in the sequence listing) were identified by cloning cDNA isolated from B. rapa ecotype pekinensis leaves using primers BrGTR2-A2-f (SEQ ID NO: 67) and BrGTR2-A2-r (SEQ ID NO: 68).

### Example 2

### Functional Characterization of GTR Proteins

Functional Characterization of Arabidopsis and Brassica GTR Proteins

AtGTR1 to 5, BrGTR2-A2 and 12 other members from the Arabidopsis NRT/PTR family were tested individually for uptake activity of 4-MTB in Xenopus oocytes.

AtGTR1 and 2 exhibited 5-fold higher uptake activity to AtGTR3 whereas AtGTR4 and AtGTR5 showed 10% 4-MTB uptake activity compared to AtGTR1. Uptake activity of all other tested NRT/PTRs was indistinguishable from uninjected oocytes.

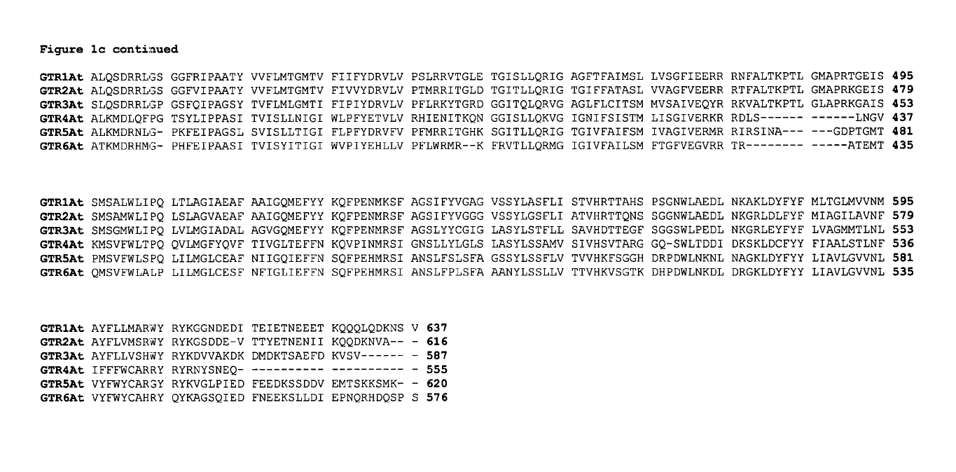

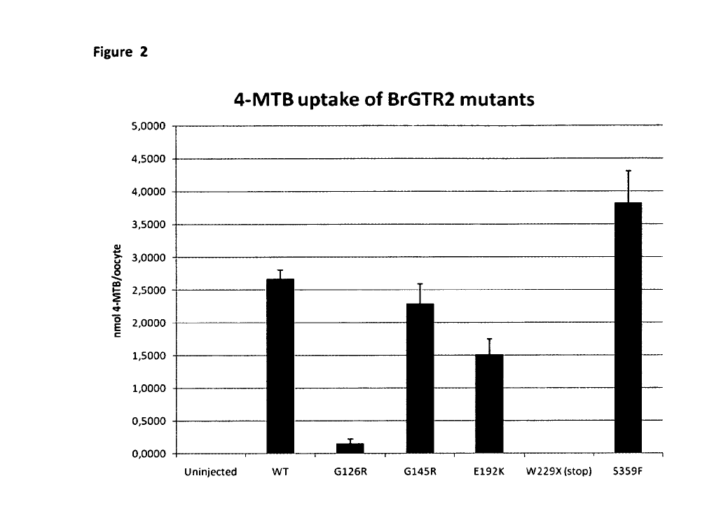

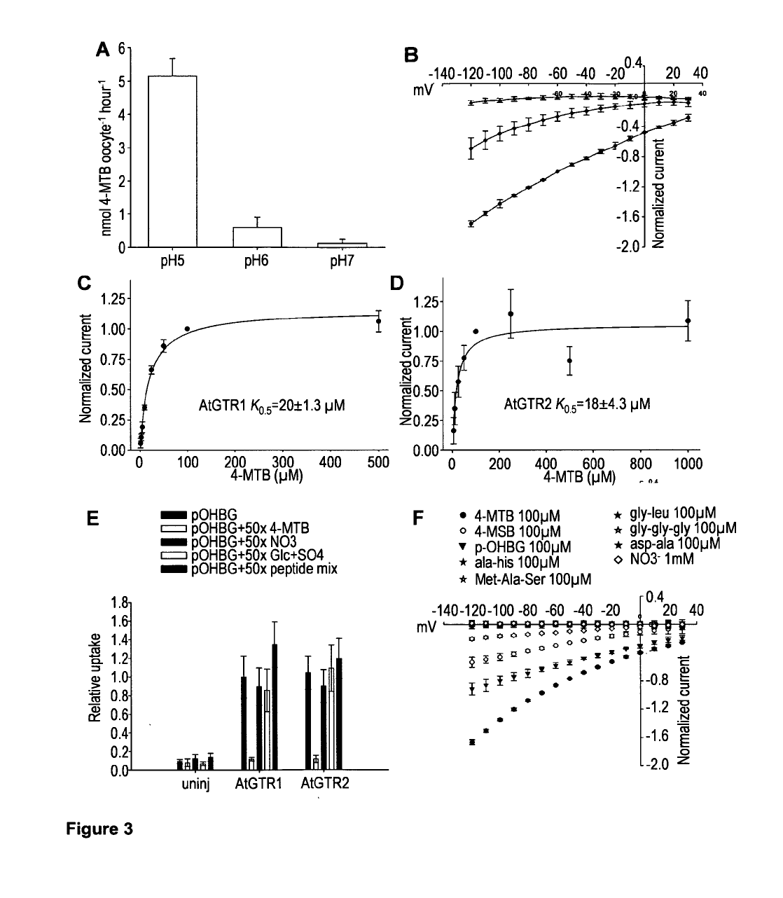

Oocytes expressing BrGTR2-A2 imported about 2.5 nmol 4-MTB per hour per oocyte (FIG. 2) while non-injected oocytes showed no detectable uptake of 4-MTB. In comparison, oocytes expressing AtGTR2 imported about 5 nmol 4-MTB/hour (FIG. 3A).

Biochemical Characterization of Arabidopsis Glucosinolate Transporter Proteins

AtGTR1 and AtGTR2, which showed the highest GSL uptake activity, were biochemically characterized in Xenopus oocytes. For both genes, lowering pH from 6 to 5 in the uptake medium resulted in an about 8-fold higher 4-MTB accumulation inside oocytes while uptake at pH 7 was indistinguishable from uninjected oocytes at pH 5 (FIG. 3A). This indicated that AtGTRs are proton:GSL symporters.

When current recordings were performed on oocytes expressing, respectively, AtGTR1 and AtGTR2 exposed to 100 μM 4-MTB at pH 5 and voltage clamped at −50 mV elicited inward currents in the 30 nA range. This indicates a net influx of positive ions through the AtGTRs upon exposure to GSLs. GSL-induced inward currents were voltage dependent increasing with hyperpolarizing membrane potential within the range of +30 to −120 mV (FIGS. 3B and F). As GSLs carry one negative charge per molecule the stochiometry of protons to GSLs must be at least 2:1, which appears to be a typical characteristic of proton-dependent oligopeptide transporter proteins (POTs) in general when transporting negatively charged substrates (Daniel et al., 2006, Physiology 21 : 93-102). At pH 5 clamped at −60 mV, import of 4-MTB by AtGTR1 and 2, respectively, followed Michaelis-Menten saturation kinetics displaying high affinity of AtGTR1 and ATGTR2 towards 4-MTB with Kₘ=20±1.2 μM and Kₘ=18.7±4.2 μM (FIG. 3C-D). This suggests that GSLs are likely to be physiological substrates of AtGTRs in planta. Remarkably, substrate dependent transport rates for AtGTR2 at 500 μM 4-MTB were 25% lower than at 250 μM and 1 mM. This observation was also noticeable in substrate dependent transport rates measured by direct LC-MS detection of GSL uptake.

The substrate specificity of the AtGTRs was investigated towards GSLs with varying sidechains. When AtGTR1 expressing oocytes clamped at −50 mV at pH 5 were perfused with 100 μM of the endogenous short chain methionine derived GSLs 4-MTB, 4-methylsulfinylbutyl-GSL and the exogenous phenylalanine derived p-hydroxybenzyl-GSL (pOHBG), an inward current in the magnitude of 30, 10 and nA for AtGTR1 was observed (FIG. 3F). Similarly AtGTR2 took up all three glucosinolates measured by LC-MS analysis of oocyte extracts (data not shown). This indicated that the transporters have broad specificity with respect to the GSL side-chain.

The substrate specificity investigations were extended to include substrates previously identified for the NRT/PTR transporter family namely di- and tripeptides and nitrate. Perfusing oocytes expressing AtGTR1 with 133 μM of the dipeptides, ala-his, gly-leu, asp-ala and the tripeptides gly-gly-gly and met-ala-ser at pH 5 did not result in any detectable currents. However, perfusing with 1 mM NO₃⁻ did result in currents but these amounted only to 1/10^{th} of currents measured when perfusing with 100 μM 4-MTB (FIG. 3F). This indicated that the AtGTRs possibly possess dual substrate specificities. To investigate this further, AtGTR1 and AtGTR2 expressing oocytes were exposed to 40 μM C¹⁴-labeled pOHBG in the presence of 50× excess unlabeled 4-MTB, NO₃⁻, and a mixture of di/tri peptides. Only 50× excess of 4-MTB reduced the uptake of pOHBG to background levels, while the other compounds did not have significant inhibitory effects (FIG. 3E).

Collectively, our biochemical analyses showed that AtGTR1 and AtGTR2 are high affinity, specific, electrogenic proton-driven GSL transporters with broad specificity towards a wide range of GSLs.

### Materials and Methods

### Example 2

Functional Screening of Transporter cDNA Library in Xenopus Oocytes:

Preparation and functional screening of a library of 239 Arabidopsis transporters in Xenopus oocytes was performed essentially as described in Nour-Eldin et al. (2006, Plant Methods 2 : 17) with two modifications. Firstly, DNA templates were not pooled prior to in vitro transcription. Instead, each PCR product was individually in vitro transcribed and then pooled into ten transcripts per pool. Individual transcription was performed to avoid unbalanced gene pools due to possible varying transcription efficiencies of pooled PCR fragments. Secondly, the library was divided into 23 pools containing 10 transcripts each, and a 24th pool containing 9 transcripts. Import assays were performed at room temperature in Kulori pH 5 containing 1 mM 4-MTB (for Arabidopsis sequences) or 0.5 mM 4-MTB (for Brassica sequences). Transcript pools which mediated GSL uptake into injected oocytes were subsequently injected as individual transcripts to identify GSL transporter.

Constructs for Expression in Xenopus Oocytes:

Coding sequences for AtGTR2 and AtGTR5 were cloned from cDNA from leaf tissue using primer pair AtGTR2f (SEQ ID NO: 84) and AtGTR2r (SEQ ID NO: 85) and primer pair AtGTR5f (SEQ ID NO: 94) and AtGTR5r (SEQ ID NO: 95), respectively (Nour-Eldin et al., 2006, Nucl Acids Res 34: e122). AtGTR4 was cloned by PCR amplification using primer pair AtGTR4e1f and AtGTR4e1r (SEQ ID NO: 86 and 87) for the first exon, primer pair AtGTR4e2f and AtGTR4e2r (SEQ ID NO: 88 and 89) for the second exon, primer pair AtGTR4e3f and AtGTR4e3r (SEQ ID NO: 90 and 91) for the third exon and primer pair AtGTR4e4f and AtGTR4e4r (SEQ ID NO: 92 and 93) for the fourth exon, and subsequent fusion of its four exons from genomic DNA. USER fusion and cloning into pNB1u were performed as described previously (Geu-Flores et al., 2007, Nucl Acids Res 35:e55).

Coding sequence for BrGTR2-A2 (SEQ ID NO: 65) was cloned from cDNA isolated from B. rapa ecotype pekinensis leaves. The CDS was cloned using primers BrGTR2-A2-f (SEQ ID NO: 67) and BrGTR2-A2-r (SEQ ID NO: 68) and USER cloned into the Xenopus expression vector pNB1u (Nour-Eldin et al., 2006, Nucl Acids Res 34: e122).

Linear templates for in vitro transcription were generated by PCR using primers T7 (SEQ ID NO: 96) and pNB1 rev (SEQ ID NO: 97). cRNA was In vitro transcribed as follows in 50 μl reaction volumes: 1-5 μg linear template was incubated at 37° C. in T7 RNA polymerase transcription buffer containing 80 U T7 RNA polymerase (Fermentas); 0.01 M DTT; 0.1 μg/μl BSA; 60 μM 3′-0-Me-m⁷G(5′)ppp(5′)G RNA Cap Structure Analog (New England Biolabs); 20 U Ribolock™ RNase inhibitor (Fermentas); 0.01 U pyrophosphatase (Fermentas); 1 mM rATP, rUTP, rCTP and 0.05 mM rGTP (LAROVA) for 30 min. rGTP was then added to a final concentration of 1 mM, and the reaction was incubated for 3 hours. RNA was purified by lithium chloride precipitation and dissolved in nuclease free H₂O to a final concentration of 0.5 μg/μl.

GSL Transport Assays in Xenopus Oocytes:

Oocyte Treatment and Injection:

Oocytes were prepared as described previously (1998, Methods Enzymol. 296: 17-52), and injected with 50 ng cRNA. Oocytes were incubated for 3-4 days at 17° C., before assaying for transport activity.

Assay Conditions:

Assays were performed in saline buffer kulori (90 mM NaCl, 1 mM KCl, 1 mM CaCl₂, 1 mM MgCl₂, 5 mM MES) adjusted to pH 5 with TRIS. Oocytes were pre-incubated in kulori buffer pH 5 for 5 min to ensure intracellular steady state pH, and subsequently transferred to 500 μl kulori pH 5 buffer containing indicated concentrations of substrates and incubated for 1 hour at room temperature. Oocytes were washed four times in ice cold kulori pH 5 buffer.

Transport Quantification:

For assays with radiolabeled substrates, single oocytes were ruptured in 100 μl 10% SDS in 4 ml scintillation vials by shaking for 20-30 minutes. 2.5 ml EcoScint™ scintillation fluid (National Diagnostics) was added, and imported compounds quantified by scintillation counting. For assays with unlabeled substrates, oocyte extracts were analysed by LC-MS. Oocytes were analyzed in batches of 5 oocytes per repetition. Washed oocytes were homogenized in 100 μl kulori pH 5 containing 1 mg/ml sulphatase (Sigma A-25-120) and left for 12 hours at room temperature. An equal volume of 100% methanol was then added and samples incubated at −20° C. for one hour and centrifuged for 15 min at 20000 g. Three μl supernatant containing the desulfated-glucosinolate was analyzed by analytical LC-MS using an Agilent 1100 Series LC (Agilent Technologies, Germany) coupled to a Bruker HCT-Ultra ion trap mass spectrometer (Bruker Daltonics, Bremen, Germany). A Zorbax SB-C18 column (Agilent; 1.8 mm, 2.1 mm×50 mm) was used at a flow rate of 0.2 ml/min, and the oven temperature was maintained at 35° C. The mobile phases were: A, water with 0.1% (v/v) HCOOH and 50 μM NaCl; B, acetonitrile with 0.1% (v/v) HCOOH. The gradient program was: 0 to 0.5 min, isocratic 2% B; 0.5 to 7.5 min, linear gradient 2 to 40% B; 7.5 to 8.5 min, linear gradient 40% to 90% B; 8.5 to 11.5 min isocratic 90% B; 11.6 to 15 min, isocratic 2% B. The flow rate was increased to 0.3 ml/min in the interval 11.2 to 13.5 min. The mass spectrometer was run in positive electrospray mode.

### Example 3

### Generation and Characterization of Plants and Plant Parts with Mutant GTR Genes

Generation and Characterization of Plants with Mutant GTR Genes

Arabidopsis T-DNA mutants of AtGTR1 (atgtr1-1), AtGTR2 (atgtr2-1) and AtGTR3 (atgtr3-1), respectively, were identified from the SALK T-DNA collection (Alonso et al., 2003, Science 301: 653-657). In silico genomic sequence analysis, showed that atgtr1-1 contains a T-DNA insertion in the first exon while atgtr2-1 contains a T-DNA insertion in the fourth exon of the coding regions and atgtr3-1 contains the T-DNA insertion in the third exon. Genotyping [using primer pair AtGTR1_N879742_RP (SEQ ID NO: 98) and AtGTR1_N879742_LP (SEQ ID NO: 99) for atgtr1, AtGTR2_N870210_RP (SEQ ID NO: 100) and AtGTR2_N870210_LP (SEQ ID NO: 101) for atgtr2; and AtGTR3_N409421_RP (SEQ ID NO: 102) and AtGTR3_N409421_LP (SEQ ID NO: 103) for atgtr3] and RT-PCR analysis [using primer pair AtGTR1_RT_fw (SEQ ID NO: 104) and AtGTR1_RT_rev (SEQ ID NO: 105) for AtGTR1, AtGTR2_RT_fw (SEQ ID NO: 106) and AtGTR2_RT_rev (SEQ ID NO: 107) for AtGTR2 and AtGTR3_RT_fw (SEQ ID NO: 108) and AtGTR3_RT_rev (SEQ ID NO: 109) for AtGTR3] showed that atgtr1, atgtr2 and atgtr3 homozygous mutants were null mutants for their respective AtGTR. A double knockout mutant was generated by crossing atgtr1 mutant plants to atgtr2 mutant plants. From the segregating F2 population, 3 homozygous lines were identified for each of the wild type, atgtr1, atgtr2 and atgtr1/atgtr2 genotypes.

Brassica rapa substitution mutants of BrGTR2-A2 were identified in a mutated B. rapa ecotype R-0-18 plant population (RevGenUK) by TILLING using the BrGTR2-A2 sequence of SEQ ID NO: 65 and tilling primers BrGTR2-A2-Till-f (SEQ ID NO: 69), BrGTR2-A2-Till-r (SEQ ID NO: 70), BrGTR2-A2-Inner-fw (SEQ ID NO: 71), BrGTR2-A2-Inner-fw2 (SEQ ID NO: 72), and BrGTR2-A2-Inner-rv (SEQ ID NO: 73). Twenty substitution mutations were found: 7 silent mutations (nucleic acid mutation resulting in codon encoding the same amino acid), 8 non-severe mutations (nucleic acid mutation resulting in codon encoding an amino acid belonging to the same functional class), 4 severe mutations (nucleic acid mutation resulting in codon encoding an amino acid belonging to a different functional class, e.g. small for large, nonpolar for polar, aliphatic for nonaliphatic, aromatic for nonaromatic, hydrophobic for polar, acidic side chain for nonacidic side chain) and 1 stop codon mutation. The nucleotide position of severe and STOP mutant codons found in the genomic DNA sequence of BrGTR2-A2 from B. rapa ecotype R-0-18 are indicated in Table 7 as well as the corresponding amino acid position in SEQ ID NO: 66. Codons encoding the indicated amino acids in BrGTR2-A2 from B. rapa ecotype pekinensis can be found at the indicated amino acid positions in SEQ ID NO: 65. Homozygous wildtype and mutants B. rapa plants were identified for each mutation using primers BrGTR2-A2-Inner-fw (SEQ ID NO: 71), BrGTR2-A2-Inner-fw2 (SEQ ID NO: 72), and BrGTR2-A2-Inner-rv (SEQ ID NO: 73) and sequencing of the obtained amplicons to determine the presence of a wildtype or mutant codon at the positions indicated in Table 7. Mutant plants are grown and GSL content is determined in different plant parts.

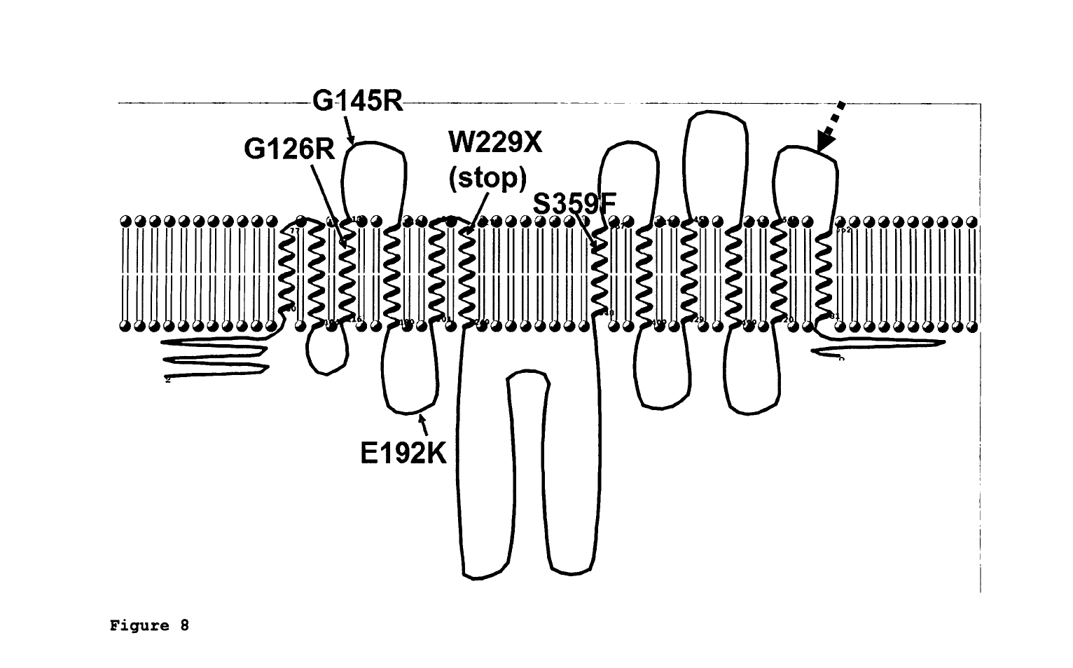

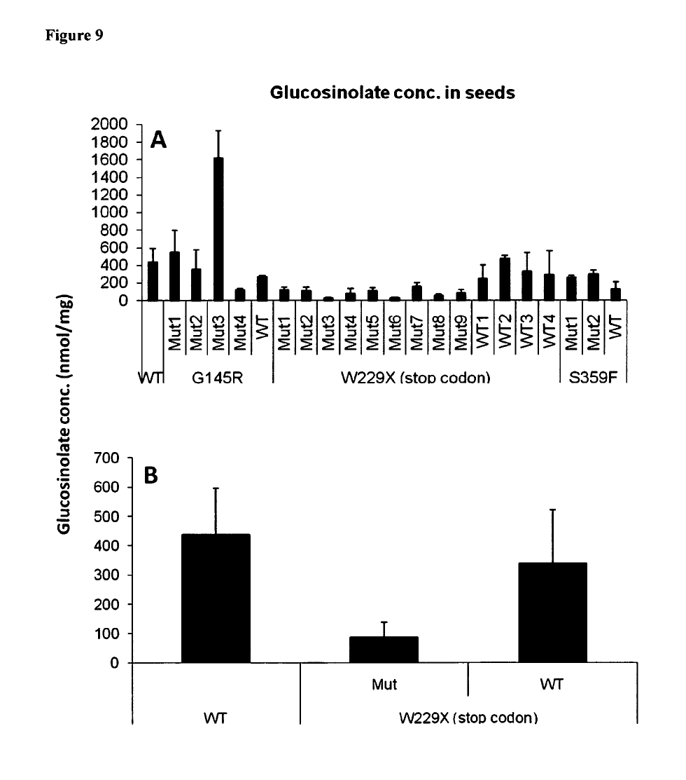

Mutant plants are grown and GSL content is determined in different plant parts. The results are summarized in FIG. 9. Seeds from Brgtr2 knockout mutants (BrGTR2 (W229X Mut1-9)) have reduced glucosinolate content, both compared to “true” WT B. rapa seeds as well as to BrGTR2 S(W229X) WTS. The reduction is ˜80% compared to BrGTR2 (W229X-WT) plants, when pooling seeds of each genotype. B. rapa plants containing BrGTR2 mutated in amino acid G145R (G145R Mut1-4) show highly variable glucosinolate content, which is probably caused by segregation of additional EMS-induced point mutations in genes affecting seed glucosinolate content.

TABLE 7
Substitution and STOP codon mutations in BrGTR2-A2 from B. rapa
ecotype R-0-18

| SEQ ID | | | | | |
|---|---|---|---|---|---|
| NO: of | | | | Amino acid | uptake |
| used | | | | position in | activity |
| tilling | Nucleotide | WT->mutant | WT->mutant | SEQ ID | in % |
| primers: | position | codon | amino acid | NO: 66 | of WT |
| 69 + 73 | 833 | AGG->AGA | Gly->Arg | 126 | 5% |
| 69 + 73 | 890 | GGA->AGA | Gly->Arg | 145 | 85% |
| 71 + 73 | 1031 | GAG->AAG | Glu->Lys | 192 | 56% |
| 71 + 73 | 1143 | TTG->TAG | Trp->STOP | 229 | 0% |
| 72 + 73 | 1608 | TCC->TTC | Ser->Phe | 359 | 143% |

Functional Characterization of Mutant Brassica rapa GTR2-A2 Proteins

The identified mutant BrGTR2-A2 proteins were tested individually for uptake activity of 4-MTB in Xenopus oocytes. Point mutations were made in BrGTR2-A2 by USER fusion as described previously (Geu-Flores et al., 2007, Nucl Acids Res 35:e55). Briefly, the coding sequence is PCR amplified as two fragments flanking the desired mutation location. Each fragment has complementary tails with the base substitution embedded. In addition each tail contains a uracil residue which upon cleavage with the uracil specific excision reagent (USER) yields a long single stranded overhang which readily and firmly anneals with the complementary tail from the other fragment. At the distal terminal of each fragment the BrGTR2-A2-f primer was used as the forward primer for the fragment lying upstream of the mutation and the BrGTR2-A2-r as the reverse primer for the fragment lying downstream of the mutation. Primers used at the junction are given in SEQ ID NO: 74 and 75 (forward and reverse primer for amino acid 126 substitution as indicated in Table 7), in SEQ ID NO: 76 and 77 (forward and reverse primer for amino acid 145 substitution as indicated in Table 7), in SEQ ID NO: 78 and 79 (forward and reverse primer for amino acid 192 substitution as indicated in Table 7), in SEQ ID NO: 80 and 81 (forward and reverse primer for amino acid 229 substitution as indicated in Table 7) and in SEQ ID NO: 82 and 83 (forward and reverse primer for amino acid 359 substitution as indicated in Table 7). Each clone was verified by sequencing after insertion into the pNB1u vector, RNA was made and injected into oocytes as described above. Uptake activity of each clone is given in FIG. 2 and Table 7.

In conclusion, BrGTR2 containing a stop codon was non-functional, indicating that the truncated form of the protein is nonfunctional in planta, whereas remaining mutations exhibited varying transport activity. These varying transport activities indicate that amino acid positions that were subject to substitution are important for BrGTR2 transport activity. According to the proposed model for POT proteins, transmembrane helices no. 1, 2, 4, 5, 7, 8, 10 and 11 line the central pore of the transporter and are involved in the transport mechanism. Remaining helices (no. 3, 6, 9 and 12) are found in the periphery and may confer structural stability. Mutations BrGTR2 (G126R) and BrGTR2 (S359F) are in transmembrane helix 3 and 7, respectively. Helix 3 should not be involved in the transport mechanism; therefore, it can be hypothesized that BrGTR2 (G126R) may have severely disrupted the protein tertiary structure, leading to almost no uptake activity. Helix 7 lines the central pore, where transport occurs; thus, the increased uptake activity seen for this BrGTR2 (S359F) could be due to the transition from a small side chain (serine) to a large side chain (phenylalanine), leading to an enlargement of the pore. The change in polarity of the S359F-transition may further contribute to the increased activity. Mutation BrGTR2 (G145R) causes no significant change in uptake of 4-MTB. Mutation BrGTR2 (E192K) in the cytoplasmic loop between helices 4 and 5 seems to cause a reduction in glucosinolate uptake. An amino acid shift from a negatively charged glutamic acid to a positively charged lysine is quite drastic and possibly may have disrupted protein structure. Furthermore, a phosphorylation site has been suggested on the peptide sequence “ser-glu-ser-gly-lys-arg” of AtGTR2. An alignment of AtGTR2 and BrGTR2 shows that this sequence is conserved and that the peptide corresponds to the residues 191-196 in BrGTR2. Thus, if one of the serines (191 and 193) surrounding the mutated glutamic acid (E192K) is normally phosphorylated (or dephosphorylated), this could be prevented by the transition to lysine; which could in turn reduce uptake activity.

Characterization of GSL Content in Seed of Mutant GTR Arabidopsis Plants

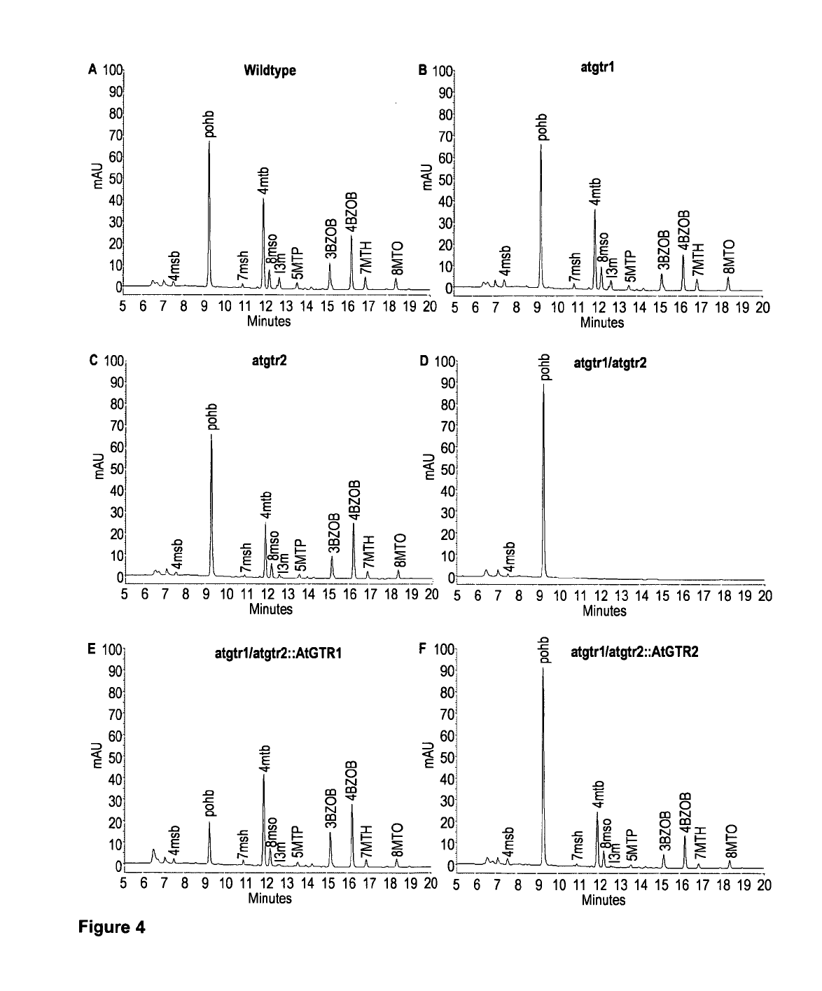

GSL analysis of segregating progeny from heterozygous atgtr1, atgtr2 and atgtr3 mutants showed that seeds from homozygous atgtr3 plants contain wild type levels of total aliphatic and indole GSLs. Similarly, atgtr1 had no significant reduction in total aliphatic and indole glucosinolate content per seed whereas atgtr2 seeds about 50% reduction in total aliphatic GSLs concentrations (FIGS. 4A-C and 5A). In addition, indole GSL content was not detectable in atgtr2 seeds. When analyzing 20 seeds per plant, GSL levels in seeds from each homozygous atgtr1/atgtr2 line were below detection limits, indicating a critical role for the combined activities of AtGTR1 and AtGTR2 in transporting GSLs into seeds. When analyzing 10 mg seeds from wildtype and atgtr1/atgtr2 plants, it was possible to detect trace amounts of glucosinolates in seeds from atgtr1/atgtr2 plants at 250 fold lower levels compared to wildtype.

For molecular complementation, respectively, a AtGTR1 construct including 2 kb promoter, the genomic sequence fused to YFP and 3′UTR and a AtGTR2 construct consisting of the 2 kb promoter followed by the genomic sequence were introduced into the atgtr1/atgtr2 double mutant and were shown to restore both aliphatic and indole GSL content in seeds (FIG. 4E-F). This demonstrates that the GSL-free seed phenotype in the atgtr1/atgtr2 mutant is due to the absence of AtGTR1 and AtGTR2 transport proteins.

Characterization of GSL Content in Leaves of Mutant GTR Arabidopsis Plants

GSL levels were measured in entire rosettes before bolting, after bolting and at senescence. In rosettes from wildtype plants, aliphatic GSL content was measured to, respectively, 28±13 and 68±19 nmol/rosette before and after bolting and to 8±3 nmol in senescent rosettes. In contrast, in rosettes from atgtr1/atgtr2 plants, aliphatic GSL content increased dramatically from 27±11 before bolting to 410±78 nmol/rosette after bolting and remained at 161±31 nmol/rosette at senescence. atgtr1 and atgtr2 single knockout line rosettes contained intermediate levels compared to the atgtr1/atgtr2 rosettes (FIG. 4B). In comparison to aliphatic GSLs, indole GSL content displayed a larger variation in rosette tissues. However, when comparing wildtype to atgtr1/atgtr2 rosettes after bolting and at senescence, atgtr1/atgtr2 rosettes contained in average 2 fold higher indole GSLs. Collectively, these observations indicate that rosette tissues continuously synthesize and export both aliphatic and indole GSLs and demonstrate that both AtGTR1 and AtGTR2 are critical for this activity in wildtype plants.

Characterization of GSL Content in Stems, Flowers, Leaves, Siliques and Silique Walls of Mutant GTR Arabidopsis Plants

From plants after bolting, GSL content was determined separately in inflorescences (without siliques and cauline leaves), cauline leaves, total intact siliques (including seeds) and silique walls from single dissected siliques (lowest and most mature). In addition, single silique walls were analysed at senescence.

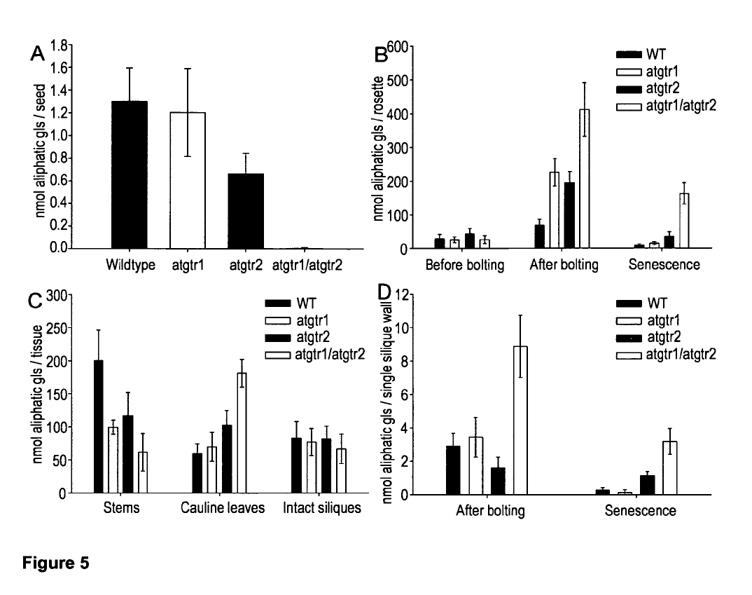

In wildtype plants after bolting, inflorescences minus siliques and cauline leaves contained 200±46 nmol aliphatic GSLs per inflorescence whereas inflorescences from atgtr1/atgtr2 plants contained 61±28 nmol and inflorescences atgtr1 and atgtr2 plants contained intermediate levels (FIG. 5C). In cauline leaves from wildtype plants after bolting, aliphatic GSL content amounted to 59±15 nmol per total cauline leaves while atgtr1/atgtr2 mutants contained 181±20 nmol in their cauline leaves (FIG. 5C). Increased contents were also observed for aliphatic GSLs in silique walls where concentrations in atgtr1/atgtr2 plants after bolting were 8.8±3 nmol per silique wall in contrast to 2.9±0.8 nmol/silique wall from wildtype plants. At senescence, GSL levels in silique walls from wildtype decreased to 0.3±0.13 nmol per silique wall whereas atgtr1/atgtr2 silique walls retained 3±0.78 nmols (FIG. 5D).

Collectively, these observations indicate that at this developmental stage, cauline leaves and silique walls in addition to rosette tissues function as GSL sources, whereas the entire inflorescence or parts thereof appears to constitute sinks for AtGTR1 and AtGTR2 mediated GSL transport. Two additional observations are noteworthy when analyzing siliques. Firstly, no significant differences were observed in GSL content when analyzing intact siliques from wildtype or atgrt1/atgtr2 mutants after bolting (FIG. 5C) which indicates that at this developmental stage GSL content of the intact siliques does not exclusively depend on the activity of AtGTR1 and AtGTR2 (FIG. 5D). Secondly, in contrast to the other tissues atgtr1 and atgtr2 single mutants after bolting did not exhibit an intermediate phenotype compared to atgtr1/atgtr2, on the contrary both atgtr1 and atgtr2 resembled wildtype possibly indicating an intriguing synergistic effect of mutating both transporters (FIG. 5D).

When calculating the balance of total aliphatic GSL content in the various tissues of the plants after bolting, atgtr1/atgtr2 plants is seen to contain an excess of about 460 nmol in rosettes and cauline leaves compared to wildtype. As this increase is only met by an about 160 nmol decrease in stems and intact siliques in atgtr1/atgtr2 plants, this results in an about 300 nmol increased content of total aliphatic GSLs per total aerial parts in atgtr1/atgtr2 plants compared to wildtype (Table 8).

TABLE 8
Aliphatic glucosinolate content per tissue (nmol/g) in soil grown plants
after bolting

| | Wildtype | atgtr1/atgtr2 |
|---|---|---|
| Rosette | 68 ± 19 | 410 ± 78 |
| Cauline leaves | 59 ± 15 | 181 ± 20 |
| Intact siliques | 83 ± 25 | 67 ± 22 |
| Stems | 200 ± 46 | 61 ± 28 |
| Balance | 398 ± 51 | 703 ± 75 |

Characterization of GSL Content in Roots of Mutant GTR Arabidopsis Plants

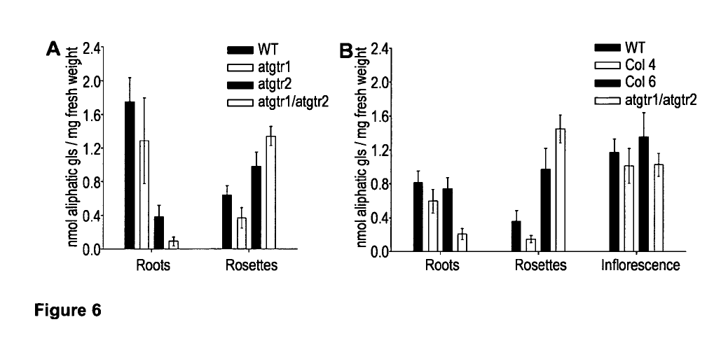

GSL concentrations were measured in roots, rosettes and inflorescence from hydroponically grown plants before and after bolting. Before bolting, aliphatic glucosinolate content in wildtype roots was 1.75±0.28 nmol/mg fresh weight, whereas atgtr1/atgtr2 roots contained 20 fold less (0.08±0.06 nmol/mg). After bolting, roots from wildtype plants contained 0.8±0.14 nmol/mg aliphatic glucosinolates whereas roots from atgtr1/atgtr2 plants contained 0.2±0.06 nmol/mg i.e. 4 fold less (FIG. 6A). In contrast to soil grown plants, aliphatic glucosinolate content in the corresponding rosettes from hydronically grown plants before bolting was 2-fold higher in atgtr1/atgtr2 plants (1.33±0.11 nm/mg) compared to wildtype (0.64±0.1 nmol/mg) (FIG. 6B). After bolting the fold difference between rosettes from wildtype and atgtr1/atgtr2 plants became more pronounced with wildtype plants containing 0.35±0.13 nmol/mg whereas atgtr1/atgtr2 rosettes contained 1.44±0.16 nmol/mg i.e. 4 fold higher concentrations (FIG. 6B). Lastly, no significant differences was seen in aliphatic glucosinolate concentration between inflorescences from wildtype and atgtr1/atgtr2 plants (FIG. 6B). Taken together these observations show that the increased glucosinolate accumulation in atgtr1/atgtr2 aerial tissues, to a large extent, can be accounted for by amounts otherwise transported to the roots. Roots thus constitute a strong and yet unidentified sink for glucosinolate transport.

Characterization of Tissue, Cellular and Subcellular Localization of AtGTR1&2 in Arabidopsis Plants

2 kb of the respective promoters were used to drive the expression of a beta-D-glucoronidase (GUS). Before bolting pGTR1::GUS activity was confined to distinct spots surrounding the hydathodes. In contrast, after bolting pGTR1::GUS activity was detected throughout the leaf including the vascular tissue, mesophyl and epidermis of rosette leaves. In developing flowers, pGTR1::GUS activity was found in individual cells in the epidermis of sepals, while developing siliques showed activity in the epidermis as well as in the funiculus. This expression pattern indicated that AtGTR1 may play a general role in transporting glucosinolates between cells throughout the leaf after bolting. The localization to the epidermis indicated that glucosinolates might be transported to the outer perimeter of leaves.

In pGTR2::NLS-GFP-GUS transgenic plants GUS activity was strongly and predominantly detected in the vascular tissue throughout development with some activity also detected in the epidermis of leaves. GUS activity was furthermore detected in the vasculature of sepals, petals and in the filaments of developing flowers. Expression was also detected in the vascular tissue of silique walls and the funiculus of developing seeds. In comparison, GUS activity could not be detected in any tissue of nontransformed plants at any developmental stage. The apparent confined and constitutive expression of AtGTR2 in the vascular tissue indicated a major role in transporting glucosinolates across barriers in the vascular tissue to enable long distance transport to sinks

### Materials and Methods

### Example 3

Plant Material and Growth Conditions:

For GSL analysis and expression localization studies in above ground tissues, Arabidopsis plants (ecotype Columbia-0) were cultivated on soil in 4 cm pots in growth chambers at 20° C., at 16 hour light (700 μE*m*⁻²*s⁻¹). For GSL analysis in roots, plants were grown in a hydroponic system as described in (Lehmann et al. 2009, Mol Plant 2: 390-406). Briefly, seeds were germinated on 0.5% plant agar/50% Gibeaut's nutrient solution (Gibeaut et al., 1997, Plant Physiol 115: 317-319) in 0.5 ml microfuge tubes held in an empty pipette-tip box. Approximately one week after germination, the bottoms of the tubes were cut off and the boxes filled with 100% nutrient solution. The plants were transferred to larger containers approximately two weeks later and were grown under a 16 h:8 h photoperiod, 20° C. Nutrient solution was changed weekly, and the genotypes were grown in separate containers. Harvesting of individual tissues for glucosinolate analysis was performed as described for soil-grown plants.

GSL Extraction and Analysis from Plant Tissues:

GSL levels were analyzed in a representative line (of three identified for each) for each genotype originating from the atgtr1-1xatgtr2-1 cross at three time points, 1) Before bolting: 3-week old plants (growth stage 1.10-1.12) characterized by a fully formed rosette with 10-14 leaves before emergence of inflorescence, 2) After bolting: 5 week old plants (growth stage 6.10-6.30) characterized by the lowest silique being at full length and the presence of at least 7-10 developing siliques, 3) Senescence: 8-9 week old plants (growth stage 9.7) characterized by senescent dry green tissues and all siliques developed containing mature seeds. Growth stages definitions are as described previously (Boyes et al., 2001, Plant cell 13: 1499-1510). GSL levels were also analyzed in the representative line for each genotype in rosettes, roots and inflorescence in hydroponically grown plants before and after bolting.

For analyses performed on plants before and after bolting grown in soil or hydroponically fresh tissue was harvested and homogenized. Specifically for analyses of silique walls after bolting intact siliques were freeze dried prior to dissection into silique walls and immature seeds. For analyses of senescent plants dry tissue were harvested and homogenized. For mature seed analysis 20 seeds were analyzed. GSLs were extracted and analyzed as desulfo GSLs as described previously (Hansen et al. 2007, Plant J 50:902-910) using an HP1200 Series HPLC from Agilent equipped with a C-18 reversed phase column (Supelcosil LC-18-DB, 25 cm×4.6 mm, 5 μm particle size, Supelco, Bellefonte, Pa., USA) by using a water (solvent A)-acetonitrile (solvent B) gradient at a flow rate of 1 ml min⁻¹ (injection volume 45 μl). The gradient was as follows: 1.5-7% B (5 min), 7-25% (6 min), 25-80% (4 min), 80% B (3 min), 80-35% B (2 min), 35-1.5% B (2 min), and 1.5% B (3 min). The eluent was monitored by diode array detection between 200 and 400 nm (2-nm interval). Desulfo-GSLs were identified based on comparison of retention times and UV absorption spectra with those of known standards (Reichelt et al., 2002). Results are given as nmols calculated relative to response factors (Brown et al., 2003; Fiebig and Arens, 1992).

Construction of Reporter and Complementation Constructs:

The E. coli strain DH10B was used for all cloning. For AtGTR1 and AtGTR2 promoter:reporter constructs, promoters were amplified from Arabidopsis (Col-0) genomic DNA, using primers AtGTR1 pf (SEQ ID NO: 110) and AtGTR1 pr (SEQ ID NO: 111) for the AtGTR1 promoter and primers AtGTR2pf (SEQ ID NO: 112) and AtGTR2pr (SEQ ID NO: 113) for the AtGTR2 promoter. Promoter fragments were USER cloned into pCambia3300u-NLS-GPF-GUS, upstream of a nuclear localization signal followed by GFP fused to beta-D-glucoronidase (Chytilova et al., 1999, Ann. Bot. 83: 645-654). For complementation construct AtGTR2 promoter-ATGTR2 genomic gene fragments was PCR amplified using primers AtGTR2pf (SEQ ID NO: 112) and AtGTR2pr (SEQ ID NO: 113) and USER cloned into pCambia1300u. For complementation construct AGTR1 promoter-AtGTR1 genomic gene-YFP-3′UTRs each fragment was PCR amplified using primers AtGTR1pf (SEQ ID NO: 110) and AtGTR1r-YFP (SEQ ID NO: 114) for promoter-genomic gene fragment, primers YFPf-AtGTR1-fusion (SEQ ID NO: 115) and YFPr-pot13′UTRfusion (SEQ ID NO: 116) for YFP fragment and primer AtGTR1(3′UTR)f-YFP fusion (SEQ ID NO: 117) and AtGTR1(3′UTR)r (SEQ ID NO: 118) for 3′UTR fragment. Fragments were USER fused into the pCambia1300u vector, Plant expression plasmids were used to transform Agrobacterium (GV3101) for stable Arabidopsis transformations.

Staining of Transgenic Arabidopsis Plants for GUS Activity:

X week old Arabidopsis GTR1 prom-GFP-GUS and GTR2 prom-GFP-GUS plants were submerged in a solution containing 1 mM X-Glc, 0.5% Triton X-100, 20% (v/v) methanol, 100 mM Tris-HCl (pH 7.5) and 10 mM ascorbate, and incubated at 37′C for 16 hours. Chlorophyll were cleared after staining by washing and incubation in 70% ethanol.

### Example 4

### Generation and Characterization of Mutant Brassica GTR Genes and Plants and Plant Parts Comprising them

Seeds from Brassica plants (M0 seeds) are exposed to EMS. M1 plants are grown from mutagenized seeds (M1 seeds) and selfed to generate M2 seeds. M2 plants are grown and DNA samples are prepared from plant samples. The DNA samples are screened for the presence of point mutations in the GTR genes causing the introduction of STOP codons in the protein-encoding regions of the GTR genes, the substitution of amino acids in the GTR proteins or splice site mutations by direct sequencing with GTR-specific primers as described above and analyzing the sequences for the presence of the point mutations. For each mutant GTR gene identified in the DNA sample of an M2 plant, M2 plants derived from the same M1 plant as the M2 plant comprising the GTR mutation are grown and DNA samples are prepared from plant samples of each individual M2 plant. The DNA samples are screened for the presence of the identified GTR point mutation.

The following mutant alleles in B. napus have been isolated and the total glucosinolate content was tested in M3 wet seeds:
a. GTR2-C2-ems02
b. GTR2-C1-ems05
c. GTR2-C1-ems01
d. GTR2-A2-ems09
e. GTR2-A2-ems03
f. GTR2-A1-ems01

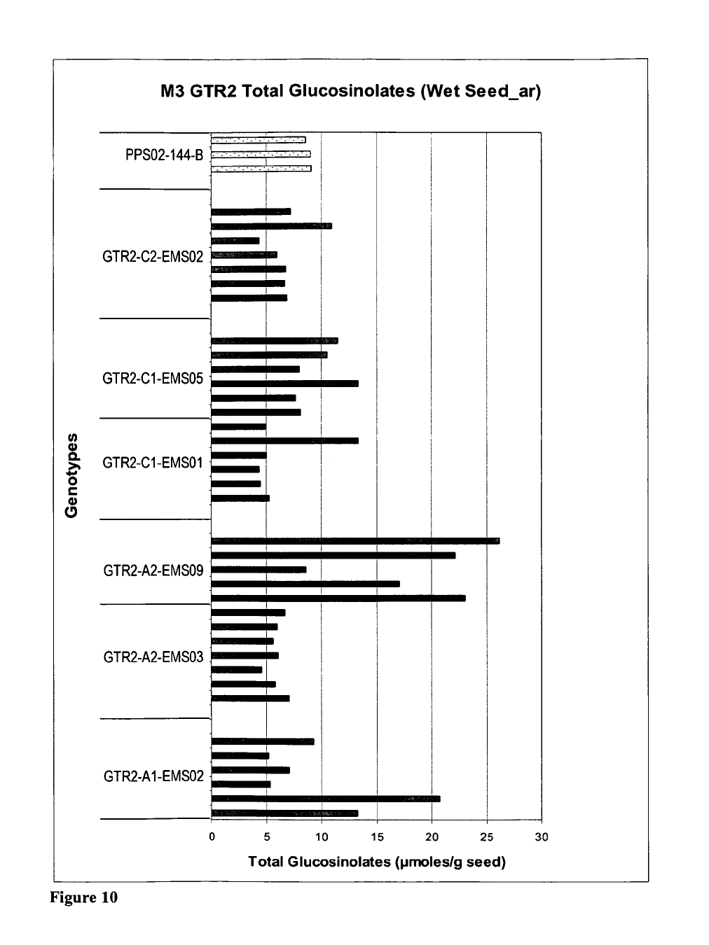

For more details concerning the mutant alleles see Tables 3a and 3b. The results are summarized in FIG. 10. The maximal reduction in total glucosinolates observed was 51% with single gene knock-outs. Each gene copy tested resulted in similar effects. The high level of variation may be related to the high number of unrelated background mutations in non-backcrossed material.

Mutant GTR alleles are thus generated and isolated. Also, plants comprising such mutant alleles can be used to combine selected mutant and/or wild type alleles in a plant, for example a Brassica breeding line, as follows: A Brassica plant containing a mutant GTR gene (donor plant line) is crossed with a Brassica plant lacking the mutant GTR gene (acceptor plant line). The following introgression scheme is used (the mutant GTR gene is abbreviated to gtr while the wild type is depicted as GTR):

Initial cross: gtr/gtr (donor plant) X GTR/GTR (acceptor plant, e.g. breeding line)

F1 plant: GTR/gtr

BC1 cross: GTR/gtr X GTR/GTR (acceptor plant)

BC1 plants: 50% GTR/gtr and 50% GTR/GTR

The 50% GTR/gtr are selected using molecular markers (e.g. AFLP, PCR, Invader™ and the like) for the mutant GTR allele (gtr).

BC2 cross: GTR/gtr (BC1 plant) X GTR/GTR (acceptor plant)

BC2 plants: 50% GTR/gtr and 50% GTR/GTR

The 50% GTR/gtr are selected using molecular markers for the mutant GTR allele (gtr).

Backcrossing is repeated until BC3 to BC6

BC3-6 plants: 50% GTR/gtr and 50% GTR/GTR

The 50% GTR/gtr are selected using molecular markers for the mutant GTR allele (gtr). To reduce the number of backcrossings (e.g. until BC3 in stead of BC6), molecular markers can be used specific for the genetic background of the acceptor plant line.

BC3-6 S1 cross: GTR/gtr X GTR/gtr

BC3-6 S1 plants: 25% GTR/GTR and 50% GTR/gtr and 25% gtr/gtr

Plants containing gtr are selected using molecular markers for the mutant GTR allele (gtr). Individual BC3-6 S1 plants that are homozygous for the mutant GTR allele (gtr/gtr) are selected using molecular markers for the mutant and the wild-type GTR alleles. These plants are then used for seed production.

Characterization of GSL Content in Parts of Mutant GTR Brassica Plants

The GSL content of seed, stems, flowers, leaves, roots, siliques and silique walls is analysed in mutant GTR Brassica plants as described above for the mutant GTR Arabidopsis plants. Brassica plants comprising specific combinations of mutant GTR genes resulting in specific GSL contents in specific plant parts are selected for further breeding, for seed production or for crop cultivation.

WT and mutated (by Targeting Induced Local Lesions IN Genomes, TILLING) Brassica rapa seeds of the ecotype “R-0-18” (inbred line of the Brassica rapa subsp. trilocularis) were purchased from Reverse Genetics UK (RevGenUK). Seeds were sown on plant substrate and watered with Bactimos (B. thuringiensis israelensis; after sowing, the seeds were cold-stratified at 4° C. for two days to obtain a high and uniform germination rate. Finally, germinating seeds were transferred to a growth chamber with 16 hours day length and a temperature of 20° C. B. rapa plants used for analyses were 2.5 months old; they had green siliques containing immature green seeds. The number of replicate plants used in the experiment was n=5 plants.

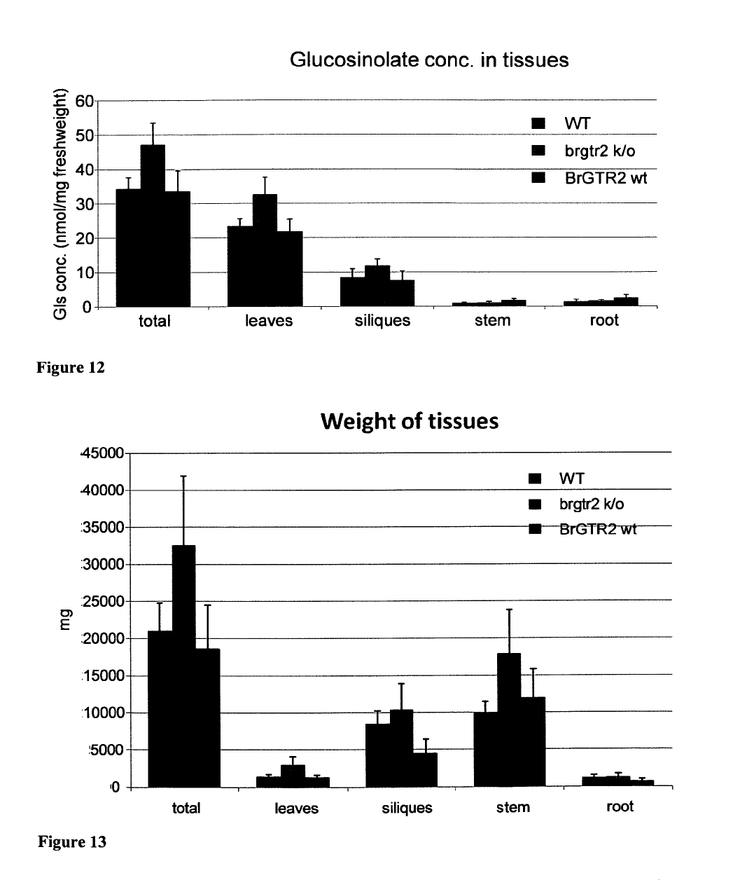

B. rapa leaves and other tissues were weighted and homogenized in liquid N2 using either a mortar (leaves and roots) or a blender (stem and siliques). Glucosinolates from the ice-cold homogenized plant material were extracted in 85% methanol containing p-hydroxybenzyl glucosinolate (pOHB) as internal standard. Part of the supernatant was transferred to a 96-well filter plate loaded with 45 μl DEAE Sephadex™ A-25 column material. Glucosinolates were bound to the column material while samples were sucked through the filter plate by applying brief vacuum. Afterwards, columns were washed with 70% methanol and water. A sulfatase solution (2 mg/ml) was added to the columns and allowed to incubate at room temperature overnight. 100 μl water were applied to the columns and a short spin eluted the desulfo-glucosinolates into a 96-well format plate. The samples were analyzed on an Agilent technologies 1200 series HPLC-DAD system and separated on a Zorbax SB-AQ column. FIG. 12 summarizes the glucosinolate concentration in the different tissues of wt and knock-out B. rapa, while FIG. 13 summarizes the weight of these tissues

### Example 5

### Generation and Characterization of Mutant GTR1 and GTR2 Alleles with Altered Phosphorylation/Dephosphorylation Status

Arabidopsis thaliana GTR1 and GTR2 alleles which mimic constitutive phosphorylation or dephosphorylation were constructed based on experimentally verified phosphorylation sites fsearchable in the http://phosphat.mpimp-golm.mpg.de/—a database collecting phosphoproteomics data. Phosphorylation was mimicked by substituting the possible phosphorylation site amino acid (S or T) with aspartic acid. Dephosphorylation was mimicked by substituting for alanine. The following constructs were made:

Constructs encoding a GTR1 protein with the amino acid sequence of SEQ ID NO: 2 with the following substitutions:
a. S at position 22 for A
b. S at position 22 for D
c. T at position 105 for A
d. T at position 105 for D
e. S at position 605 for A
f. S at position 605 for D
   (which corresponds to the amino acid sequence of SEQ ID No: 142 with the following substitution:
g. S at position 52 for A
h. S at position 52 for D
i. T at position 135 for A
j. T at position 135 for D
k. S at position 635 for A
l. S at position 635 for D
   or constructs encoding a GTR2 protein with the amino acid sequence of SEQ ID No: 4 with the following substitutions:
m. T at position 58 for A
n. T at position 58 for D
o. T at position 117 for A
p. T at position 117 for D
q. T at position 323 for A
r. T at position 323 for D
   Also tested were GTR1 or GTR2 encoding constructs wherein the Proline at position 492 (or 522) was substituted by Leucine.

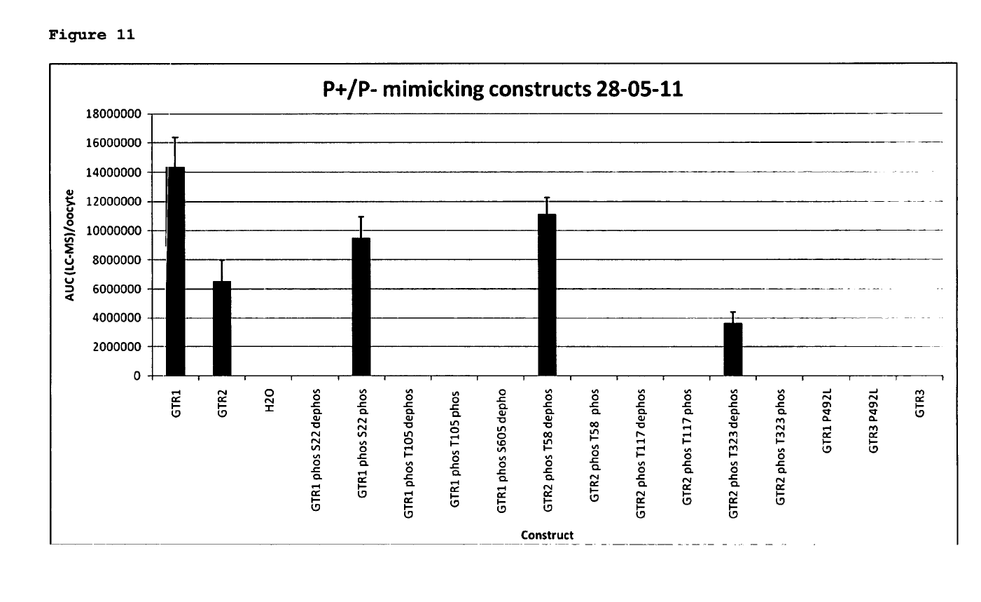

Phosphorylation/dephosphorylation constructs were assayed for proton dependent import of the glucosinolate 4-MTB (4-methylsulfinylbutyl glucosinolate) at a concentration of 100 μM. 5 Oocytes were injected with cRNA (times 4 replications) of one of the constructs (at a 100 ng/μl) and kept at 17 degrees for 5 days. On day 5 the oocytes were preincubated for 5 minutes in Kulori pH 5 and then transferred to the assay media (Kulori pH5 with 4-MTB at a concentration of 100 μM). After one hour oocytes were removed from the media containing 4-MTB and washed 4 times before being extracted in 100% methanol. LC-MS analysis to detect the uptake of 4-MTB was carried out and the results presented in FIG. 11.

It can be observed that constitutive dephosphorylation of GTR1 at position S22 (S52*) turns of f transport completely. Likewise it can be observed that phosphorylation of the same residue keeps the import intact. Furthermore, it can be observed that dephosphorylation of GTR2T58 keeps the transport activity intact whereas phosphorylation of the same residue inactivates the transport. Both constitutive phosphorylation and dephosphorylation of T105 (T135*) in GTR1 and T117 in GTR2 block the transport activity. It could also be observed that mutating GTR1P492 (P522*) and GTR3P492 to a leucine inactivates transport activity. In summary it can be concluded that phosphorylation/dephosphorylation at specific residues regulates GTR1 and GTR2 mediated glucosinolate transport. (amino acid position indicated by * are with reference to the long version of GTR1 including the N-terminal extension of 30 AA)

### Example 6

### Downregulation of GTR1 and/or GTR2 Expression in Brassica spp. Using dsRNA Constructs

Using standard recombinant DNA techniques dsRNA encoding recombinant genes are constructed comprising the following operably linked DNA fragments

a) GTR1 downregulating recombinant gene
i. a plant expressible promoter such as CaMV35S
ii. a DNA region comprising the nucleotide sequence of SEQ ID 138
iii. a transcription termination and polyadenylation region, such as the 3′ nos region.

b) GTR2 downregulating recombinant gene
i. a plant expressible promoter such as CaMV35S
ii. a DNA region comprising the nucleotide sequence of SEQ ID 139
iii. a transcription termination and polyadenylation region, such as the 3′ nos region.

c) GTR1/GTR2 downregulating recombinant gene
i. a plant expressible promoter such as CaMV35S
ii. a DNA region comprising the nucleotide sequence of SEQ ID 140
iii. a transcription termination and polyadenylation region, such as the 3′ nos region.

These dsRNA encoding recombinant genes are introduced (separately) between the borders of a T-DNA vector together with a selectable marker gene such as the phosphinotricin acetyltransferase encoding selectable marker gene. The T-DNA vector is introduced into an Agrobacterium strain comprising a helper Ti-plasmid using conventional methods. Hypocotyl explants of Brassica napus are obtained, cultured and transformed essentially as described by De Block et al. (1989), Plant Physiol. 91: 694) to transfer the chimeric genes into Brassica napus plants.

Transgenic Brassica napus plant are identified and analyzed for glucosinolate content in leaves and seeds.

The invention thus includes the embodiments as described in the following paragraphs:
1. A method for modifying the glucosinolate (GSL) content in a Brassicales plant or part thereof comprising modifying the functional activity of at least one glucosinolate transport (GTR) protein comprising an amino acid sequence having at least 33% sequence identity with SEQ ID NO: 2 or SEQ ID NO: 142 in cells of said plant or said plant part.
2. The method of paragraph 1, wherein the GTR protein comprises an amino acid sequence having at least 80% sequence identity with SEQ ID NO: 2, 4, 6, 8, 10, 12 or 142.
3. The method of paragraph 1 or 2, wherein the GTR protein comprises an amino acid sequence having at least 80% sequence identity with any one of SEQ ID No. 14, 16, 18, 20, 22, 24, 26, 28, 30, 32, 34, 36, 38, 40, 42, 44, 46, 48, 50, 52, 54, 56, 58, 60, 120, 122, 124, 126, 128, 130, 144, 146, 148 or 150.
4. The method of paragraph 1 or 2, wherein the GTR protein comprises an amino acid selected from SEQ ID No. 14, 16, 18, 20, 22, 24, 26, 28, 30, 32, 34, 36, 38, 40, 42, 44, 46, 48, 50, 52, 54, 56, 58, 60, 120, 122, 124, 126, 128, 130, 144, 146, 148 or 150.
5. The method of any one of paragraphs 1 to 4, wherein the GTR protein is encoded by a nucleic acid having at least 80% sequence identity with any of SEQ ID No. 13, 15, 17, 19, 21, 23, 25, 27, 29, 31, 33, 35, 37, 39, 41, 43, 45, 47, 49, 51, 53, 55, 57, 59, 119, 121, 123, 125, 127, 129, 131, 132, 133, 134, 135, 136, 137, 143, 145, 147 or 149.
6. The method of any one of paragraphs 1 to 5, wherein the GTR protein is encoded by a nucleic acid selected from SEQ ID No. 13, 15, 17, 19, 21, 23, 25, 27, 29, 31, 33, 35, 37, 39, 41, 43, 45, 47, 49, 51, 53, 55, 57, 59, 119, 121, 123, 125, 127, 129, 131, 132, 133, 134, 135, 136, 137, 143, 145, 147 or 149.
7. The method of any one of paragraphs 1 to 6, comprising modifying the functional activity of at least two GTR proteins.
8. The method of paragraph 7, wherein a first GTR protein comprises an amino acid sequence having at least 80% sequence identity with SEQ ID NO: 2 or SEQ ID NO: 142 and a second GTR protein comprises an amino acid sequence having at least 80% sequence identity with SEQ ID NO: 4.
9. The method of paragraph 7, wherein said first GTR protein comprises an amino acid sequence having at least 80% sequence identity with any one of SEQ ID No. 14, 16, 18, 20, 22, 24, 38, 40, 42, 50, 52, 54 or 144, 146, 148 or 150 and wherein said second GTR protein comprises an amino acid sequence having at least 80% sequence identity with any one of SEQ ID No. 26, 28, 30, 32, 34, 36, 44, 46, 48, 56, 58, 60, 120, 122, 124, 126, 128, or 130.
10. The method of paragraph 7, wherein said first GTR protein comprises an amino acid sequence selected from SEQ ID No. 14, 16, 18, 20, 22, 24, 38, 40, 42, 50, 52 or 54 or 144, 146, 148, 150 and wherein said second GTR protein comprises an amino acid sequence selected from SEQ ID No. 26, 28, 30, 32, 34, 36, 44, 46, 48, 56, 58, 60, 120, 122, 124, 126, 128, or 130.
11. The method of paragraph 7, wherein said first GTR protein is encoded by a nucleic acid having at least 80% sequence identity with any one of SEQ ID No. 13, 15, 17, 19, 21, 23, 37, 39, 41, 49, 51, 53 or 137 or 143, 145, 147, 149 and wherein said second GTR protein is encoded by a nucleotide sequence having at least 80% sequence identity with any one of SEQ ID No. 25, 27, 29, 31, 33, 35, 43, 45, 47, 55, 57, 59, 119, 121, 123, 125, 127, 129, 131, 132, 133, 134, 135, 136.
12. The method of paragraph 7, wherein said first GTR protein is encoded by a nucleic acid selected from SEQ ID No. 13, 15, 17, 19, 21, 23, 37, 39, 41, 49, 51, 53 or 137 or 143, 145, 147, 149 and wherein said second GTR protein is encoded by a nucleotide sequence selected from SEQ ID No. 25, 27, 29, 31, 33, 35, 43, 45, 47, 55, 57, 59, 119, 121, 123, 125, 127, 129, 131, 132, 133, 134, 135, 136.
13. The method of any one of paragraphs 1 to 12, comprising reducing the functional GTR activity in cells of said plant or plant part.
14. The method of paragraph 13, wherein the GSL content is decreased in plant seed.
15. The method of paragraph 13, wherein the GSL content is increased in green plant tissue.
16. The method of any one of paragraphs 9 to 12, wherein said reduction of functional GTR activity comprises down-regulation of GTR gene expression.
17. The method of any one of paragraphs 1 to 16, wherein said reduction of functional GTR activity comprises down-regulation of GTR protein activity.
18. The method according to paragraph 17, comprising introducing an RNA molecule in said plant or plant part, wherein said RNA molecule comprises a GTR-inhibitory RNA molecule capable of down-regulating the expression of said GTR gene.
19. The method of paragraph 18, comprising introducing a chimeric DNA construct in said plant or plant part, wherein said chimeric DNA construct comprises the following operably linked DNA regions:
   a) a promoter, operative in said plant or plant part;
   b) a transcribed DNA region, which when transcribed yields a GTR-inhibitory RNA molecule, said GTR-inhibitory RNA molecule being capable of down-regulating the expression of said GTR gene;
   c) a DNA region involved in transcription termination and polyadenylation.
20. The method of paragraph 19, wherein said transcribed DNA region encodes a sense RNA molecule, said DNA region comprising a nucleotide sequence of at least 20 nucleotides with at least 95% identity to the DNA strand of said GTR gene.
21. The method of paragraph 19, wherein said transcribed DNA region encodes an antisense RNA molecule, said DNA region comprising a nucleotide sequence of at least 20 nucleotides with at least 95% identity to the complement of the DNA strand of said GTR gene.
22. The method of paragraph 19, wherein said transcribed DNA region encodes a double-stranded RNA molecule, comprising:
   a) a sense RNA region comprising at least 20 consecutive nucleotides having at least 95% identity to said GTR gene;
   b) an antisense RNA region comprising at least 20 nucleotides complementary to said sense RNA region;
      wherein said sense and antisense RNA regions are capable of forming a double stranded RNA region and wherein said double-stranded RNA molecule is capable of down-regulating the expression of said GTR gene.
23. The method of paragraph 22 wherein said transcribed DNA region comprises a nucleotide sequence selected from the nucleotide sequence of SEQ ID No. 138, 139 or 140.
24. The method of paragraph 19, wherein said transcribed DNA region encodes a pre-miRNA molecule which is processed into a miRNA capable of guiding the cleavage of mRNA transcribed from said GTR gene.
25. The method of any one of paragraphs 19 to 24, wherein said promoter is a tissue-specific or inducible promoter.
26. The method of paragraph 25, wherein said promoter is a seed-specific promoter.
27. The method of paragraphs 16 or 17, comprising altering the nucleotide sequence of the endogenous GTR gene.
28. The method according to paragraph 27 wherein the GTR protein is a protein comprising the amino acid sequence of SEQ ID NO: 2 with any of the following substitutions:
   a) S at position 22 for A
   b) S at position 22 for D
   c) T at position 105 for A
   d) T at position 105 for D
   e) S at position 605 for A
   f) S at position 605 for D
   or wherein the GTR protein is a protein comprising the amino acid sequence of SEQ ID NO: 142 with any of the following substitutions:
   g) S at position 52 for A
   h) S at position 52 for D
   i) T at position 135 for A
   j) T at position 135 for D
   k) S at position 635 for A
   l) S at position 635 for D.
29. The method according to paragraph 27 wherein the GTR protein is a protein with the amino acid sequence of SEQ ID No: 4 with the following substitutions:
   a) T at position 58 for A
   b) T at position 58 for D
   c) T at position 117 for A
   d) T at position 117 for D
   e) T at position 323 for A
   f) T at position 323 for D
30. The method according to paragraph 27 wherein the GTR protein is encoded by a nucleic acid of SEQ ID No. 25
   a) comprising a stop codon at position 1241 to 1243
31. The method according to paragraph 27 wherein the GTR protein is encoded by a nucleic acid of SEQ ID No. 31
   a) comprising a stop codon at position 929 to 931
   b) comprising a stop codon at position 1145 to 1147
32. The method according to paragraph 27 wherein the GTR protein is encoded by a nucleic acid of SEQ ID No. 27
   a) comprising a stop codon at position 870 to 872
   b) comprising a stop codon at position 1380 to 1382
33. The method according to paragraph 27 wherein the GTR protein is encoded by a nucleic acid of SEQ ID No. 33
   a) comprising a stop codon at position 780 to 782
34. The method according to paragraph 27 wherein the GTR protein is a protein with the amino acid sequence of SEQ ID No. 66 comprising a mutation at any of the following positions:
   a) Gly 126
   b) Gly 145
   c) Glu 192
   d) Trp 229
   e) Ser 359
35. The method of any one of paragraphs 1 to 34, wherein said plant is a Brassica plant.
36. A plant or plant part obtainable by the method of any one of paragraphs 1 to 34.
37. Seed from the plant of paragraph 36.
38. Seed meal from the seed of paragraph 37.
39. Green plant tissue from the plant of paragraph 36.
40. Oil from the plant of paragraph 35 or the seed of paragraph 37.
41. Use of the plant or plant part of paragraph 36 in animal feed.
42. Use of the plant or plant part of paragraph 36 in pest management, in particular in biofumigation.
43. Use of the plant or plant part of paragraph 36 in cancer-prevention.
44. Use of a GTR-encoding nucleic acid sequence to obtain a modified GSL content in a plant or plant part.
45. A method for producing seed or seed meal comprising growing a plant with reduced functional GTR activity and recovering seed from said plant.
46. A chimeric DNA construct as described in any one of paragraphs 19 to 32.
47. A nucleic acid encoding a mutant GTR protein, wherein the corresponding wildtype GTR protein comprises an amino acid sequence having at least 33% sequence identity with SEQ ID NO: 2 or SEQ ID NO:142.
48. The nucleic acid of paragraph 33, wherein the wildtype GTR protein comprises an amino acid sequence having at least 80% sequence identity with SEQ ID NO: 2, 4, 6, 8, 10 or 12 or SEQ ID NO: 142.
49. A mutant GTR protein encoded by the nucleic acid of paragraph 47 or 48.
50. A method for identifying a nucleic acid according to paragraph 47 or 48 in a biological sample comprising determining the presence of a mutated DNA region in a nucleic acid of the sample, said mutated DNA region comprising at least one deleted, inserted or substituted nucleotide in the mutant GTR nucleic acid compared to a nucleic acid encoding the corresponding wildtype GTR protein.
51. A kit for identifying a nucleic acid according to paragraph 45 or 46 in a biological sample, comprising a set of primers or probes, said set selected from the group consisting of:
   a set of primers or probes, wherein one of said primers or probes specifically recognizes a DNA region 5′ flanking the mutated DNA region and the other of said primers or probes specifically recognizes a DNA region 3′ flanking the mutated DNA region,
   a set of primers or probes, wherein one of said primers or probes specifically recognizes a DNA region 5′ or 3′ flanking the mutated DNA region and the other of said primers or probes specifically recognizes the mutated DNA region,
   a set of primers or probes, wherein one of said primers or probes specifically recognizes a DNA region 5′ or 3′ flanking the mutated DNA region and the other of said primers or probes specifically recognizes the joining region between the 3′ or 5′ flanking region and the mutated DNA region, respectively,
   a probe which specifically recognizes the joining region between a DNA region 5′ or 3′ flanking the mutated DNA region and the mutated DNA region.
52. An isolated DNA sequence encoding the amino acid sequence of SEQ ID No. 2, 4, 6, 8, or 12 or 142.
53. An isolated DNA sequence encoding the amino acid sequence of SEQ ID No. 14, 16, 18, 20, 22, 24, 26, 28, 30, 32, 34, 36, 38, 40, 42, 44, 46, 48, 50, 52, 54, 56, 58, 60, 120, 122, 124, 126, 128, 130 or 144, 146, 148 or 150.
54. An isolated DNA sequence comprising the nucleotide sequence of SEQ ID No. 25, 27, 29, 31, 33, 35, 43, 45, 47, 55, 57, 59, 119, 121, 123, 125, 127, 129, 131, 132, 133, 134, 135, 136 or 143, 145, 147 or 149.
55. A chimeric gene comprising the following operably linked DNA fragments:
   a) a heterologous plant expressible promoter.
   b) a DNA region encoding the amino acid sequence of SEQ ID No. 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 120, 122, 124, 126, 128, 130 or 142 or 144, 146, 148 or 150.
   c) a transcription termination and polyadenylation signal functional in plant cells.
56. A plant comprising the chimeric gene of paragraph 55.
57. An isolated protein comprising the amino acid sequence of SEQ ID No. 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 120, 122, 124, 126, 128, 130 or 142 or 144, 146, 148 or 150.
58. A mutant GTR allele encoding a GTR protein with an amino acid sequence having at least 80% sequence identity with SEQ ID NO: 2 with any of the following substitutions:
   a) S at position 22 for A
   b) S at position 22 for D
   c) T at position 105 for A
   d) T at position 105 for D
   e) S at position 605 for A
   f) S at position 605 for D
      or encoding a GTR protein with an amino acid sequence having at least 80% sequence identity with SEQ ID NO: 142 with any of the following substitutions:
   g) S at position 52 for A
   h) S at position 52 for D
   i) T at position 135 for A
   j) T at position 135 for D
   k) S at position 635 for A
   l) S at position 635 for D
59. A mutant GTR allele encoding a GTR protein having at least 80% sequence identity with the amino acid sequence of SEQ ID No: 4 with the following substitutions:
   a) T at position 58 for A
   b) T at position 58 for D
   c) T at position 117 for A
   d) T at position 117 for D
   e) T at position 323 for A
   f) T at position 323 for D
60. A mutant GTR allele having at least 80% sequence identity with the nucleic acid of SEQ ID No. 25
   a) comprising a stop codon at position 1241 to 1243
61. A mutant GTR allele having at least 80% sequence identity with the nucleic acid of SEQ ID No. 31
   a) comprising a stop codon at position 929 to 931
   b) comprising a stop codon at position 1145 to 1147
62. A mutant GTR allele having at least 80% sequence identity with the nucleic acid of SEQ ID No. 27
   a) comprising a stop codon at position 870 to 872
   b) comprising a stop codon at position 1380 to 1382
63. A mutant GTR allele having at least 80% sequence identity with the nucleic acid of SEQ No. 33
   a) comprising a stop codon at position 780 to 782
64. A mutant GTR allele encoding a GTR protein having at least 80% sequence identity with the amino acid sequence of SEQ ID No. 66 comprising a mutation at any of the following positions:
   a) Gly 126
   b) Gly 145
   c) Glu 192
   d) Trp 229
   e) Ser 359

1. A method for modifying the glucosinolate (GSL) content in a Brassicales plant, such as a Brassica plant, or part thereof comprising modifying the functional activity of at least one glucosinolate transport (GTR) protein comprising an amino acid sequence having at least 33% sequence identity with SEQ ID NO: 2 or SEQ ID NO: 142 in cells of said plant or said plant part.

2. The method of claim 1, wherein the GTR protein comprises an amino acid sequence having at least 80% sequence identity with SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 120, 122, 124, 126, 128 or 130 or 142, 144, 146, 148 or 150.

3. (canceled)

4. (canceled)

5. The method of claim 2, wherein the GTR protein is encoded by a nucleic acid having at least 80% sequence identity with any one of SEQ ID NO: 13, 15, 17, 19, 21, 23, 25, 27, 29, 31, 33, 35, 37, 39, 41, 43, 45, 47, 49, 51, 53, 55, 57, 59, 119, 121, 123, 125, 127, 129, 131, 132, 133, 134, 135, 136 or 137 or 143, 145, 147 or 149.

6. (canceled)

7. The method of claim 1, comprising modifying the functional activity of at least two GTR proteins.

8. The method of claim 7, wherein a first GTR protein comprises an amino acid sequence having at least 80% sequence identity with SEQ ID NO: 2 or SEQ ID NO: 142 and a second GTR protein comprises an amino acid sequence having at least 80% sequence identity with SEQ ID NO: 4.

9. The method of claim 7, wherein said first GTR protein comprises an amino acid sequence having at least 80% sequence identity with any one of SEQ ID NO: 14, 16, 18, 20, 22, 24, 38, 40, 42, 50, 52 or 54 or 144, 146, 148 or 150 and wherein said second GTR protein comprises an amino acid sequence having at least 80% sequence identity with any one of SEQ ID NO: 26, 28, 30, 32, 34, 36, 44, 46, 48, 56, 58, 60, 120, 122, 124, 126, 128, or 130.

10. (canceled)

11. The method of claim 7, wherein said first GTR protein is encoded by a nucleic acid having at least 80% sequence identity with any one of SEQ ID NO: 13, 15, 17, 19, 21, 23, 37, 39, 41, 49, 51, 53 or 137 or 143, 145, 147 or 149 and wherein said second GTR protein is encoded by a nucleotide sequence having at least 80% sequence identity with any of SEQ ID NO: 25, 27, 29, 31, 33, 35, 43, 45, 47, 55, 57, 59, 119, 121, 123, 125, 127, 129, 131, 132, 133, 134, 135, 136.

12. (canceled)

13. The method of claim 1, wherein the GSL content is decreased in plant seed.

14. A method to reduce GTR activity in a cell of a plant, such as a Brassica plant, comprising introducing an RNA molecule in said plant or plant part, wherein said RNA molecule comprises a GTR-inhibitory RNA molecule capable of down-regulating the expression of said GTR gene.

15. The method of claim 14, comprising introducing a chimeric DNA construct in said plant or plant part, wherein said chimeric DNA construct comprises the following operably linked DNA regions:
a) a promoter, operative in said plant or plant part;
b) a transcribed DNA region, which when transcribed yields a GTR-inhibitory RNA molecule, said GTR-inhibitory RNA molecule being capable of down-regulating the expression of said GTR gene;
c) a DNA region involved in transcription termination and polyadenylation.

16. A method to reduce GTR activity in a cell of a plant, such as a Brassica plant, comprising altering the nucleotide sequence of the endogenous GTR gene.

17. The method according to claim 16 wherein the GTR protein is a protein comprising the amino acid sequence of SEQ ID NO: 2 or SEQ ID NO: 142 with any of the following substitutions:
a. S at position 22 for A (position 52 for SEQ ID NO:142)
b. S at position 22 for D (position 52 for SEQ ID NO:142)
c. T at position 105 for A (position 135 for SEQ ID NO:142)
d. T at position 105 for D (position 135 for SEQ ID NO:142)
e. S at position 605 for A (position 635 for SEQ ID NO:142)
f. S at position 605 for D (position 635 for SEQ ID NO:142)
or the GTR protein is a protein with the amino acid sequence of SEQ ID NO: 4 with the following substitutions:
g) T at position 58 for A
h) T at position 58 for D
i) T at position 117 for A
j) T at position 117 for D
k) T at position 323 for A
l) T at position 323 for D
or the GTR protein is encoded by a nucleic acid of SEQ ID NO:25
m) comprising a stop codon at position 1241 to 1243
or GTR protein is encoded by a nucleic acid of SEQ ID NO:31
n) comprising a stop codon at position 929 to 931
o) comprising a stop codon at position 1145 to 1147.
or GTR protein is encoded by a nucleic acid of SEQ ID NO:27
p) comprising a stop codon at position 870 to 872
q) comprising a stop codon at position 1380 to 1382
or the GTR protein is encoded by a nucleic acid of SEQ ID NO:33
r) comprising a stop codon at position 780 to 782.
or the GTR protein is a protein with the amino acid sequence of SEQ ID NO:66 comprising a mutation at any of the following positions:
s) Gly 126
t) Gly 145
u) Glu 192
v) Trp 229
w) Ser 359

18. (canceled)

19. A plant or plant part obtainable by the method of claim 1.

20. Seed from the plant of claim 19.

21. (canceled)

22. An isolated DNA sequence encoding the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 120, 122, 124, 126, 128 or 130, or −142, 144, 146, 148 or 150.

23. (canceled)

24. An isolated DNA sequence comprising the nucleotide sequence of SEQ ID NO: 25, 27, 29, 31, 33, 35, 43, 45, 47, 55, 57, 59, 119, 121, 123, 125, 127, 129, 131, 132, 133, 134, 135, 136 or 143, 145, 147 or 149.

25. A chimeric gene comprising the following operably linked DNA fragments:
a) a heterologous plant expressible promoter;
b) a DNA region encoding the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 120, 122, 124, 126, 128, 130 or 142 or 144, 146, 148 or 150;
c) a transcription termination and polyadenylation signal functional in plant cells.

26. A plant comprising the chimeric gene of claim 25.

27. An isolated protein comprising the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 120, 122, 124, 126, 128, 130 or 142 or 144, 146, 148 or 150.

28. A mutant GTR allele encoding a GTR protein comprising an amino acid sequence having at least 80% sequence identity with SEQ ID NO: 2 or SEQ ID NO: 142 with any one of the following substitutions:
a) S at position 22 for A (position 52 for SEQ ID NO:142)
b) S at position 22 for D (position 52 for SEQ ID NO:142)
c) T at position 105 for A (position 135 for SEQ ID NO:142)
d) T at position 105 for D (position 135 for SEQ ID NO:142)
e) S at position 605 for A (position 635 for SEQ ID NO:142)
f) S at position 605 for D (position 635 for SEQ ID NO:142)
or a GTR protein having at least 80% sequence identity with the amino acid sequence of SEQ ID NO: 4 with the following substitutions:
g) T at position 58 for A
h) T at position 58 for D
i) T at position 117 for A
j) T at position 117 for D
k) T at position 323 for A
l) T at position 323 for D
or a mutant GTR allele having at least 80% sequence identity with the nucleic acid of SEQ ID NO: 25 comprising a stop codon at position 1241 to 1243;
or mutant GTR allele having at least 80% sequence identity with the nucleic acid of SEQ ID NO: 31
m) comprising a stop codon at position 929 to 931
n) comprising a stop codon at position 1145 to 1147
or a mutant GTR allele having at least 80% sequence identity with the nucleic acid of SEQ ID NO: 27
o) comprising a stop codon at position 870 to 872
p) comprising a stop codon at position 1380 to 1382
or a mutant GTR allele having at least 80% sequence identity with the nucleic acid of SEQ NO: 33
q) comprising a stop codon at position 780 to 782.
Or a mutant GTR allele encoding a GTR protein having at least 80% sequence identity with the amino acid sequence of SEQ ID NO: 66 comprising a mutation at any of the following positions:
r) Gly 126
s) Gly 145Glu 192
t) Trp 229
u) Ser 359

29. A plant or plant part obtainable by the method of claim 14.

30. Seed from the plant of claim 29.

31. A plant or plant part obtainable by the method of claim 16.

32. Seed from the plant of claim 31.
